SEQUENCE LISTING (132 sequences)

SEQ ID NO: 1 - protein, 45 aa, synthetic construct
  source 1..45: note = CD8
TTTPAPRPPTPAPTIASQPLSLRPEACRPAAGGAVHTRGLDFACD

SEQ ID NO: 2 - protein, 21 aa, synthetic construct
  source 1..21: note = signal
MALPVTALLLPLALLLHAARP

SEQ ID NO: 3 - DNA, 63 bp, synthetic construct
  source 1..63: mol_type = other DNA; note = signal
atggcactgccagtgaccgccctgctgctgcctctggccctgctgctgcacgcagcaaggcca

SEQ ID NO: 4 - protein, 66 aa, synthetic construct
  source 1..66: note = CD8L
ALSNSIMYFSHFVPVFLPAKPTTTPAPRPPTPAPTIASQPLSLRPEACRPAAGGAVHTRGLDFACD

SEQ ID NO: 5 - protein, 12 aa, synthetic construct
  source 1..12: note = CD8S
ESKYGPPCPPCP

SEQ ID NO: 6 - protein, 30 aa, synthetic construct
  source 1..30: note = CD28
LDNEKSNGTIIHVKGKHLCPSPLFPGPSKP

SEQ ID NO: 7 - protein, 40 aa, synthetic construct
  source 1..40: note = CD28
KIEVMYPPPYLDNEKSNGTIIHVKGKHLCPSPLFPGPSKP

SEQ ID NO: 8 - protein, 39 aa, synthetic construct
  source 1..39: note = CD28
IEVMYPPPYLDNEKSNGTIIHVKGKHLCPSPLFPGPSKP

SEQ ID NO: 9 - protein, 92 aa, synthetic construct
  source 1..92: note = FcgammaR1
APVLNASVTSPLLEGNLVTLSCETKLLLQRPGLQLYFSFYMGSKTLRGRNTSSEYQILTARREDSGLYWCEAATEDGNVLKRSPELELQVLG

SEQ ID NO: 10 - protein, 117 aa, synthetic construct
  source 1..117: note = CSF1R
PPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTP

SEQ ID NO: 11 - protein, 21 aa, synthetic construct
  source 1..21: note = CD8
IYIWAPLAGTCGVLLLSLVIT

SEQ ID NO: 12 - protein, 21 aa, synthetic construct
  source 1..21: note = SLAMF7
VLLCLLLVPLLLSLFVLGLFL

SEQ ID NO: 13 - protein, 21 aa, synthetic construct
  source 1..21: note = CD86
WITAVLPTVIICVMVFCLILW

SEQ ID NO: 14 - protein, 21 aa, synthetic construct
  source 1..21: note = DECTIN-1 /CLEC7A
GLVVAIVLIVLCLIGLIVAIL

SEQ ID NO: 15 - protein, 21 aa, synthetic construct
  source 1..21: note = DECTIN-2 /CLEC6A
VVCSVIFCASLLAISIGAVSW

SEQ ID NO: 16 - protein, 21 aa, synthetic construct
  source 1..21: note = DECTIN-3/ CLEC4D
SAIFCVSLLLIFVVAIVSPIL

SEQ ID NO: 17 - protein, 22 aa, synthetic construct
  source 1..22: note = CD40
ALVVIPIIFGILFAILLVLVFI

SEQ ID NO: 18 - protein, 20 aa, synthetic construct
  source 1..20: note = CD40
AVILFFFTFFITTMVMLAAL

SEQ ID NO: 19 - protein, 21 aa, synthetic construct
  source 1..21: note = TLR1
LLIVTIVATMLVLAVTVTSLC

SEQ ID NO: 20 - protein, 20 aa, synthetic construct
  source 1..20: note = TLR2
ALVSGMCCALFLLILLTGVL

SEQ ID NO: 21 - protein, 20 aa, synthetic construct
  source 1..20: note = TLR3
FFMINTSILLIFIFIVLLIH

SEQ ID NO: 22 - protein, 21 aa, synthetic construct
  source 1..21: note = TLR4
TIIGVSVLSVLVVSVVAVLVY

SEQ ID NO: 23 - protein, 21 aa, synthetic construct
  source 1..21: note = TLR5
FSLFIVCTVTLTLFLMTILTV

SEQ ID NO: 24 - protein, 21 aa, synthetic construct
  source 1..21: note = TLR6
LIVTIGATMLVLAVTVTSLCI

SEQ ID NO: 25 - protein, 21 aa, synthetic construct
  source 1..21: note = TLR7
LILFSLSISVSLFLMVMMTAS

SEQ ID NO: 26 - protein, 27 aa, synthetic construct
  source 1..27: note = CD28
FWVLVVVGGVLACYSLLVTVAFIIFWV

SEQ ID NO: 27 - protein, 41 aa, synthetic construct
  source 1..41: note = CD28
RSKRSRLLHSDYMNMTPRRPGPTRKHYQPYAPPRDFAAYRS

SEQ ID NO: 28 - protein, 23 aa, synthetic construct
  source 1..23: note = MDL1
FYLLFLTMGVVKLVVVILGSIIM

SEQ ID NO: 29 - protein, 21 aa, synthetic construct
  source 1..21: note = DAP12
GVLAGIVMGDLVLTVLIALAV

SEQ ID NO: 30 - protein, 20 aa, synthetic construct
  source 1..20: note = IL1R1
HMIGICVTLTVIIVCSVFIY

SEQ ID NO: 31 - protein, 23 aa, synthetic construct
  source 1..23: note = BDCA2
HPVVSSVTFCVSLLLISVVAMSW

SEQ ID NO: 32 - protein, 21 aa, synthetic construct
  source 1..21: note = VEGFR2
IIILVGTAVIAMFFWLLLVII

SEQ ID NO: 33 - protein, 21 aa, synthetic construct
  source 1..21: note = CSF1R
VVVACMSIMALLLLLLLLLLY

SEQ ID NO: 34 - protein, 26 aa, synthetic construct
  source 1..26: note = CSF2R
NLGSVYIYVLLIVGTLVCGIVLGFLF

SEQ ID NO: 35 - protein, 23 aa, synthetic construct
  source 1..23: note = CSF3R
IILGLFGLLLLLTCLCGTAWLCC

SEQ ID NO: 36 - protein, 24 aa, synthetic construct
  source 1..24: note = CD11b
PLPLIVGSSVGGLLLLALITAALY

SEQ ID NO: 37 - protein, 21 aa, synthetic construct
  source 1..21: note = IFNgR1
SLWIPVVAALLLFLVLSLVFI

SEQ ID NO: 38 - protein, 30 aa, synthetic construct
  source 1..30: note = NKG2D
SNLFVASWIAVMIIFRIGMAVAIFCCFFFP

SEQ ID NO: 39 - protein, 21 aa, synthetic construct
  source 1..21: note = MINCLE/CLEC4E
TIFCASLFLIPIGAVTWLFMQ

SEQ ID NO: 40 - protein, 61 aa, synthetic construct
  source 1..61: note = CD86
KWKKKKRPRNSYKCGTNTMEREESEQTKKREKIHIPERSDEAQRVFKSSKTSSCDKSDTCF

SEQ ID NO: 41 - protein, 36 aa, synthetic construct
  source 1..36: note = LOX1
CWWPSYLFQLGKAKKGNSKEDPQDKVTQIKLDDFTM

SEQ ID NO: 42 - protein, 59 aa, synthetic construct
  source 1..59: note = 2B4
WRRKRKEKQSETSPKEFLTIYEDVKDLKTRRNHEQEQTFPGGGSTIYSMIQSQSSAPTS

SEQ ID NO: 43 - protein, 44 aa, synthetic construct
  source 1..44: note = DECTIN-1
RWPPSAACSGKESVVAIRTNSQSDFHLQTYGDEDLNELDPHYEM

SEQ ID NO: 44 - protein, 20 aa, synthetic construct
  source 1..20: note = DECTIN-2
LRLSLWGRKETSQPQQEQMM

SEQ ID NO: 45 - protein, 48 aa, synthetic construct
  source 1..48: note = TIM1
KKYFFKKEVQQLSVSFSSLQIKALQNAVEKEVQAEDNIYIENSLYATD

SEQ ID NO: 46 - protein, 62 aa, synthetic construct
  source 1..62: note = CD40
KKVAKKPTNKAPHPKQEPQEINFPDDLPGSNTAAPVQETLHGCQPVTQEDGKESRISVQERQ

SEQ ID NO: 47 - protein, 24 aa, synthetic construct
  source 1..24: note = CD11b
KLGFFKRQYKDMMSEGGPPGAEPQ

SEQ ID NO: 48 - protein, 434 aa, synthetic construct
  source 1..434: note = CSF1R
KYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 49 - protein, 54 aa, synthetic construct
  source 1..54: note = CSF2R
KRFLRIQRLFPPVPQIKDKLNDNHEVEDEIIWEEFTPEEGKGYREEVLTVKEIT

SEQ ID NO: 50 - protein, 186 aa, synthetic construct
  source 1..186: note = CSF3R
SPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 51 - protein, 213 aa, synthetic construct
  source 1..213: note = IL1R
KIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 52 - protein, 185 aa, synthetic construct
  source 1..185: note = TLR1
SYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 53 - protein, 175 aa, synthetic construct
  source 1..175: note = TLR2
HRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 54 - protein, 176 aa, synthetic construct
  source 1..176: note = TLR2
CHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 55 - protein, 180 aa, synthetic construct
  source 1..180: note = TLR3
FEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 56 - protein, 187 aa, synthetic construct
  source 1..187: note = TLR4
KFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 57 - protein, 198 aa, synthetic construct
  source 1..198: note = TLR5
TKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 58 - protein, 189 aa, synthetic construct
  source 1..189: note = TLR6
YLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 59 - protein, 189 aa, synthetic construct
  source 1..189: note = TLR7
HLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 60 - protein, 194 aa, synthetic construct
  source 1..194: note = TLR8
AHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 61 - protein, 388 aa, synthetic construct
  source 1..388: note = SCARF1
CCCWAPRSDLKDRPARDGATVSRMKLQVWGTLTSLGSTLPCRSLSSHKLPWVTVSHHDPEVPFNHSFIEPPSAGWATDDSFSSDPESGEADEVPAYCVPPQEGMVPVAQAGSSEASLAAGAFPPPEDASTPFAIPRTSSLARAKRPSVSFAEGTKFAPQSRRSSGELSSPLRKPKRLSRGAQSGPEGREAEESTGPEEAEAPESFPAAASPGDSATGHRRPPLGGRTVAEHVEAIEGSVQESSGPVTTIYMLAGKPRGSEGPVRSVFRHFGSFQKGQAEAKVKRAIPKPPRQALNRKKGSPGLASGSVGQSPNSAPKAGLPGATGPMAVRPEEAVRGLGAGTESSRRAQEPVSGCGSPEQDPQKQAEEERQEEPEYENVVPISRPPEP

SEQ ID NO: 62 - protein, 409 aa, synthetic construct
  source 1..409: note = SCARF2
CCCACRGKDPTRRPRPRRELSLGRKKAPHRLCGRFSRISMKLPRIPLRRQKLPKVVVAHHDLDNTLNCSFLEPPSGLEQPSPSWSSRASFSSFDTTDEGPVYCVPHEEAPAESRDPEVPTVPAEAPAPSPVPLTTPASAEEAIPLPASSDSERSASSVEGPGGALYARVARREARPARARGEIGGLSLSPSPERRKPPPPDPATKPKVSWIHGKHSAAAAGRAPSPPPPGSEAAPSPSKRKRTPSDKSAHTVEHGSPRTRDPTPRPPGLPEEATALAAPSPPRARARGRGPGLLEPTDAGGPPRSAPEAASMLAAELRGKTRSLGRAEVALGAQGPREKPAPPQKAKRSVPPASPARAPPATETPGPEKAATDLPAPETPRKKTPIQKPPRKKSREAAGELGRAGAPTL

SEQ ID NO: 63 - protein, 37 aa, synthetic construct
  source 1..37: note = DC-SIGN
CGALSKYGRTQRFGLGRLQEEELLGLQQLRPEKSDSM

SEQ ID NO: 64 - protein, 28 aa, synthetic construct
  source 1..28: note = CXCL16
CKRRRGQSPQSSPDLPVHYIPVAPDSNT

SEQ ID NO: 65 - protein, 51 aa, synthetic construct
  source 1..51: note = CD93
GLLVYRKRRAKREEKKEKKPQNAADSYSWVPERAESRAMENQYSPTPGTDC

SEQ ID NO: 66 - protein, 19 aa, synthetic construct
  source 1..19: note = LIMPII
GQGSMDEGTADERAPLIRT

SEQ ID NO: 67 - protein, 52 aa, synthetic construct
  source 1..52: note = MELLEC
LAVPRWTSSPARHETRRPEPHRTTASGQSHLSMTTDGDDDLMDRTSSYKAQM

SEQ ID NO: 68 - protein, 35 aa, synthetic construct
  source 1..35: note = DNGR1
LCCAGSCKNSSLCKQYTDPAPSDWQLSTYIEEEHM

SEQ ID NO: 69 - protein, 43 aa, synthetic construct
  source 1..43: note = TIM4
LRGKLMETYCSQKHTRLDYIGDSKNVLNDVQHGREDEDGLFTL

SEQ ID NO: 70 - protein, 84 aa, synthetic construct
  source 1..84: note = FCER1G
LRKRRDSLSLSTQRTQGPAESARNLEYVSVSPTNNTVYASVTHSNRETEIWTPRENDTITIYSTINHSKESKPTFSRATALDNV

SEQ ID NO: 71 - protein, 571 aa, synthetic construct
  source 1..571: note = VEGFR2
LRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 72 - protein, 69 aa, synthetic construct
  source 1..69: note = IFNgR2
LVLKYRGLIKYWFHTPPSIPLQIEEYLKDPTQPILEALDKDSSPKDDVWDSVSIISFPEKEQEDVLQTL

SEQ ID NO: 73 - protein, 33 aa, synthetic construct
  source 1..33: note = CLEC2
MVRWWSSSASGVSILAPKRTKINLTIYGDEDQM

SEQ ID NO: 74 - protein, 43 aa, synthetic construct
  source 1..43: note = MARCO
NVGNRRKPKPVNIEFPEMAIQHFAAQQTESLLEDEKLIKKNRM

SEQ ID NO: 75 - protein, 31 aa, synthetic construct
  source 1..31: note = DEC-205
QRHRLHLAGFSSVRYAQGVNEDEIMLPSFHD

SEQ ID NO: 76 - protein, 41 aa, synthetic construct
  source 1..41: note = RAGE
QRRQRRGEERKAPENQEEEEERAELNQSEEPEAGESSTGGP

SEQ ID NO: 77 - protein, 71 aa, synthetic construct
  source 1..71: note = STAB1
RARGKPMGFGFSAFQAEDDADDDFSPWQEGTNPTLVSVPNPVFGSDTFCEPFDDSLLEEDFPDTQRILTVK

SEQ ID NO: 78 - protein, 41 aa, synthetic construct; identical to SEQ ID NO: 27
  source 1..41: note = CD28

SEQ ID NO: 79 - protein, 43 aa, synthetic construct
  source 1..43: note = LANGERIN
RVTPTKGPVLSPGSKPPPERPWLSINQKDVTFHADPAEKEVTM

SEQ ID NO: 80 - protein, 11 aa, synthetic construct
  source 1..11: note = CD36
SYCACRSKTIK

SEQ ID NO: 81 - protein, 26 aa, synthetic construct
  source 1..26: note = CD80
TTYCFAPRCRERRRNERLRRESVRPV

SEQ ID NO: 82 - protein, 25 aa, synthetic construct
  source 1..25: note = CD80
TYCFAPRCRERRRNERLRRESVRPV

SEQ ID NO: 83 - protein, 88 aa, synthetic construct
  source 1..88: note = SLAMF7
WFLKRERQEEYIEEKKRVDICRETPNICPHSGENTEYDTIPHTNRTILKEDPANTVYSTVEIPKKMENPHSLLTMPDTPRLFAYENVI

SEQ ID NO: 84 - protein, 52 aa, synthetic construct
  source 1..52: note = DAP12
YFLGRLVPRGRGAAEAATRKQRITETESPYQELQGQRSDVYSDLNTQRPYYK

SEQ ID NO: 85 - protein, 46 aa, synthetic construct
  source 1..46: note = MRC1/CD206
YKKRRVHLPQEGAFENTLYFNSQSSPGTSDMKDLVGNIEQNEHSVI

SEQ ID NO: 86 - protein, 262 aa, synthetic construct
  source 1..262: note = MEGF10
YRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 87 - protein, 72 aa, synthetic construct
  source 1..72: note = STABILIN2
YSYFRINRRTIGFQHFESEEDINVAALGKQQPENISNPLYESTTSAPPEPSYDPFTDSEERQLEGNDPLRTL

SEQ ID NO: 88 - protein, 36 aa, synthetic construct
  source 1..36: note = BAI-1
YWSMRLQARGGPSPLKSNSDSARLPISSGSTSSSRI

SEQ ID NO: 89 - protein, 113 aa, synthetic construct
  source 1..113: note = CD3zeta
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 90 - protein, 55 aa, synthetic construct
  source 1..55: note = CD3epsilon
KNRKAKAKPVTRGAGAGGRQRGQNKERPPPVPNPDYEPIRKGQRDLYSGLNQRRI

SEQ ID NO: 91 - protein, 45 aa, synthetic construct
  source 1..45: note = CD3delta
GHETGRLSGAADTQALLRNDQVYQPLRDRDDAQYSHLGGNWARNK

SEQ ID NO: 92 - protein, 61 aa, synthetic construct
  source 1..61: note = FcgammaRI
RKELKRKKKWDLEISLDSGHEKKVISSLQEDRHLEEELKCQEQKEEQLQEGVHRKEPQGAT

SEQ ID NO: 93 - protein, 21 aa, synthetic construct
  source 1..21: note = T2A
GSGEGRGSLLTCGDVEENPGP

SEQ ID NO: 94 - protein, 22 aa, synthetic construct
  source 1..22: note = P2A
GSGATNFSLLKQAGDVEENPGP

SEQ ID NO: 95 - protein, 23 aa, synthetic construct
  source 1..23: note = E2A
GSGQCTNYALLKLAGDVESNPGP

SEQ ID NO: 96 - protein, 25 aa, synthetic construct
  source 1..25: note = F2A
GSGVKQTLNFDLLKLAGDVESNPGP

SEQ ID NO: 97 - protein, 503 aa, synthetic construct
  source 1..503: note = CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITKWKKKKRPRNSYKCGTNTMEREESEQTKKREKIHIPERSDEAQRVFKSSKTSSCDKSDTCFRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 98 - protein, 503 aa, synthetic construct; identical to SEQ ID NO: 97
  source 1..503: note = CAR

SEQ ID NO: 99 - protein, 478 aa, synthetic construct
  source 1..478: note = CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITCWWPSYLFQLGKAKKGNSKEDPQDKVTQIKLDDFTMRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 100 - protein, 503 aa, synthetic construct
  source 1..503: note = CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDVLLCLLLVPLLLSLFVLGLFLKWKKKKRPRNSYKCGTNTMEREESEQTKKREKIHIPERSDEAQRVFKSSKTSSCDKSDTCFRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 101 - protein, 509 aa, synthetic construct
  source 1..509: note = CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDWITAVLPTVIICVMVFCLILWWRRKRKEKQSETSPKEFLTIYEDVKDLKTRRNHEQEQTFPGGGSTIYSMIQSQSSAPTSTGGGSGGSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 102 - protein, 486 aa, synthetic construct
  source 1..486: note = CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDGLVVAIVLIVLCLIGLIVAILRWPPSAACSGKESVVAIRTNSQSDFHLQTYGDEDLNELDPHYEMRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 103 - protein, 463 aa, synthetic construct
  source 1..463: note = CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSALSNSIMYFSHFVPVFLPAKPTTTPAPRPPTPAPTIASQPLSLRPEACRPAAGGAVHTRGLDFACDIYIWAPLAGTCGVLLLSLVITRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 104 - protein, 524 aa, synthetic construct
  source 1..524: note = CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSALSNSIMYFSHFVPVFLPAKPTTTPAPRPPTPAPTIASQPLSLRPEACRPAAGGAVHTRGLDFACDIYIWAPLAGTCGVLLLSLVITKWKKKKRPRNSYKCGTNTMEREESEQTKKREKIHIPERSDEAQRVFKSSKTSSCDKSDTCFRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 105 - protein, 524 aa, synthetic construct; identical to SEQ ID NO: 104
  source 1..524: note = CAR

SEQ ID NO: 106 - protein, 524 aa, synthetic construct
  source 1..524: note = CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSALSNSIMYFSHFVPVFLPAKPTTTPAPRPPTPAPTIASQPLSLRPEACRPAAGGAVHTRGLDFACDVLLCLLLVPLLLSLFVLGLFLKWKKKKRPRNSYKCGTNTMEREESEQTKKREKIHIPERSDEAQRVFKSSKTSSCDKSDTCFRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 107 - protein, 467 aa, synthetic construct
  source 1..467: note = CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITTYCFAPRCRERRRNERLRRESVRPVRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 108 - protein, 468 aa, synthetic construct
  source 1..468: note = CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDVLLCLLLVPLLLSLFVLGLFLTTYCFAPRCRERRRNERLRRESVRPVRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 109 - protein, 467 aa, synthetic construct; identical to SEQ ID NO: 107
  source 1..467: note = CAR

SEQ ID NO: 110 - protein, 482 aa, synthetic construct
  source 1..482: note = CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITKWKKKKRPRNSYKCGTNTMEREESEQTKKREKIHIPERSDEAQRVFKSSKTSSCDKSDTCFRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 111 - protein, 482 aa, synthetic construct; identical to SEQ ID NO: 110
  source 1..482: note = CAR

SEQ ID NO: 112 - protein, 457 aa, synthetic construct
  source 1..457: note = CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITCWWPSYLFQLGKAKKGNSKEDPQDKVTQIKLDDFTMRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 113 - protein, 482 aa, synthetic construct
  source 1..482: note = CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDVLLCLLLVPLLLSLFVLGLFLKWKKKKRPRNSYKCGTNTMEREESEQTKKREKIHIPERSDEAQRVFKSSKTSSCDKSDTCFRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 114 - protein, 488 aa, synthetic construct
  source 1..488: note = CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDWITAVLPTVIICVMVFCLILWWRRKRKEKQSETSPKEFLTIYEDVKDLKTRRNHEQEQTFPGGGSTIYSMIQSQSSAPTSTGGGSGGSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 115 - protein, 465 aa, synthetic construct
  source 1..465: note = CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDGLVVAIVLIVLCLIGLIVAILRWPPSAACSGKESVVAIRTNSQSDFHLQTYGDEDLNELDPHYEMRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 116 - protein, 442 aa, synthetic construct
  source 1..442: note = CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSALSNSIMYFSHFVPVFLPAKPTTTPAPRPPTPAPTIASQPLSLRPEACRPAAGGAVHTRGLDFACDIYIWAPLAGTCGVLLLSLVITRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 117 - protein, 503 aa, synthetic construct
  source 1..503: note = CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSALSNSIMYFSHFVPVFLPAKPTTTPAPRPPTPAPTIASQPLSLRPEACRPAAGGAVHTRGLDFACDIYIWAPLAGTCGVLLLSLVITKWKKKKRPRNSYKCGTNTMEREESEQTKKREKIHIPERSDEAQRVFKSSKTSSCDKSDTCFRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 118 - protein, 503 aa, synthetic construct
  source 1..503: note = CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSALSNSIMYFSHFVPVFLPAKPTTTPAPRPPTPAPTIASQPLSLRPEACRPAAGGAVHTRGLDFACDVLLCLLLVPLLLSLFVLGLFLKWKKKKRPRNSYKCGTNTMEREESEQTKKREKIHIPERSDEAQRVFKSSKTSSCDKSDTCFRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 119 - protein, 446 aa, synthetic construct
  source 1..446: note = CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITTYCFAPRCRERRRNERLRRESVRPVRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 120 - protein, 447 aa, synthetic construct
  source 1..447: note = CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDVLLCLLLVPLLLSLFVLGLFLTTYCFAPRCRERRRNERLRRESVRPVRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 121 - protein, 446 aa, synthetic construct; identical to SEQ ID NO: 119
  source 1..446: note = CAR

SEQ ID NO: 122 - DNA, 276 bp, synthetic construct
  source 1..276: mol_type = other DNA; note = Synthetic promoter-146 (SP146)
ctagcgagggcggaccagaaaaggagaagtaggagccaagatttccaaactctgtggttgccttgccaagatttccaaactctgtggttgccttgcagaaaaggagaagtaggagaagcgacttcctctttccagaagcgacttcctctttccagaggaagagggcggaggctcacaaggcaaccacagagtttggaaatcttggaagcgacttcctctttccagcagaaaaggagaagtaggagaagcgacttcctctttccaggtccgccctcg

SEQ ID NO: 123 - DNA, 266 bp, synthetic construct
  source 1..266: mol_type = other DNA; note = Synthetic promoter-107 (SP107)
ctagcgagggcggacaagcgacttcctctttccagcagaaaaggagaagtaggagccaagatttccaaactctgtggttgccttgaagcgacttcctctttccagaagcgacttcctctttccagcagaaaaggagaagtaggagctcctacttctccttttctgccaagatttccaaactctgtggttgccttgctcctacttctccttttctgcagaaaaggagaagtaggagctggaaagaggaagtcgcttgagggcggacg

SEQ ID NO: 124 - DNA, 176 bp, synthetic construct
  source 1..176: mol_type = other DNA; note = Synthetic promoter-60 (SP60)
ctagcgtccgccctcaggaagagggcggaggctcaccaagatttccaaactctgtggttgccttgccaagatttccaaactctgtggttgccttgccaagatttccaaactctgtggttgccttgtgagcctccgccctcttcctaagcgacttcctctttccaggagggcggacg

SEQ ID NO: 125 - protein, 442 aa, synthetic construct
  source 1..442: note = CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 126 - protein, 443 aa, synthetic construct
  source 1..443: note = CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDALVVIPIIFGILFAILLVLVFIRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 127 - protein, 442 aa, synthetic construct
  source 1..442: note = CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDVVVACMSIMALLLLLLLLLLYRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 128 - protein, 421 aa, synthetic construct
  source 1..421: note = CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 129 - protein, 422 aa, synthetic construct
  source 1..422: note = CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDALVVIPIIFGILFAILLVLVFIRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 130 - protein, 421 aa, synthetic construct
  source 1..421: note = CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDVVVACMSIMALLLLLLLLLLYRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 131 - protein, 486 aa, synthetic construct; identical to SEQ ID NO: 102
  source 1..486: note = CAR

SEQ ID NO: 132 - protein, 465 aa, synthetic construct; identical to SEQ ID NO: 115
  source 1..465: note = CAR

(19) United States
(12) Patent Application Publication
(10) Pub. No.: US 2026/0356936 A1
(43) Pub. Date: Nov. 12, 2026

(54) METHODS AND COMPOSITIONS FOR GENERATING CHIMERIC ANTIGEN RECEPTOR-MONOCYTES/MACROPHAGES

(71) Applicant: Shoreline Biosciences, Inc., San Diego, CA (US)

(72) Inventors: Christine HUH, San Diego, CA (US); Fereshteh PARVIZ, San Diego, CA (US); David RODGERS, San Diego, CA (US); May SUMI, San Diego, CA (US); Matthew THAYER, San Diego, CA (US); Huafeng WANG, San Diego, CA (US)

(73) Assignee: Shoreline Biosciences, Inc., San Diego, CA (US)

(21) Appl. No.: 19/099,308

(22) PCT Filed: Jul. 28, 2023
(86) PCT No.: PCT/US2023/071207

§ 371 Date: Jan. 28, 2025

Related U.S. Application Data
(60) Provisional application No. 63/393,710, filed on Jul. 29, 2022.
(60) Provisional application No. 63/430,316, filed on Dec. 5, 2022.

(51) Int. Cl. C07K 16/28; A61K 40/17; A61K 40/31; A61K 40/42; A61P 35/00; C07K 14/705; C12N 5/0786; C12N 15/86

(52) U.S. Cl. CPC C07K 16/2803; A61K 40/17; A61K 40/31; A61K 40/4211; A61P 35/00; C07K 14/70503; C07K 14/7051; C07K 14/70517; C12N 5/0645; C12N 15/86; C07K 2317/622; C07K 2319/02; C07K 2319/03; C12N 2510/00; C12N 2740/15043

(57) ABSTRACT

The present disclosure relates, in part, to methods and compositions for generating monocytes, macrophages, and/or precursors thereof (such as an induced pluripotent stem cell), comprising (i) a chimeric antigen receptor (CAR); (ii) a polynucleotide encoding a CAR; (iii) a CAR polypeptide; and/or (iv) a vector comprising a polynucleotide encoding a CAR. Methods for treating a cancer using cells expressing the CAR are also provided herein. In some embodiments, the cells expressing the CAR comprise a genetic disruption of a SIRPA gene, CISH gene, and/or SIGLEC10 gene.

### CROSS REFERENCE

This application claims the benefit of U.S. Provisional Application No. 63/393,710, filed Jul. 29, 2022, and U.S. Provisional Application No. 63/430,316, filed Dec. 5, 2023, the entirety of which are hereby incorporated by reference herein in their entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14735-014-228_SEQ_LISTING.xml,” was created on Jul. 27, 2023, and is 159,867 bytes in size.

### 1. FIELD

The present disclosure relates, in part, to methods and compositions for generating monocytes, macrophages, and/or precursors thereof (such as an induced pluripotent stem cell), comprising (i) a chimeric antigen receptor (CAR); (ii) a polynucleotide encoding a CAR; (iii) a CAR polypeptide; and/or (iv) a vector comprising a polynucleotide encoding a CAR. In some embodiments, the cells expressing the CAR comprise a genetic disruption of a SIRPA gene, a CISH gene, and/or a SIGLEC10 gene.

### 2. BACKGROUND

Chimeric antigen receptor (CAR) T cell therapy is an effective treatment for certain types of hematological cancers, such as B cell leukemia and lymphoma. However, the therapeutic efficacy of CAR-T cells in solid tumors remains a challenge due, in part, to limited tumor infiltration of T cells. Macrophages are the innate immune cells with the highest infiltration rate. Yet, macrophages are often unable to identify and attack tumors, and instead behave in an immunosuppressive manner. Modification of the macrophages by introducing a CAR may allow these infiltrating cells to better reduce or eliminate cancer cells.

Current CAR macrophage approaches involve constructs based on CAR T cells despite differences between the signaling pathways in T cells and macrophages/monocytes. In addition, macrophages present unique manufacturing challenges, such as the ability to expand a sufficient amount of cells, and to do so in a timely manner that is not cost-prohibitive. Although macrophages can be derived from monocytes, even monocyte-derived macrophages have barriers for expansion, which limits the speed and scalability of manufacturing. Thus, there is an unmet need to develop improved CAR macrophages/monocytes that are specifically designed for macrophages/monocytes, and also develop a manufacturing system to addresses the challenges of working with macrophages/monocytes.

### 3. SUMMARY

In one aspect, provided herein is a polynucleotide encoding a chimeric antigen receptor (CAR), wherein the CAR comprises an antigen recognition moiety, a hinge domain, a transmembrane domain, and an intracellular domain comprising: (a) a non-lymphoid intracellular signaling domain; and (b) a CD3, intracellular signaling domain, and wherein the polynucleotide is operatively linked to a promoter.

In one embodiment of the polynucleotide provided herein, the promoter is a myeloid-specific promoter. In a certain embodiment provided herein, the myeloid-specific promoter is a native macrophage or a native monocyte promoter. In another embodiment, the myeloid-specific promoter is a synthetic promoter. In one embodiment provided herein, the synthetic promoter is selected from the group consisting of synthetic promoter-146 (SP146), synthetic promoter-107 (SP107), and synthetic promoter-60 (SP60).

In one embodiment of the polynucleotide provided herein, the promoter is selected from the group consisting of a EF1α, CD36, CD68, SP60, SP107, SP146, CAG, PGK, T7, and CMV promoter.

In one embodiment of the polynucleotide provided herein, the antigen recognition moiety comprises a scFv antigen recognition moiety. In one aspect, the scFv antigen recognition moiety recognizes a tumor antigen or fragment thereof. In a certain embodiment provided herein, the tumor antigen is selected from the group consisting of HER2/neu, PSMA, Claudin18, CD20, CD5, mesothelin, GD2, and CD19.

In one embodiment of the polynucleotide provided herein, the hinge domain comprises a CD8 hinge domain.

In one embodiment of the polynucleotide provided herein, the transmembrane domain enhances activity of a CAR in a macrophage relative to the CAR having a transmembrane domain consisting of a CD8 transmembrane domain. In an embodiment provided herein, the increase in activity comprises an increase in secretion of a pro-inflammatory cytokine. In another embodiment, the increase in secretion of the pro-inflammatory cytokine is about 10-fold to about 100-fold. In certain embodiment provided herein, the increase in secretion of the pro-inflammatory cytokine is about 25-fold. In one or more embodiments provided herein, the increase in secretion of the pro-inflammatory cytokine is about 50-fold, about 75-fold, about 100-fold or more than about 100-fold.

In one embodiment of the polynucleotide provided herein, the pro-inflammatory cytokine is selected from the group consisting of CXCL10, CCL1, CCL2, CCL22, MIP-1α/CCL3, IL-10, IL-1β, IFNγ, IL-6, TNFA, and CCL20, or a combination thereof. In a certain embodiment, the pro-inflammatory cytokine comprises MIP-1α/CCL3. In one embodiment provided herein, the increase in secretion of the pro-inflammatory cytokine comprises a composite score of two of more cytokines. In a certain embodiment provided herein, the secretion of the pro-inflammatory cytokine is measured by ELISA after culturing the macrophage for 24 hours in the presence of a target antigen. In one embodiment provided herein, the increase in activity comprises an increase in macrophage phagocytosis. In certain embodiment, the increase in macrophage phagocytosis is about 10% to about 50%. In other embodiments, the increase is about 10%, the increase is about 20%, the increase is about 30%, the increase is about 40%, or the increase is more than about 40%.

In one embodiment of the polynucleotide provided herein, phagocytosis is measured by flow cytometry after about four hours of co-incubation of a macrophage expressing the CAR and a target cell expressing a target antigen.

In one aspect of the polynucleotide provided herein, the transmembrane domain is selected from the group consisting of IL-1R1, VEGFR2, IFNγR1, CSF3R, CSF2R, SLAMF7, Dectin-1, Dectin-3, TLR4, CSF1R, CD8, CD28, MINCLE, MDL1, BDCA2, DAP12, CD11b, CD86, and CD40. In some embodiments, the transmembrane domain is not CD8. In some embodiments, the transmembrane domain is not TLR4. In some embodiments, the transmembrane domain is not CD28. In some embodiments, the transmembrane domain is not CD86.

In one embodiment of the polynucleotide provided herein, the intracellular domain increases activity of a CAR in a macrophage relative to the CAR having an intracellular domain consisting of a CD3ζ intracellular signaling domain. In another aspect, the increase in activity comprises an increase in secretion of a pro-inflammatory cytokine. In one aspect provided herein, the increase in secretion of the pro-inflammatory cytokine is about 10-fold to about 100-fold. In other embodiments provided herein, the increase in secretion of the pro-inflammatory cytokine is about 25-fold, about 50-fold, about 75-fold, about 100-fold or more than about 100-fold.

In one embodiment of the polynucleotide provided herein, the one or more non-lymphoid intracellular signaling domains are selected from the group consisting of BAI-1, CD86/B7-2, Lox1c, TM4, MEGF10, SCARF1, CD93, DAP12, SLAMF7, IFNγR2, 2B4/CD244, Dectin-1, CD206, Dectin-3, CLEC2, and CD80/B7-1.

In one aspect, provided herein is a polynucleotide encoding a chimeric antigen receptor (CAR), wherein the CAR comprises
(a) an antigen recognition moiety;
(b) a CD8 hinge domain,
(c) a transmembrane domain, wherein the transmembrane domain is selected from the group consisting of (i) a CD8 transmembrane domain; (ii) a SLAMF7 transmembrane domain; (iii) a CD86 transmembrane domain; (iv) a Dectin-1 transmembrane domain; and (v) a
(d) a first intracellular signaling domain, wherein the first intracellular signaling domain is selected from the group consisting of (i) a CD86 intracellular signaling domain; (ii) a Lox1c intracellular signaling domain, (iii) a 2B4 intracellular signaling domain; (iv) a Dectin-1 intracellular signaling domain; and (v) a CD40 intracellular signaling domain; and
(e) a second intracellular domain comprising a CD3ζ intracellular signaling domain,
wherein the polynucleotide is operatively linked to a promoter.

In one aspect of the polynucleotide provided herein, the polynucleotide encodes for a hinge domain comprising an amino acid sequence of SEQ ID NO:1.

In one embodiment of the polynucleotide provided herein, the transmembrane domain comprises a CD8 transmembrane domain.

In one embodiment of the polynucleotide provided herein, the first intracellular signaling domain comprises a CD86 intracellular signaling domain.

In one embodiment of the polynucleotide provided herein, the first intracellular signaling domain comprises a Lox1c intracellular signaling domain.

In one embodiment of the polynucleotide provided herein, the transmembrane domain comprises a SLAMF7 transmembrane domain.

In one embodiment of the polynucleotide provided herein, the first intracellular signaling domain comprises a CD86 intracellular signaling domain.

In one embodiment of the polynucleotide provided herein, the transmembrane domain comprises a CD86 transmembrane domain.

In one embodiment of the polynucleotide provided herein, the first intracellular signaling domain comprises a 2B4 intracellular signaling domain.

In one embodiment of the polynucleotide provided herein, the transmembrane domain comprises a Dectin-1 transmembrane domain.

In one embodiment of the polynucleotide provided herein, the transmembrane domain comprises a CSF1R transmembrane domain.

In one embodiment of the polynucleotide provided herein, the first intracellular signaling domain comprises a Dectin-1 intracellular signaling domain.

In one embodiment of the polynucleotide provided herein, the first intracellular signaling domain comprises a CD40 intracellular signaling domain.

In one embodiment of the polynucleotide provided herein, the polynucleotide encodes for a hinge domain comprising an amino acid sequence of SEQ ID NO:4.

In one embodiment of the polynucleotide provided herein, the first intracellular signaling domain comprises a CD86 intracellular signaling domain.

In one embodiment of the polynucleotide provided herein, the transmembrane domain comprises a SLAMF7 transmembrane domain.

In one embodiment of the polynucleotide provided herein, the promoter is a constitutive promoter. In one embodiment, the constitutive promoter is a EF1α promoter. In another embodiment, the promoter is a myeloid-specific promoter. In one embodiment provided herein, the myeloid-specific promoter is a native macrophage promoter or a native monocyte promoter. In another embodiment, the myeloid-specific promoter is a synthetic promoter. In one embodiment, the synthetic promoter is selected from the group consisting of synthetic promoter-146 (SP146), synthetic promoter-107 (SP107), and synthetic promoter-60 (SP60). In a specific embodiment, the synthetic promoter is SP146.

In one embodiment of the polynucleotide provided herein, the antigen recognition moiety comprises a scFv antigen recognition moiety. In a certain embodiment, the scFv antigen recognition moiety recognizes a tumor antigen or fragment thereof. In another embodiment, the tumor antigen is selected from your group consisting of HER2/neu, PSMA, Claudin18, CD20, CD5, mesothelin, GD2, and CD19.

Provided herein is a vector comprising the polynucleotide of any one of embodiments disclosed herein.

In one embodiment of the vector provided herein, the vector comprises DNA or RNA. In another embodiment, the vector is a plasmid vector. In a certain embodiment, the vector is a viral vector. In an embodiment provided herein, the viral vector is selected from the group consisting of an adenoviral vector, a lentiviral vector, and a retroviral vector. In a specific embodiment, the viral vector is a lentiviral vector.

In one aspect, provided herein is a polypeptide comprising:
(a) an antigen recognition moiety polypeptide;
(b) a CD8 hinge domain polypeptide;
(c) a transmembrane domain polypeptide, wherein the transmembrane domain is selected from the group consisting of (i) a CD8 transmembrane domain; (ii) a SLAMF7 transmembrane domain; (iii) a CD86 transmembrane domain; (iv) a Dectin-1 transmembrane domain; and (v) a CSF1R transmembrane domain;
(d) a first intracellular signaling domain polypeptide, wherein the first intracellular signaling domain is selected from the group consisting of: (i) a CD86 intracellular signaling domain; (ii) a Lox1c intracellular signaling domain, (iii) a 2B4 intracellular signaling domain; (iv) a Dectin-1 intracellular signaling domain; and (v) a CD40 intracellular signaling domain; and
(e) a second intracellular domain polypeptide comprising a CD3ζ intracellular signaling domain.

In an embodiment of the polypeptide provided herein, the hinge domain has an amino acid sequence of SEQ ID NO:1.

In an embodiment of the polypeptide provided herein, the transmembrane domain polypeptide comprises a CD8 transmembrane domain polypeptide. In one aspect, the CD8 transmembrane domain polypeptide has an amino acid sequence of SEQ ID NO:11.

In one embodiment of the polypeptide provided herein, the first intracellular signaling domain polypeptide comprises a CD86 intracellular signaling domain polypeptide. In one aspect, the CD86 intracellular signaling domain polypeptide has an amino acid sequence of SEQ ID NO: 40.

In one embodiment of the polypeptide provided herein, the first intracellular signaling domain polypeptide comprises a Lox1c intracellular signaling domain polypeptide. In one embodiment, the Lox1c intracellular signaling domain polypeptide has an amino acid sequence of SEQ ID NO:41.

In one embodiment of the polypeptide provided herein, the transmembrane domain polypeptide comprises a SLAMF7 transmembrane domain polypeptide. In a specific embodiment, the SLAMF7 transmembrane domain polypeptide has an amino acid sequence of SEQ ID NO:12.

In one embodiment of the polypeptide provided herein, the first intracellular signaling domain polypeptide comprises a CD86 intracellular signaling domain polypeptide. In a specific embodiment, the CD86 intracellular signaling domain polypeptide has an amino acid sequence of SEQ ID NO:40.

In one embodiment of the polypeptide provided herein, the transmembrane domain polypeptide comprises a CD86 transmembrane domain polypeptide. In a specific embodiment, the CD86 transmembrane domain polypeptide has an amino acid sequence of SEQ ID NO: 13.

In one embodiment of the polypeptide provided herein, the first intracellular signaling domain polypeptide comprises a 2B4 intracellular signaling domain polypeptide. In a specific embodiment, the 2B4 intracellular signaling domain polypeptide has an amino acid sequence of SEQ ID NO:42.

In one embodiment of the polypeptide provided herein, the transmembrane domain polypeptide comprises a Dectin-1 transmembrane domain polypeptide. In a specific embodiment, the Dectin-1 transmembrane domain polypeptide has an amino acid sequence of SEQ ID NO:14.

In one embodiment of the polypeptide provided herein, the transmembrane domain polypeptide comprises a CSF1R transmembrane domain polypeptide. In a specific embodiment, the CSF1R transmembrane domain polypeptide has an amino acid sequence of SEQ ID NO:33.

In one embodiment of the polypeptide provided herein, the first intracellular signaling domain polypeptide comprises a Dectin-1 intracellular signaling domain polypeptide. In a specific embodiment, the Dectin-1 intracellular signaling domain polypeptide has an amino acid sequence of SEQ ID NO:43.

In one embodiment of the polypeptide provided herein, the first intracellular signaling domain polypeptide comprises a CD40 intracellular signaling domain polypeptide. In a specific embodiment, the CD40 intracellular signaling domain polypeptide has an amino acid sequence of SEQ ID NO:46.

In one embodiment of the polypeptide provided herein, the hinge domain polypeptide comprises SEQ ID NO:4.

In one embodiment of the polypeptide provided herein, the first intracellular signaling domain polypeptide comprises a CD86 intracellular signaling domain polypeptide. In a specific embodiment, the CD86 intracellular signaling domain polypeptide has an amino acid sequence of SEQ ID NO:40.

In one embodiment of the polypeptide provided herein, the transmembrane domain polypeptide comprises a SLAMF7 transmembrane domain. In a specific embodiment, the SLAMF7 transmembrane domain polypeptide has an amino acid sequence of SEQ ID NO:12.

In one embodiment of the polypeptide provided herein, the antigen recognition moiety polypeptide comprises a scFv antigen recognition moiety polypeptide. In one embodiment, the scFv antigen recognition moiety polypeptide recognizes a tumor antigen or fragment thereof. In a certain embodiment, the tumor antigen is selected from your group consisting of HER2/neu, PSMA, Claudin18, CD20, CD5, mesothelin, GD2, and CD19.

In one aspect, provided herein is a polypeptide having an amino acid sequence comprising or consisting of SEQ ID NO:97.

In one aspect, provided herein is a polypeptide having an amino acid sequence comprising or consisting of SEQ ID NO:98.

In one aspect, provided herein is a polypeptide having an amino acid sequence comprising or consisting of SEQ ID NO:99.

In one aspect, provided herein is a polypeptide having an amino acid sequence comprising or consisting of SEQ ID NO:100.

In one aspect, provided herein is a polypeptide having an amino acid sequence comprising or consisting of SEQ ID NO:101.

In one aspect, provided herein is a polypeptide having an amino acid sequence comprising or consisting of SEQ ID NO:102.

In one aspect, provided herein is a polypeptide having an amino acid sequence comprising or consisting of SEQ ID NO: 103.

In one aspect, provided herein is a polypeptide having an amino acid sequence comprising or consisting of SEQ ID NO:104.

In one aspect, provided herein is a polypeptide having an amino acid sequence comprising or consisting of SEQ ID NO:105.

In one aspect, provided herein is a polypeptide having an amino acid sequence comprising or consisting of SEQ ID NO:106.

In one aspect, provided herein is a polypeptide having an amino acid sequence comprising or consisting of SEQ ID NO:107.

In one aspect, provided herein is a polypeptide having an amino acid sequence comprising or consisting of SEQ ID NO:108.

In one aspect, provided herein is a polypeptide having an amino acid sequence comprising or consisting of SEQ ID NO:110.

In one aspect, provided herein is a polypeptide having an amino acid sequence comprising or consisting of SEQ ID NO:111.

In one aspect, provided herein is a polypeptide having an amino acid sequence comprising or consisting of SEQ ID NO:112.

In one aspect, provided herein is a polypeptide having an amino acid sequence comprising or consisting of SEQ ID NO:113.

In one aspect, provided herein is a polypeptide having an amino acid sequence comprising or consisting of SEQ ID NO:114.

In one aspect, provided herein is a polypeptide having an amino acid sequence comprising or consisting of SEQ ID NO:115.

In one aspect, provided herein is a polypeptide having an amino acid sequence comprising or consisting of SEQ ID NO:116.

In one aspect, provided herein is a polypeptide having an amino acid sequence comprising or consisting of SEQ ID NO:117.

In one aspect, provided herein is a polypeptide having an amino acid sequence comprising or consisting of SEQ ID NO:118.

In one aspect, provided herein is a polypeptide having an amino acid sequence comprising or consisting of SEQ ID NO: 119.

In one aspect, provided herein is a polypeptide having an amino acid sequence comprising or consisting of SEQ ID NO:120.

In one aspect, provided herein is a polypeptide having an amino acid sequence comprising or consisting of SEQ ID NO: 121.

In one aspect, provided herein is a polypeptide having an amino acid sequence comprising or consisting of SEQ ID NO:125.

In one aspect, provided herein is a polypeptide having an amino acid sequence comprising or consisting of SEQ ID NO:126.

In one aspect, provided herein is a polypeptide having an amino acid sequence comprising or consisting of SEQ ID NO:127.

In one aspect, provided herein is a polypeptide having an amino acid sequence comprising or consisting of SEQ ID NO:128.

In one aspect, provided herein is a polypeptide having an amino acid sequence comprising or consisting of SEQ ID NO:129.

In one aspect, provided herein is a polypeptide having an amino acid sequence comprising or consisting of SEQ ID NO:130.

In one aspect, provided herein is a polypeptide having an amino acid sequence comprising or consisting of SEQ ID NO: 131.

In one aspect, provided herein is a polypeptide having an amino acid sequence comprising or consisting of SEQ ID NO:132.

In one aspect, provided herein is a modified cell, wherein the modified cell comprises the polynucleotide of any one of the embodiments disclosed herein, the vector of any one of embodiments disclosed herein, or the polypeptide of any one of embodiments disclosed herein, and wherein the modified cell is a monocyte, a macrophage, or an induced pluripotent stem cell (iPSC). In one aspect of the modified cell provided herein, the modified cell is an iPSC. In another aspect, the modified cell is a macrophage. In one embodiment, the modified cell is a monocyte. In another embodiment, the modified cell is derived from an iPSC. In one aspect of the modified cell provided herein, the modified cell is a human cell. In some embodiments, the modified cell comprises a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the modified cell comprises genetic disruption of SIRPA. In some embodiments, the modified cell comprises genetic disruption of CISH. In some embodiments, the modified cell comprises genetic disruption of SIGLEC10.

In some embodiments, the modified cell provided herein comprises an agent capable of preventing or reducing interaction between CD47 and SIRPα. In some embodiments, the agent comprises an anti-CD47 antibody. In some embodiments, the agent comprises an anti-SIRPα antibody.

In some embodiments, the modified cell provided herein comprises an agent capable of preventing or reducing interaction between CD24 and SIGLEC10. In some embodiments, the agent comprises an anti-CD24 antibody. In some embodiments, the agent comprises an anti-SIGLEC10 antibody.

In one embodiment, provided herein is a pharmaceutical composition comprising the modified cell of any one of embodiments disclosed herein.

In one embodiment, provided herein is a method of treating a cancer, comprising administering the pharmaceutical composition disclosed herein.

In one embodiment, provided herein is a method of engineering a modified cell, comprising introducing into the cell the polynucleotide of any one of embodiments disclosed herein, the vector of any one of embodiments disclosed herein, or the polypeptide of any one of embodiments disclosed herein, wherein the cell is a monocyte, a macrophage, or an induced pluripotent stem cell (iPSC). In one aspect of the method provided herein, the cell is an iPSC. In another aspect, the cell is a macrophage. In one embodiment, the cell is a monocyte. In another embodiment of the method provided herein, the cell is derived from an iPSC. In one aspect of the method provided herein, the modified cell is a human cell. In some embodiments, the cell comprises a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the modified cell comprises genetic disruption of SIRPA. In some embodiments, the modified cell comprises genetic disruption of CISH. In some embodiments, the modified cell comprises genetic disruption of SIGLEC10.

### 4. BRIEF DESCRIPTION OF THE DRAWINGS

FIG. 1A-FIG. 1B. illustrate exemplary assays for measuring phagocytosis (FIG. 1A) and cytokine secretion (FIG. 1B).

FIG. 2A-FIG. 2B illustrate results from screening promoters for myeloid specific CAR expression in human THP1 macrophages using an anti-idiotype PE antibody against the CAR scFv. The promoters are listed on the left of the graphical representation in the order in which their respective data is presented. Expression was measured by flow cytometry (FIG. 2A), and quantified by mean fluorescent intensity (MFI) (FIG. 2B).

FIG. 3 illustrates results from an exemplary phagocytosis assay in human THP1 macrophages expressing CARs that were substituted with a series of transmembrane (TM) domains from receptors associated with macrophage activity, and normalized to THP1 macrophages expressing an otherwise identical CAR having a CD8 TM domain (“control”).

FIG. 4 illustrates results from an exemplary cytokine assay measuring macrophage inflammatory protein-1 alpha (MIP-1α)/CCL3 secretion in human THP1 macrophages after expressing CARs substituted with a series of transmembrane (TM) domains from receptors associated with macrophage activity, and normalized to THP1 macrophages expressing an otherwise identical CAR having a CD8 TM domain (“control”).

FIG. 5 illustrates results from an exemplary phagocytosis assay in human THP1 macrophages expressing CARs with a CD3ζ intracellular domain that were complimented with a series of additional intracellular domains from receptors associated with macrophage activity, and normalized to THP1 macrophages expressing an otherwise identical CAR where the intracellular domain was only a CD3ζ intracellular domain (“control”).

FIG. 6 illustrates results from an exemplary cytokine secretion assay measuring MIP-1α/CCL3 secretion in human THP1 macrophages expressing CARs with a CD3ζ intracellular domain that were complimented with a series of additional intracellular domains from receptors associated with macrophage activity, and normalized to THP1 macrophages expressing an otherwise identical CAR where the intracellular domain was only a CD3ζ intracellular domain (“control”).

FIG. 7 illustrates exemplary CAR constructs. Abbreviations: HD=hinge domain; TM=transmembrane domain; IC=intracellular domain; Abx=antibiotic.

FIG. 8 illustrates a western blot confirming SIRPA knockout in monocytes 17 days and 20 days after differentiation of SIRPA KO iPSCs. Monocytes derived from TC-1133 iPSCs that express SIRPA were wild-type control cells. Vinculin served as the housekeeping gene control.

FIG. 9-FIG. 10 illustrate flow cytometry data for iPSC derived SIRPA KO monocytes at Day 17 (FIG. 9) and Day 20 (FIG. 10) relative to wild-type control monocytes derived from TC-1133 iPSCs that express SIRPA. Live singlets were gated on CD45+ and then CD11b+CD14+ to confirm monocyte purity.

FIG. 11A-FIG. 11C illustrate flow cytometry data for SIRPα expression using an antibody that detects SIRPα surface expression (FIG. 11B) or intracellular expression (FIG. 11C) in iPSC-derived SIRPA KO macrophages obtained from D17 or D20 iPSC-derived monocytes, relative to wild-type control monocytes derived from TC-1133 iPSCs that express SIRPA.

FIG. 12A and FIG. 12B illustrate that SIRPA KO monocytes derived from SIRPA KO iPSCs have increased killing on Raji cells relative to wild-type control monocytes derived from TC-1133 iPSCs that express SIRPA, with the addition of 0.3 μg/mL and 0.5 μg/mL rituximab.

FIG. 13A and FIG. 13B illustrates that SIRPA KO macrophages derived from SIRPA KO iPSCs have increased killing relative to wild-type control macrophages derived from TC-1133 iPSCs that express SIRPA, with the addition of 0.1 μg/mL and 0.3 μg/mL rituximab in Raji cells (FIG. 13A), and with the addition of 10 μg/mL trastuzumab in BT474 cells FIG. 13B).

FIG. 14A and FIG. 14B illustrate that SIRPA KO macrophages derived from iPSCs have increased killing on Raji cells relative to wild-type control macrophages derived from TC-1133 iPSCs that express SIRPA, with the addition of 0.1 μg/mL and 0.3 μg/mL rituximab.

FIG. 15A and FIG. 15B illustrate a schema of iMACs pre-complexed with rituximab (RTX) (FIG. 15A) and in vivo results that demonstrated the iMACs pre-complexed with rituximab (RTX) kill Raji cells (FIG. 15B).

FIG. 16A-FIG. 16F illustrate CD19 (top panel) and red fluorescent protein (RFP) (bottom panel) expression, as measured by flow cytometry, post knock-in of CAR29 (FIG. 16A), CAR30 (FIG. 16B), CAR31 (FIG. 16C), CAR32 (FIG. 16D), CAR33 (FIG. 16E), and CAR34 (FIG. 16F) in iMACs.

FIG. 17A and FIG. 17B illustrate iMAC CAR function for CAR29-CAR34 according to tumor cell killing against CD19+Raji cells over 96 hours at a 5:1 effector:target ratio, without (FIG. 17A) or with (FIG. 17B) rituximab (anti-CD20).

FIG. 18A-FIG. 18B illustrate CD19 (top panel) and red fluorescent protein (RFP) (bottom panel) expression, as measured by flow cytometry, post knock-in of CAR31 (FIG. 18A), and CAR32 (FIG. 18B) in iMAC.

FIG. 19 illustrates iMAC CAR function for CAR31, and CAR32 according to tumor cell killing against CD19+Raji cells over 96 hours at a 5:1 effector:target ratio.

FIG. 20A-FIG. 20C illustrate viability for SirpaKO iMonocyte on day 20, as measured by flow cytometry (FIG. 20A); CD19 expression 24 hour post-electroporation for SirpaKO iMonocyte on day 20, as measured by flow cytometry (FIG. 20B); and SirpaKO iMAC CAR function for CAR25, CAR26, CAR27, and CAR28, or SirpaKO iMAC cells with 1 μg/mL rituximab (Rtx) according to tumor cell killing against CD19+Raji cells over 96 hours at a 5:1 effector:target ratio.

### 5. DETAILED DESCRIPTION

The present disclosure is directed, in part, to methods and compositions for generating monocytes, macrophages, and/or precursors thereof (such as an induced pluripotent stem cell) comprising (i) a chimeric antigen receptor (CAR); (ii) a polynucleotide encoding a CAR; (iii) a CAR polypeptide; and/or (iv) a vector comprising a polynucleotide encoding a CAR. Also provided herein, are methods of treating a disease or disorder comprising administering a pharmaceutical composition (such as a pharmaceutical composition provided herein). In some embodiments, provided herein is a method for treating cancer in a subject in need thereof, comprising administering a pharmaceutical composition (such as a pharmaceutical composition provided herein).

Unless defined otherwise, all technical and scientific terms used herein have the same meaning as commonly understood to one of ordinary skill in the art to which the invention pertains. Otherwise, certain terms used herein have the meanings as set forth in the specification. All patents, published patent applications and publications cited herein are incorporated by reference as if set forth fully herein. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

As used herein, the abbreviations for the genetically encoded amino acids are conventional and are as follows:

| Amino Acid | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

It should be noted that as used herein and in the appended claims, the singular forms “a,” “an,” and “the” include plural reference unless the context clearly dictates otherwise.

The term “percent identity” as used herein has the same meaning as commonly understood to one of ordinary skill in the art. A representative way to determine percent identity is by a direct comparison of the sequence information between two molecules by aligning the sequences, counting the exact number of matches between the two aligned sequences, dividing by the length of the shorter sequence, and multiplying the result by 100.

As used herein, the terms “about” and “approximately” mean within 20%, within 15%, within 10%, within 9%, within 8%, within 7%, within 6%, within 5%, within 4%, within 3%, within 2%, within 1%, or less of a given value or range.

As used herein the term “non-lymphoid intracellular domain” is intended to mean a signaling or costimulatory domain that is not part of the native T cell receptor complex (e.g., CD3ζ, CD3ζ, or CD3ε) native B cell receptor complex (e.g. CD22, CD79a, CD79b), or involved in the classical T cell signaling pathway, such as a co-stimulatory molecule from the CD28 family (e.g., CD28 or ICOS) or tumor necrosis factor receptor (TNFR) family (e.g., 4-1BB, OX40, or CD27).

As used herein, the term “myeloid specific promoter” is intended to mean a promoter that enhances or facilitates transcription of a gene in a myeloid cell (e.g., a macrophage or monocyte), relative to a non-myeloid cell as measured by, for example, a reporter gene assay (e.g., a luciferase, or chemiluminescent reporter assay) or measure of the downstream protein encoded by the gene. A representative non-myeloid reference cell may be a HeLa or a 293T cell.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Glover, ed., DNA Cloning, Volumes I and II (1985); Freshney, ed., Animal Cell Culture: Immobilized Cells and Enzymes (IRL Press, 1986); Immunochemical Methods in Cell and Molecular Biology (Academic Press, London); Scopes, Protein Purification: Principles and Practice (Springer Verlag, N.Y., 2d ed. 1987); and/or Janeway, et al. Immunobiology (Garland Science, 7th ed. 2008).

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. The application contemplates use of any of the applicable components in any combination, whether or not a particular combination is expressly described.

### 5.1. Chimeric Antigen Receptors

As provided herein, the present disclosure relates to chimeric antigen receptors (CARs) that activate monocyte/macrophage activity. In some embodiments, a CAR provided herein comprises an antigen recognition moiety (such as an antigen recognition moiety described in Section 5.1.1), a hinge domain (such as a hinge domain described in Section 5.1.2), a transmembrane domain (such as a transmembrane domain described in Section 5.1.3), and an intracellular domain (such as an intracellular domain described in Section 5.1.4), and the transmembrane domain and/or intracellular domain comprise domains derived from a receptor associated with macrophage activity. In some embodiments, a CAR provided herein comprises an antigen recognition moiety (such as an antigen recognition moiety described in Section 5.1.1), a hinge domain (such as a hinge domain described in Section 5.1.2), a transmembrane domain (such as a transmembrane domain described in Section 5.1.3), and an intracellular domain (such as an intracellular domain described in Section 5.1.4), and the transmembrane domain comprises a transmembrane domain derived from a receptor associated with macrophage activity. In some embodiments, a CAR provided herein comprises an antigen recognition moiety (such as an antigen recognition moiety described in Section 5.1.1), a hinge domain (such as a hinge domain described in Section 5.1.2), a transmembrane domain (such as a transmembrane domain described in Section 5.1.3), and an intracellular domain (such as an intracellular domain described in Section 5.1.4), and the intracellular domain comprises an intracellular domain derived from a receptor associated with macrophage activity. In some embodiments, a CAR provided herein comprises an antigen recognition moiety (such as an antigen recognition moiety described in Section 5.1.1), a hinge domain (such as a hinge domain described in Section 5.1.2), a transmembrane domain (such as a transmembrane domain described in Section 5.1.3), and an intracellular domain (such as an intracellular domain described in Section 5.1.4), and both the transmembrane domain and intracellular domain comprise domains derived from a receptor associated with macrophage activity. In some embodiments, the macrophage activity comprises secretion of a pro-inflammatory cytokine. In some embodiments, the macrophage activity comprises phagocytosis of a cell expressing a target antigen. In some embodiments, the macrophage activity comprises secretion of a pro-inflammatory cytokine and phagocytosis of a cell expressing a target antigen.

In some aspects, the chimeric antigen receptor (CAR) of the present disclosure comprises a signal peptide. In some embodiments, the signal peptide has an amino acid sequence of MALPVTALLLPLALLLHAARP (SEQ ID NO: 2). In some embodiments, the signal peptide has an amino acid sequence with at least 80% sequence identity with SEQ ID NO: 2. In some embodiments, the signal peptide has an amino acid sequence with at least 90%, 95%, 97% or 99% sequence identity with SEQ ID NO: 2.

In some embodiments, the signal peptide has a polynucleotide sequence of ATGGCACTGCCAGTGACCGCCCTGCTGCTGCCTCTGGCCCTGCTGCTGCACGCAGCA AGGCCA (SEQ ID NO: 3). In some embodiments, the signal peptide has a polynucleotide sequence with at least 80% sequence identity with SEQ ID NO: 3. In some embodiments, the signal peptide has a polynucleotide sequence with at least 90%, 95%, 97% or 99% sequence identity with SEQ ID NO: 3.

In some embodiments, the signal peptide has an amino acid sequence of METDTLLLWVLLLWVPGSTG (SEQ ID NO: 109). In some embodiments, the signal peptide has an amino acid sequence with at least 80% sequence identity with SEQ ID NO: 109. In some embodiments, the signal peptide has an amino acid sequence with at least 90%, 95%, 97% or 99% sequence identity with SEQ ID NO: 109.

### 5.1.1. Antigen Recognition Moiety

In some aspects, a chimeric antigen receptor (CAR) of the present disclosure comprises an antigen recognition moiety that recognizes and binds to a specific binding element on the target of interest. Non-limiting examples of the antigen recognition moiety include a single-chain variable fragment (scFv), nanobodies, ligands to cognate receptors, native receptors against targets, and small peptides.

Various antigens suitable for targeting with a CAR are known in the art, and each are suitable for use with the present disclosure. In some embodiments, the antigen recognition domain recognizes an antigen selected from the group consisting of HER2/neu, PSMA, Claudin18, CD20, CD5, BCMA, TAC1, GD2, mesothelin and CD19. In some embodiments, the antigen recognition domain recognizes HER2/neu. In some embodiments, the antigen recognition domain recognizes PSMA. In some embodiments, the antigen recognition domain recognizes Claudin 18. In some embodiments, the antigen recognition domain recognizes CD20. In some embodiments, the antigen recognition domain recognizes CD20. In some embodiments, the antigen recognition domain recognizes CD5. In some embodiments, the antigen recognition domain recognizes BCMA. In some embodiments, the antigen recognition domain recognizes CD19. In some embodiments, the antigen recognition domain recognizes mesothelin. In some embodiments, the antigen recognition domain recognizes TAC1. In some embodiments, the antigen recognition domain recognizes GD2. In certain aspects, the antigen recognition moiety is a single-chain variable fragment (scFv).

### 5.1.2. Hinge Domain

In certain aspects, provided herein is a chimeric antigen receptor (“CAR”) comprising a hinge domain. A hinge domain (also referred to as a spacer) is a structure between the antigen recognition moiety and the cell membrane. Non-limiting examples include, for example, a hinge domain derived from an IgG subclass (such as IgG1 and IgG4), IgD, CD28, CSF1R, a Fcγ receptor, and CD8 domains. In some embodiments, provided herein is a hinge domain lacking FcγR binding activity. In some embodiments, provided herein is a hinge domain derived from a native T cell molecule (e.g., CD28, or CD8). In some embodiments, a CAR provided herein comprises a hinge domain derived from CD8. In some embodiments, a CAR provided herein comprises a hinge domain derived from an IgG subclass. In specific embodiments, a CAR provided herein comprises a hinge domain derived from IgG1. In specific embodiments, a CAR provided herein comprises a hinge domain derived from IgG2. In specific embodiments, a CAR provided herein comprises a hinge domain derived from IgG3. In specific embodiments, a CAR provided herein comprises a hinge domain derived from IgG4. In some embodiments, a CAR provided herein comprises a hinge domain derived from IgD.

In specific embodiments, a CAR provided herein comprises a hinge domain comprising an amino acid sequence at least 90%, at least 95%, or 100% identical to an amino acid sequence provided in Table 1. In specific embodiments, a CAR provided herein comprises a hinge domain comprising an amino acid sequence at least 90%, at least 95%, or 100% identical to an amino acid sequence of SEQ ID NO:1 or SEQ ID NO:4. In specific embodiments, a CAR provided herein comprises a hinge domain comprising an amino acid sequence at least 90%, at least 95%, or 100% identical to an amino acid sequence of SEQ ID NO:1. In some embodiments, a CAR provided herein comprises a hinge domain comprising an amino acid sequence at least 90%, at least 95%, or 100% identical to SEQ ID NO: 1. In some embodiments, a CAR provided herein comprises a hinge domain comprising an amino acid sequence at least 90%, at least 95%, or 100% identical to SEQ ID NO: 4. In some embodiments, a CAR provided herein comprises a hinge domain comprising an amino acid sequence at least 90%, at least 95%, or 100% identical to SEQ ID NO: 5. In some embodiments, a CAR provided herein comprises a hinge domain comprising an amino acid sequence at least 90%, at least 95%, or 100% identical to SEQ ID NO: 6. In some embodiments, a CAR provided herein comprises a hinge domain comprising an amino acid sequence at least 90%, at least 95%, or 100% identical to SEQ ID NO: 7. In some embodiments, a CAR provided herein comprises a hinge domain comprising an amino acid sequence at least 90%, at least 95%, or 100% identical to SEQ ID NO: 8. In some embodiments, a CAR provided herein comprises a hinge domain comprising an amino acid sequence at least 90%, at least 95%, or 100% identical to SEQ ID NO: 9. In some embodiments, a CAR provided herein comprises a hinge domain comprising an amino acid sequence at least 90%, at least 95%, or 100% identical to SEQ ID NO: 10.

TABLE 1
Exemplary hinge domain sequences.

| Hinge Domain | Sequence | SEQ ID NO |
|---|---|---|
| CD8 | TTTPAPRPPTPAPTIASQPLSLRPEACRPAAGGAVHTRGLDFACD | 1 |
| CD8L | ALSNSIMYFSHFVPVFLPAKPTTTPAPRPPTPAPTIASQPLSLRPEA | 4 |
| | CRPAAGGAVHTRGLDFACD | |
| CD8S | ESKYGPPCPPCP | 5 |
| CD28 | LDNEKSNGTIIHVKGKHLCPSPLFPGPSKP | 6 |
| CD28 | KIEVMYPPPYLDNEKSNGTIIHVKGKHLCPSPLFPGPSKP | 7 |
| CD28 | IEVMYPPPYLDNEKSNGTIIHVKGKHLCPSPLFPGPSKP | 8 |
| FcγR1 | APVLNASVTSPLLEGNLVTLSCETKLLLQRPGLQLYFSFYMGSKTLR | 9 |
| | GRNTSSEYQILTARREDSGLYWCEAATEDGNVLKRSPELELQVLG | |
| CSF1R | PPEVSVIWTFINGSGTLLCAASGYPQPNVTWLQCSGHTDRCDEAQVL | 10 |
| | QVWDDPYPEVLSQEPFHKVTVQSLLTVETLEHNQTYECRAHNSVGSG | |
| | SWAFIPISAGAHTHPPDEFLFTP | |

### 5.1.3. Transmembrane Domain

In certain aspects, provided herein is a CAR comprising a transmembrane domain. A transmembrane domain is a structure that facilitates anchoring the CAR in a cell membrane, and generally consists of a hydrophobic α-helix that spans the cell membrane. Non-limiting examples include, for example, a hinge domain derived from an IgG subclass (such as IgG1 and IgG4), IgD, CD28, CSF1R, a Fcγ receptor, and CD8 domains. In some embodiments, the transmembrane domain is a single-span transmembrane (e.g., a transmembrane domain derived from CD4, CD8α, or CD28). In some embodiments, a CAR provided herein comprises a transmembrane domain derived from CD8. In some embodiments, a CAR provided herein does not comprise a transmembrane domain derived from CD8. In some embodiments, a CAR provided herein comprises a transmembrane domain derived from CD28. In some embodiments, a CAR provided herein does not comprise a transmembrane domain derived from CD28. In some embodiments, a CAR provided herein comprises a transmembrane domain derived from CD86. In some embodiments, a CAR provided herein does not comprise a transmembrane domain derived from CD86. In some embodiments, a CAR provided herein comprises a transmembrane domain derived from TLR4. In some embodiments, a CAR provided herein does not comprise a transmembrane domain derived from TLR4. In some embodiments, the transmembrane domain is derived from the same source as at least one of the intracellular domains. For example, the transmembrane domain can be derived from CD86 and at least one intracellular domain can also be derived from CD86. As a further example, the transmembrane can be derived from DECTIN-1 and at least one intracellular domain can also be derived from DECTIN-1.

As provided herein, the present disclosure relates in part to transmembrane domains that enhance macrophage activity. In some embodiments, the CAR provided herein comprises a transmembrane domain that enhances activity of a CAR in a macrophage, relative to the CAR having a transmembrane domain consisting of a CD8 transmembrane domain. In specific embodiments, the increase in activity comprises an increase in secretion of a pro-inflammatory cytokine, relative to the CAR having a transmembrane domain consisting of a CD8 transmembrane domain. In some embodiments, the pro-inflammatory cytokine is selected from the group consisting of CXCL10, CCL1, CCL2, CCL22, MIP-1α/CCL3, IL-1β, IFNγ, IL-10, IL-6, TNFA, and CCL20, or a combination thereof. In specific embodiments, the pro-inflammatory cytokine comprises MIP-1α/CCL3. Techniques known to one of skill in the art or described herein (e.g., Section 5.9.1 or in the Examples) may be used to assess secretion of the pro-inflammatory cytokine. In specific embodiments, secretion of the pro-inflammatory cytokine is measured by ELISA after culturing the macrophage for about 24 hours in the presence of a target antigen.

In specific embodiments, the increase in secretion of the pro-inflammatory cytokine is about 5-fold to about 100-fold, about 5-fold to about 50-fold, about 5-fold to about 40-fold, about 5-fold to about 30-fold, about 5-fold to about 20-fold, about 10-fold to about 100-fold, about 10-fold to about 50-fold, about 10-fold to about 40-fold, about 10-fold to about 30-fold, about 10-fold to about 20-fold, 20-fold to about 100-fold, about 20-fold to about 50-fold, about 20-fold to about 40-fold, or about 20-fold to about 30-fold, relative to an otherwise identical CAR having an transmembrane domain consisting of a CD8 transmembrane domain. In some embodiments, the increase in secretion of the pro-inflammatory cytokine is about 5-fold, relative to an otherwise identical CAR having an transmembrane domain consisting of a CD8 transmembrane domain. In some embodiments, the increase in secretion of the pro-inflammatory cytokine is about 10-fold, relative to an otherwise identical CAR having an transmembrane domain consisting of a CD8 transmembrane domain. In some embodiments, the increase in secretion of the pro-inflammatory cytokine is about 20-fold, relative to an otherwise identical CAR having an transmembrane domain consisting of a CD8 transmembrane domain. In some embodiments, the increase in secretion of the pro-inflammatory cytokine is about 30-fold, relative to an otherwise identical CAR having an transmembrane domain consisting of a CD8 transmembrane domain. In some embodiments, the increase in secretion of the pro-inflammatory cytokine is about 40-fold, relative to an otherwise identical CAR having an transmembrane domain consisting of a CD8 transmembrane domain. In some embodiments, the increase in secretion of the pro-inflammatory cytokine is about 50-fold, relative to an otherwise identical CAR having an transmembrane domain consisting of a CD8 transmembrane domain. In some embodiments, the increase in secretion of the pro-inflammatory cytokine is about 75-fold, relative to an otherwise identical CAR having an transmembrane domain consisting of a CD8 transmembrane domain. In some embodiments, the increase in secretion of the pro-inflammatory cytokine is about 100-fold, relative to an otherwise identical CAR having an transmembrane domain consisting of a CD8 transmembrane domain. In some embodiments, the increase in secretion of the pro-inflammatory cytokine is more than about 100-fold, relative to an otherwise identical CAR having an transmembrane domain consisting of a CD8 transmembrane domain.

In certain embodiments, the increase in secretion of the pro-inflammatory cytokine comprises a composite score of the secretion of two of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. For example, in some embodiments, the secretion amount of two or more pro-inflammatory cytokines can be determined using techniques known in the art or provided herein, and an average score of the two or more cytokines can be calculated. In some embodiments, the secretion of the two or more pro-inflammatory cytokines can be normalized against an otherwise identical CAR having a transmembrane domain consisting of a CD8 transmembrane domain, and a composite score can be generated by calculating the sum of the two or more proinflammatory cytokines.

In certain embodiments, the increase in secretion of the pro-inflammatory cytokine comprises a composite score of the secretion of three of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of three or more pro-inflammatory cytokines can be determined using techniques known in the art or provided herein, and an average score of the three or more cytokines can be calculated. In some embodiments, the secretion of the three or more pro-inflammatory cytokines can be normalized against an otherwise identical CAR having a transmembrane domain consisting of a CD8 transmembrane domain, and a composite score can be generated by calculating the sum of the three or more pro-inflammatory cytokines.

In certain embodiments, the increase in secretion of the pro-inflammatory cytokine comprises a composite score of the secretion of four of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of four or more pro-inflammatory cytokines can be determined using techniques known in the art or provided herein, and an average score of the four or more cytokines can be calculated. In some embodiments, the secretion of the four or more pro-inflammatory cytokines can be normalized against an otherwise identical CAR having a transmembrane domain consisting of a CD8 transmembrane domain, and a composite score can be generated by calculating the sum of the four or more pro-inflammatory cytokines.

In certain embodiments, the increase in secretion of the pro-inflammatory cytokine comprises a composite score of the secretion of five of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of five or more pro-inflammatory cytokines can be determined using techniques known in the art or provided herein, and an average score of the five or more cytokines can be calculated. In some embodiments, the secretion of the five or more pro-inflammatory cytokines can be normalized against an otherwise identical CAR having a transmembrane domain consisting of a CD8 transmembrane domain, and a composite score can be generated by calculating the sum of the five or more pro-inflammatory cytokines.

In certain embodiments, the increase in secretion of the pro-inflammatory cytokine comprises a composite score of the secretion of six of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of six or more pro-inflammatory cytokines can be determined using techniques known in the art or provided herein, and an average score of the six or more cytokines can be calculated. In some embodiments, the secretion of the six or more pro-inflammatory cytokines can be normalized against an otherwise identical CAR having a transmembrane domain consisting of a CD8 transmembrane domain, and a composite score can be generated by calculating the sum of the six or more pro-inflammatory cytokines.

In certain embodiments, the increase in secretion of the pro-inflammatory cytokine comprises a composite score of the secretion of seven of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of seven or more pro-inflammatory cytokines can be determined using techniques known in the art or provided herein, and an average score of the seven or more cytokines can be calculated. In some embodiments, the secretion of the seven or more pro-inflammatory cytokines can be normalized against an otherwise identical CAR having a transmembrane domain consisting of a CD8 transmembrane domain, and a composite score can be generated by calculating the sum of the seven or more pro-inflammatory cytokines.

In certain embodiments, the increase in secretion of the pro-inflammatory cytokine comprises a composite score of the secretion of eight of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of eight or more pro-inflammatory cytokines can be determined using techniques known in the art or provided herein, and an average score of the eight or more cytokines can be calculated. In some embodiments, the secretion of the eight or more pro-inflammatory cytokines can be normalized against an otherwise identical CAR having a transmembrane domain consisting of a CD8 transmembrane domain, and a composite score can be generated by calculating the sum of the eight or more pro-inflammatory cytokines.

In certain embodiments, the increase in secretion of the pro-inflammatory cytokine comprises a composite score of the secretion of nine of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of nine or more pro-inflammatory cytokines can be determined using techniques known in the art or provided herein, and an average score of the nine or more cytokines can be calculated. In some embodiments, the secretion of the nine or more pro-inflammatory cytokines can be normalized against an otherwise identical CAR having a transmembrane domain consisting of a CD8 transmembrane domain, and a composite score can be generated by calculating the sum of the nine or more pro-inflammatory cytokines.

In certain embodiments, the increase in secretion of the pro-inflammatory cytokine comprises a composite score of the secretion of ten of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of ten or more pro-inflammatory cytokines can be determined using techniques known in the art or provided herein, and an average score of the ten or more cytokines can be calculated. In some embodiments, the secretion of the ten or more pro-inflammatory cytokines can be normalized against an otherwise identical CAR having a transmembrane domain consisting of a CD8 transmembrane domain, and a composite score can be generated by calculating the sum of the ten or more pro-inflammatory cytokines.

In certain embodiments, the increase in secretion of the pro-inflammatory cytokine comprises a composite score of the secretion of eleven of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of eleven or more pro-inflammatory cytokines can be determined using techniques known in the art or provided herein, and an average score of the eleven or more cytokines can be calculated. In some embodiments, the secretion of the eleven or more pro-inflammatory cytokines can be normalized against an otherwise identical CAR having a transmembrane domain consisting of a CD8 transmembrane domain, and a composite score can be generated by calculating the sum of the eleven or more pro-inflammatory cytokines.

In some embodiments, the increase in activity comprises an increase in macrophage phagocytosis. Techniques known to one of skill in the art or described herein (e.g., Section 5.9.2 and in the Example) may be used to assess phagocytosis. For example, in some embodiments phagocytosis is measured by flow cytometry after about four hours of co-incubation of a macrophage expressing the CAR and a target cell expressing a target antigen. In some embodiments, the increase in phagocytosis is about 5% to about 50%, about 5% to about 40%, about 5% to about 30%, about 5% to about 20%, about 5% to about 10%, about 10% to about 50%, about 10% to about 40%, about 10% to about 30%, about 10% to about 20%, about 20% to about 50%, about 20% to about 40%, about 20% to about 30%, about 30% to about 50%, about 30% to about 40%, or about 40% to about 50%, relative to an otherwise identical CAR having an transmembrane domain consisting of a CD8 transmembrane domain. In some embodiments, the increase in phagocytosis is about 5%, relative to an otherwise identical CAR having an transmembrane domain consisting of a CD8 transmembrane domain. In some embodiments, the increase in phagocytosis is about 10%, relative to an otherwise identical CAR having an transmembrane domain consisting of a CD8 transmembrane domain. In some embodiments, the increase in phagocytosis is about 20%, relative to an otherwise identical CAR having an transmembrane domain consisting of a CD8 transmembrane domain. In some embodiments, the increase in phagocytosis is about 30%, relative to an otherwise identical CAR having an transmembrane domain consisting of a CD8 transmembrane domain. In some embodiments, the increase in phagocytosis is about 40%, relative to an otherwise identical CAR having an transmembrane domain consisting of a CD8 transmembrane domain. In some embodiments, the increase in phagocytosis is about 50%, relative to an otherwise identical CAR having an transmembrane domain consisting of a CD8 transmembrane domain. In some embodiments, the increase in phagocytosis is more than about 50%, relative to an otherwise identical CAR having an transmembrane domain consisting of a CD8 transmembrane domain.

In some embodiments, the increase in activity of the macrophage expressing a CAR provided herein is an increase in secretion of a pro-inflammatory cytokine and an increase in phagocytosis, relative to an otherwise identical CAR having an transmembrane domain consisting of a CD8 transmembrane domain. In some embodiments, both secretion of the pro-inflammatory cytokine and phagocytosis are increased by about 5% to about 50%, about 5% to about 40%, about 5% to about 30%, about 5% to about 20%, about 5% to about 10%, about 10% to about 50%, about 10% to about 40%, about 10% to about 30%, about 10% to about 20%, about 20% to about 50%, about 20% to about 40%, about 20% to about 30%, about 30% to about 50%, about 30% to about 40%, or about 40% to about 50%, relative to an otherwise identical CAR having an transmembrane domain consisting of a CD8 transmembrane domain. In some embodiments, both secretion of the pro-inflammatory cytokine and phagocytosis are increased by at least 5%, relative to an otherwise identical CAR having an transmembrane domain consisting of a CD8 transmembrane domain. In some embodiments, both secretion of the pro-inflammatory cytokine and phagocytosis are increased by at least 10%, relative to an otherwise identical CAR having an transmembrane domain consisting of a CD8 transmembrane domain. In some embodiments, both secretion of the pro-inflammatory cytokine and phagocytosis are increased by at least 20%, relative to an otherwise identical CAR having an transmembrane domain consisting of a CD8 transmembrane domain. In some embodiments, both secretion of the pro-inflammatory cytokine and phagocytosis are increased by at least 30%, relative to an otherwise identical CAR having an transmembrane domain consisting of a CD8 transmembrane domain. In some embodiments, both secretion of the pro-inflammatory cytokine and phagocytosis are increased by at least 40%, relative to an otherwise identical CAR having an transmembrane domain consisting of a CD8 transmembrane domain. In some embodiments, both secretion of the pro-inflammatory cytokine and phagocytosis are increased by at least 50%, relative to an otherwise identical CAR having an transmembrane domain consisting of a CD8 transmembrane domain. In some embodiments, both secretion of the pro-inflammatory cytokine and phagocytosis are increased by more than about 50%, relative to an otherwise identical CAR having an transmembrane domain consisting of a CD8 transmembrane domain.

In some embodiments, the increase in activity of the macrophage expressing a CAR provided herein is an increase in secretion of a pro-inflammatory cytokine and no increase in phagocytosis, relative to an otherwise identical CAR having an transmembrane domain consisting of a CD8 transmembrane domain. In some embodiments, secretion of the pro-inflammatory cytokine is increased by about 5% to about 50%, about 5% to about 40%, about 5% to about 30%, about 5% to about 20%, about 5% to about 10%, about 10% to about 50%, about 10% to about 40%, about 10% to about 30%, about 10% to about 20%, about 20% to about 50%, about 20% to about 40%, about 20% to about 30%, about 30% to about 50%, about 30% to about 40%, or about 40% to about 50%, and there is no increase in phagocytosis, relative to an otherwise identical CAR having an transmembrane domain consisting of a CD8 transmembrane domain. In some embodiments, secretion of the pro-inflammatory cytokine and phagocytosis is increased by at least 5%, and there is no increase in phagocytosis, relative to an otherwise identical CAR having an transmembrane domain consisting of a CD8 transmembrane domain. In some embodiments, secretion of the pro-inflammatory cytokine and phagocytosis is increased by at least 10%, and there is no increase in phagocytosis, relative to an otherwise identical CAR having an transmembrane domain consisting of a CD8 transmembrane domain. In some embodiments, secretion of the pro-inflammatory cytokine and phagocytosis is increased by at least 20%, and there is no increase in phagocytosis, relative to an otherwise identical CAR having an transmembrane domain consisting of a CD8 transmembrane domain. In some embodiments, secretion of the pro-inflammatory cytokine and phagocytosis is increased by at least 30%, and there is no increase in phagocytosis, relative to an otherwise identical CAR having an transmembrane domain consisting of a CD8 transmembrane domain. In some embodiments, secretion of the pro-inflammatory cytokine and phagocytosis is increased by at least 40%, and there is no increase in phagocytosis, relative to an otherwise identical CAR having an transmembrane domain consisting of a CD8 transmembrane domain. In some embodiments, secretion of the pro-inflammatory cytokine and phagocytosis is increased by at least 50%, and there is no increase in phagocytosis, relative to an otherwise identical CAR having an transmembrane domain consisting of a CD8 transmembrane domain. In some embodiments, secretion of the pro-inflammatory cytokine and phagocytosis is increased by more than about 50%, and there is no increase in phagocytosis, relative to an otherwise identical CAR having an transmembrane domain consisting of a CD8 transmembrane domain.

Exemplary transmembrane domains are provided in Table 2. In some embodiments, a CAR provided herein comprises a transmembrane domain selected from the group consisting of a IL1R1 transmembrane domain, a CD8 transmembrane domain, a CD28 transmembrane domain, a DAP12 transmembrane domain, a CD11b transmembrane domain, a VEGFR2 transmembrane domain, a IFNγR1 transmembrane domain, a CSF3R transmembrane domain, a CSF2R transmembrane domain, a SLAMF7 transmembrane domain, a DECTIN-1 transmembrane domain, a DECTIN-3 transmembrane domain, a TLR4 transmembrane domain, a CSF1R transmembrane domain, a CD86 transmembrane domain, a macrophage-inducible C-type lectin (MINCLE) transmembrane domain, a BDCA2 transmembrane domain, a MDL1 transmembrane domain, and a CD40 transmembrane domain. In some embodiments, a CAR provided herein comprises a transmembrane domain selected from the group consisting of a CD8 transmembrane domain, a SLAMF7 transmembrane domain, a CD86 transmembrane domain, a DECTIN-1 transmembrane domain, and a CSF1R transmembrane domain. In specific embodiments, a CAR provided herein comprises a transmembrane domain comprising a CD8 transmembrane domain. In specific embodiments, a CAR provided herein comprises a transmembrane domain comprising a SLAMF7 transmembrane domain. In specific embodiments, a CAR provided herein comprises a transmembrane domain comprising a CD86 transmembrane domain. In specific embodiments, a CAR provided herein comprises a transmembrane domain comprising a DECTIN-1 transmembrane domain. In specific embodiments, a CAR provided herein comprises a transmembrane domain comprising a CD8 transmembrane domain. In specific embodiments, a CAR provided herein comprises a transmembrane domain comprising a IL-1R1 transmembrane domain. In specific embodiments, a CAR provided herein comprises a transmembrane domain comprising a VEGFR2 transmembrane domain. In specific embodiments, a CAR provided herein comprises a transmembrane domain comprising a IFNγR1 transmembrane domain. In specific embodiments, a CAR provided herein comprises a transmembrane domain comprising a CSF3R transmembrane domain. In specific embodiments, a CAR provided herein comprises a transmembrane domain comprising a CSF2R transmembrane domain. In specific embodiments, a CAR provided herein comprises a transmembrane domain comprising a CD86 transmembrane domain. In specific embodiments, a CAR provided herein comprises a transmembrane domain comprising a TLR4 transmembrane domain. In specific embodiments, a CAR provided herein comprises a transmembrane domain comprising a CD40 transmembrane domain. In specific embodiments, a CAR provided herein comprises a transmembrane domain comprising a CSF1R transmembrane domain. In specific embodiments, a CAR provided herein comprises a transmembrane domain comprising a DECTIN-3 transmembrane domain. In some embodiments, a CAR provided herein does not comprise a transmembrane domain derived from CD8. In some embodiments, a CAR provided herein does not comprise a transmembrane domain derived from CD28. In some embodiments, a CAR provided herein does not comprise a transmembrane domain derived from TLR4. In some embodiments, a CAR provided herein does not comprise a transmembrane domain derived from CD86.

In specific embodiments, a CAR provided herein comprises a transmembrane domain comprising an amino acid sequence at least 90%, at least 95%, or 100% identical to an amino acid sequence provided in Table 2. In specific embodiments, a CAR provided herein comprises a transmembrane domain comprising an amino acid sequence at least 90%, at least 95%, or 100% identical to SEQ ID NO:11, SEQ ID NO:12, SEQ ID NO:13, or SEQ ID NO:14. In some embodiments, a CAR provided herein comprises a transmembrane domain comprising an amino acid sequence at least 90%, at least 95%, or 100% identical to SEQ ID NO: 11. In some embodiments, a CAR provided herein comprises a transmembrane domain comprising an amino acid sequence at least 90%, at least 95%, or 100% identical to SEQ ID NO: 12. In some embodiments, a CAR provided herein comprises a transmembrane domain comprising an amino acid sequence at least 90%, at least 95%, or 100% identical to SEQ ID NO: 13. In some embodiments, a CAR provided herein comprises a transmembrane domain comprising an amino acid sequence at least 90%, at least 95%, or 100% identical to SEQ ID NO: 14. In some embodiments, a CAR provided herein comprises a transmembrane domain comprising an amino acid sequence at least 90%, at least 95%, or 100% identical to SEQ ID NO: 15. In some embodiments, a CAR provided herein comprises a transmembrane domain comprising an amino acid sequence at least 90%, at least 95%, or 100% identical to SEQ ID NO: 16. In some embodiments, a CAR provided herein comprises a transmembrane domain comprising an amino acid sequence at least 90%, at least 95%, or 100% identical to SEQ ID NO: 17. In some embodiments, a CAR provided herein comprises a transmembrane domain comprising an amino acid sequence at least 90%, at least 95%, or 100% identical to SEQ ID NO: 18. In some embodiments, a CAR provided herein comprises a transmembrane domain comprising an amino acid sequence at least 90%, at least 95%, or 100% identical to SEQ ID NO: 19. In some embodiments, a CAR provided herein comprises a transmembrane domain comprising an amino acid sequence at least 90%, at least 95%, or 100% identical to SEQ ID NO: 20. In some embodiments, a CAR provided herein comprises a transmembrane domain comprising an amino acid sequence at least 90%, at least 95%, or 100% identical to SEQ ID NO: 21. In some embodiments, a CAR provided herein comprises a transmembrane domain comprising an amino acid sequence at least 90%, at least 95%, or 100% identical to SEQ ID NO: 22. In some embodiments, a CAR provided herein comprises a transmembrane domain comprising an amino acid sequence at least 90%, at least 95%, or 100% identical to SEQ ID NO: 23. In some embodiments, a CAR provided herein comprises a transmembrane domain comprising an amino acid sequence at least 90%, at least 95%, or 100% identical to SEQ ID NO: 24. In some embodiments, a CAR provided herein comprises a transmembrane domain comprising an amino acid sequence at least 90%, at least 95%, or 100% identical to SEQ ID NO: 25. In some embodiments, a CAR provided herein comprises a transmembrane domain comprising an amino acid sequence at least 90%, at least 95%, or 100% identical to SEQ ID NO: 26. In some embodiments, a CAR provided herein comprises a transmembrane domain comprising an amino acid sequence at least 90%, at least 95%, or 100% identical to SEQ ID NO: 27. In some embodiments, a CAR provided herein comprises a transmembrane domain comprising an amino acid sequence at least 90%, at least 95%, or 100% identical to SEQ ID NO: 28. In some embodiments, a CAR provided herein comprises a transmembrane domain comprising an amino acid sequence at least 90%, at least 95%, or 100% identical to SEQ ID NO: 29. In some embodiments, a CAR provided herein comprises a transmembrane domain comprising an amino acid sequence at least 90%, at least 95%, or 100% identical to SEQ ID NO: 30. In some embodiments, a CAR provided herein comprises a transmembrane domain comprising an amino acid sequence at least 90%, at least 95%, or 100% identical to SEQ ID NO: 31. In some embodiments, a CAR provided herein comprises a transmembrane domain comprising an amino acid sequence at least 90%, at least 95%, or 100% identical to SEQ ID NO: 32. In some embodiments, a CAR provided herein comprises a transmembrane domain comprising an amino acid sequence at least 90%, at least 95%, or 100% identical to SEQ ID NO: 33. In some embodiments, a CAR provided herein comprises a transmembrane domain comprising an amino acid sequence at least 90%, at least 95%, or 100% identical to SEQ ID NO: 34. In some embodiments, a CAR provided herein comprises a transmembrane domain comprising an amino acid sequence at least 90%, at least 95%, or 100% identical to SEQ ID NO: 35. In some embodiments, a CAR provided herein comprises a transmembrane domain comprising an amino acid sequence at least 90%, at least 95%, or 100% identical to SEQ ID NO: 36. In some embodiments, a CAR provided herein comprises a transmembrane domain comprising an amino acid sequence at least 90%, at least 95%, or 100% identical to SEQ ID NO: 37. In some embodiments, a CAR provided herein comprises a transmembrane domain comprising an amino acid sequence at least 90%, at least 95%, or 100% identical to SEQ ID NO: 38. In some embodiments, a CAR provided herein comprises a transmembrane domain comprising an amino acid sequence at least 90%, at least 95%, or 100% identical to SEQ ID NO: 39. In some embodiments, a CAR provided herein comprises a transmembrane domain comprising an amino acid sequence at least 90%, at least 95%, or 100% identical to SEQ ID NO: 17 or SEQ ID NO: 18. In some embodiments, a CAR provided herein comprises a transmembrane domain comprising an amino acid sequence at least 90%, at least 95%, or 100% identical to SEQ ID NO: 26 or SEQ ID NO:27.

TABLE 2
Exemplary transmembrane domains

| Transmembrane | | SEQ |
|---|---|---|
| Domain | Sequence | ID NO |
| CD8 | IYIWAPLAGTCGVLLLSLVIT | 11 |
| SLAMF7 | VLLCLLLVPLLLSLFVLGLFL | 12 |
| CD86 | WITAVLPTVIICVMVFCLILW | 13 |
| DECTIN-1 CLEC7A | GLVVAIVLIVLCLIGLIVAIL | 14 |
| DECTIN-2/CLEC6A | VVCSVIFCASLLAISIGAVSW | 15 |
| DECTIN-3/CLEC4D | SAIFCVSLLLIFVVAIVSPIL | 16 |
| CD40 | ALVVIPIIFGILFAILLVLVFI | 17 |
| CD40 | AVILFFFTFFITTMVMLAAL | 18 |
| TLR1 | LLIVTIVATMLVLAVTVTSLC | 19 |
| TLR2 | ALVSGMCCALFLLILLTGVL | 20 |
| TLR3 | FFMINTSILLIFIFIVLLIH | 21 |
| TLR4 | TIIGVSVLSVLVVSVVAVLVY | 22 |
| TLR5 | FSLFIVCTVTLTLFLMTILTV | 23 |
| TLR6 | LIVTIGATMLVLAVTVTSLCI | 24 |
| TLR7 | LILFSLSISVSLFLMVMMTAS | 25 |
| CD28 | FWVLVVVGGVLACYSLLVTVAFIIFWV | 26 |
| CD28 | RSKRSRLLHSDYMNMTPRRPGPTRKHYQPYAPPRDFAAYRS | 27 |
| MDL1 | FYLLFLTMGVVKLVVVILGSIIM | 28 |
| DAP12 | GVLAGIVMGDLVLTVLIALAV | 29 |
| IL1R1 | HMIGICVTLTVIIVCSVFIY | 30 |
| BDCA2 | HPVVSSVTFCVSLLLISVVAMSW | 31 |
| VEGFR2 | IIILVGTAVIAMFFWLLLVII | 32 |
| CSFIR | VVVACMSIMALLLLLLLLLLY | 33 |
| CSF2R | NLGSVYIYVLLIVGTLVCGIVLGFLF | 34 |
| CSF3R | IILGLFGLLLLLTCLCGTAWLCC | 35 |
| CD11b | PLPLIVGSSVGGLLLLALITAALY | 36 |
| IFNγR1 | SLWIPVVAALLLFLVLSLVFI | 37 |
| NKG2D | SNLFVASWIAVMIIFRIGMAVAIFCCFFFP | 38 |
| MINCLE/CLEC4E | TIFCASLFLIPIGAVTWLFMQ | 39 |

### 5.1.4. Intracellular Domain.

In certain aspects, provided herein is a CAR comprising one or more intracellular domains. An intracellular domain transmits activation signals. In specific embodiments, an intracellular domain provided herein comprises a non-lymphoid intracellular signaling domain (such as a non-lymphoid intracellular signaling domain described in Section 5.1.4.1) and an intracellular domain comprising one or more immune-receptor-tyrosine-based-activation-motif (ITAM) (such as an ITAM containing intracellular signaling domain described in Section 5.1.4.2).

As provided herein, the present disclosure relates, in part, to intracellular domains that enhance macrophage activity. In some embodiments, a CAR provided herein comprises at least one intracellular domain that enhances activity of a CAR in a macrophage, relative to an otherwise identical CAR having an intracellular domain consisting of a CD3ζ intracellular signaling domain. In specific embodiments, the increase in activity comprises an increase in secretion of a pro-inflammatory cytokine, relative to an otherwise identical CAR having an intracellular domain consisting of a CD3ζ intracellular signaling domain. In certain embodiments, the increase in activity comprises an increase in phagocytosis. In some embodiments, the increase in activity comprises an increase in secretion of a pro-inflammatory cytokine and an increase in phagocytosis. In some embodiments, the increase in activity comprises M1 polarization of the macrophage. In some embodiments, the pro-inflammatory cytokine is selected from the group consisting of CXCL10, CCL1, CCL2, CCL22, MIP-1α/CCL3, IL-1β, IFNγ, IL-10, IL-6, TNFA, and CCL20, or a combination thereof. In specific embodiments, the pro-inflammatory cytokine comprises MIP-1α/CCL3. Techniques known to one of skill in the art or described herein (e.g., Section 5.9.1 or in the Examples) may be used to assess secretion of the pro-inflammatory cytokine. In specific embodiments, secretion of the pro-inflammatory cytokine is measured by ELISA after culturing the macrophage for about 24 hours in the presence of a target antigen.

In specific embodiments, the increase in secretion of the pro-inflammatory cytokine is about 5-fold to about 100-fold, about 5-fold to about 50-fold, about 5-fold to about 40-fold, about 5-fold to about 30-fold, about 5-fold to about 20-fold, about 10-fold to about 100-fold, about 10-fold to about 50-fold, about 10-fold to about 40-fold, about 10-fold to about 30-fold, about 10-fold to about 20-fold, 20-fold to about 100-fold, about 20-fold to about 50-fold, about 20-fold to about 40-fold, or about 20-fold to about 30-fold, relative to an otherwise identical CAR having an intracellular domain consisting of a CD3ζ intracellular signaling domain. In some embodiments, the increase in secretion of the pro-inflammatory cytokine is about 5-fold, relative to an otherwise identical CAR having an intracellular domain consisting of a CD3ζ intracellular signaling domain. In some embodiments, the increase in secretion of the pro-inflammatory cytokine is about 10-fold, relative to an otherwise identical CAR having an intracellular domain consisting of a CD3ζ intracellular signaling domain. In some embodiments, the increase in secretion of the pro-inflammatory cytokine is about 20-fold, relative to an otherwise identical CAR having an intracellular domain consisting of a CD3ζ intracellular signaling domain. In some embodiments, the increase in secretion of the pro-inflammatory cytokine is about 30-fold, relative to an otherwise identical CAR having an intracellular domain consisting of a CD3ζ intracellular signaling domain. In some embodiments, the increase in secretion of the pro-inflammatory cytokine is about 40-fold, relative to an otherwise identical CAR having an intracellular domain consisting of a CD3ζ intracellular signaling domain. In some embodiments, the increase in secretion of the pro-inflammatory cytokine is about 50-fold, relative to an otherwise identical CAR having an intracellular domain consisting of a CD3 intracellular signaling domain. In some embodiments, the increase in secretion of the pro-inflammatory cytokine is about 75-fold, relative to an otherwise identical CAR having an intracellular domain consisting of a CD3ζ intracellular signaling domain. In some embodiments, the increase in secretion of the pro-inflammatory cytokine is about 100-fold, relative to an otherwise identical CAR having an intracellular domain consisting of a CD3ζ intracellular signaling domain. In some embodiments, the increase in secretion of the pro-inflammatory cytokine is more than about 100-fold, relative to an otherwise identical CAR having an intracellular domain consisting of a CD3ζ intracellular signaling domain.

In certain embodiments, the increase in secretion of the pro-inflammatory cytokine comprises a composite score of the secretion of two of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. For example, in some embodiments, the secretion amount of two or more pro-inflammatory cytokines can be determined using techniques known in the art or provided herein, and an average score of the two or more cytokines can be calculated. In some embodiments, the secretion of the two or more pro-inflammatory cytokines can be normalized against an otherwise identical CAR having an intracellular domain consisting of a CD3ζ intracellular signaling domain, and a composite score can be generated by calculating the sum of the two or more proinflammatory cytokines.

In certain embodiments, the increase in secretion of the pro-inflammatory cytokine comprises a composite score of the secretion of three of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of three or more pro-inflammatory cytokines can be determined using techniques known in the art or provided herein, and an average score of the three or more cytokines can be calculated. In some embodiments, the secretion of the three or more pro-inflammatory cytokines can be normalized against an otherwise identical CAR having an intracellular domain consisting of a CD3ζ intracellular signaling domain, and a composite score can be generated by calculating the sum of the three or more pro-inflammatory cytokines.

In certain embodiments, the increase in secretion of the pro-inflammatory cytokine comprises a composite score of the secretion of four of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of four or more pro-inflammatory cytokines can be determined using techniques known in the art or provided herein, and an average score of the four or more cytokines can be calculated. In some embodiments, the secretion of the four or more pro-inflammatory cytokines can be normalized against an otherwise identical CAR having an intracellular domain consisting of a CD3ζ intracellular signaling domain, and a composite score can be generated by calculating the sum of the four or more pro-inflammatory cytokines.

In certain embodiments, the increase in secretion of the pro-inflammatory cytokine comprises a composite score of the secretion of five of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of five or more pro-inflammatory cytokines can be determined using techniques known in the art or provided herein, and an average score of the five or more cytokines can be calculated. In some embodiments, the secretion of the five or more pro-inflammatory cytokines can be normalized against an otherwise identical CAR having an intracellular domain consisting of a CD3ζ intracellular signaling domain, and a composite score can be generated by calculating the sum of the five or more pro-inflammatory cytokines.

In certain embodiments, the increase in secretion of the pro-inflammatory cytokine comprises a composite score of the secretion of six of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of six or more pro-inflammatory cytokines can be determined using techniques known in the art or provided herein, and an average score of the six or more cytokines can be calculated. In some embodiments, the secretion of the six or more pro-inflammatory cytokines can be normalized against an otherwise identical CAR having an intracellular domain consisting of a CD3ζ intracellular signaling domain, and a composite score can be generated by calculating the sum of the six or more pro-inflammatory cytokines.

In certain embodiments, the increase in secretion of the pro-inflammatory cytokine comprises a composite score of the secretion of seven of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of seven or more pro-inflammatory cytokines can be determined using techniques known in the art or provided herein, and an average score of the seven or more cytokines can be calculated. In some embodiments, the secretion of the seven or more pro-inflammatory cytokines can be normalized against an otherwise identical CAR having an intracellular domain consisting of a CD3ζ intracellular signaling domain, and a composite score can be generated by calculating the sum of the seven or more pro-inflammatory cytokines.

In certain embodiments, the increase in secretion of the pro-inflammatory cytokine comprises a composite score of the secretion of eight of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of eight or more pro-inflammatory cytokines can be determined using techniques known in the art or provided herein, and an average score of the eight or more cytokines can be calculated. In some embodiments, the secretion of the eight or more pro-inflammatory cytokines can be normalized against an otherwise identical CAR having an intracellular domain consisting of a CD3ζ intracellular signaling domain, and a composite score can be generated by calculating the sum of the eight or more pro-inflammatory cytokines.

In certain embodiments, the increase in secretion of the pro-inflammatory cytokine comprises a composite score of the secretion of nine of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of nine or more pro-inflammatory cytokines can be determined using techniques known in the art or provided herein, and an average score of the nine or more cytokines can be calculated. In some embodiments, the secretion of the nine or more pro-inflammatory cytokines can be normalized against an otherwise identical CAR having an intracellular domain consisting of a CD3ζ intracellular signaling domain, and a composite score can be generated by calculating the sum of the nine or more pro-inflammatory cytokines.

In certain embodiments, the increase in secretion of the pro-inflammatory cytokine comprises a composite score of the secretion of ten of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of ten or more pro-inflammatory cytokines can be determined using techniques known in the art or provided herein, and an average score of the ten or more cytokines can be calculated. In some embodiments, the secretion of the ten or more pro-inflammatory cytokines can be normalized against an otherwise identical CAR having an intracellular domain consisting of a CD3ζ intracellular signaling domain, and a composite score can be generated by calculating the sum of the ten or more pro-inflammatory cytokines.

In certain embodiments, the increase in secretion of the pro-inflammatory cytokine comprises a composite score of the secretion of eleven of more pro-inflammatory cytokines, such as a pro-inflammatory cytokine selected from the group consisting of CXCL10, CCL1, CCL2, CCL22, MIP-1α/CCL3, IL-1β, IFNγ, IL-10, IL-6, TNFA, and CCL20. In some embodiments, the secretion amount of eleven or more pro-inflammatory cytokines can be determined using techniques known in the art or provided herein, and an average score of the eleven or more cytokines can be calculated. In some embodiments, the secretion of the eleven or more pro-inflammatory cytokines can be normalized against an otherwise identical CAR having an intracellular domain consisting of a CD3ζ intracellular signaling domain, and a composite score can be generated by calculating the sum of the eleven or more pro-inflammatory cytokines.

In some embodiments, the increase in activity comprises an increase in macrophage phagocytosis. Techniques known to one of skill in the art or described herein (e.g., Section 5.9.2 or in the Examples) may be used to assess phagocytosis. For example, in some embodiments phagocytosis is measured by flow cytometry after about four hours of co-incubation of a macrophage expressing the CAR and a target cell expressing a target antigen. In some embodiments, the increase in phagocytosis is about 5% to about 50%, about 5% to about 40%, about 5% to about 30%, about 5% to about 20%, about 5% to about 10%, about 10% to about 50%, about 10% to about 40%, about 10% to about 30%, about 10% to about 20%, about 20% to about 50%, about 20% to about 40%, about 20% to about 30%, about 30% to about 50%, about 30% to about 40%, or about 40% to about 50%, relative to an otherwise identical CAR having an intracellular domain consisting of a CD3ζ intracellular signaling domain. In some embodiments, the increase in phagocytosis is about 5%, relative to an otherwise identical CAR having an intracellular domain consisting of a CD3ζ intracellular signaling domain. In some embodiments, the increase in phagocytosis is about 10%, relative to an otherwise identical CAR having an intracellular domain consisting of a CD3ζ intracellular signaling domain. In some embodiments, the increase in phagocytosis is about 20%, relative to an otherwise identical CAR having an intracellular domain consisting of a CD3ζ intracellular signaling domain. In some embodiments, the increase in phagocytosis is about 30%, relative to an otherwise identical CAR having an intracellular domain consisting of a CD3ζ intracellular signaling domain. In some embodiments, the increase in phagocytosis is about 40%, relative to an otherwise identical CAR having an intracellular domain consisting of a CD3ζ intracellular signaling domain. In some embodiments, the increase in phagocytosis is about 50%, relative to an otherwise identical CAR having an intracellular domain consisting of a CD3ζ intracellular signaling domain. In some embodiments, the increase in phagocytosis is more than about 50%, relative to an otherwise identical CAR having an intracellular domain consisting of a CD3ζ intracellular signaling domain.

In some embodiments, the increase in activity of the macrophage expressing a CAR provided herein is an increase in secretion of a pro-inflammatory cytokine and an increase in phagocytosis, relative to an otherwise identical CAR having an intracellular domain consisting of a CD3ζ intracellular signaling domain. In some embodiments, both secretion of the pro-inflammatory cytokine and phagocytosis are increased by about 5% to about 50%, about 5% to about 40%, about 5% to about 30%, about 5% to about 20%, about 5% to about 10%, about 10% to about 50%, about 10% to about 40%, about 10% to about 30%, about 10% to about 20%, about 20% to about 50%, about 20% to about 40%, about 20% to about 30%, about 30% to about 50%, about 30% to about 40%, or about 40% to about 50%, relative to an otherwise identical CAR having an intracellular domain consisting of a CD3ζ intracellular signaling domain. In some embodiments, both secretion of the pro-inflammatory cytokine and phagocytosis are increased by at least 5%, relative to an otherwise identical CAR having an intracellular domain consisting of a CD3ζ intracellular signaling domain. In some embodiments, both secretion of the pro-inflammatory cytokine and phagocytosis are increased by at least 10%, relative to an otherwise identical CAR having an intracellular domain consisting of a CD3ζ intracellular signaling domain. In some embodiments, both secretion of the pro-inflammatory cytokine and phagocytosis are increased by at least 20%, relative to an otherwise identical CAR having an intracellular domain consisting of a CD3ζ intracellular signaling domain. In some embodiments, both secretion of the pro-inflammatory cytokine and phagocytosis are increased by at least 30%, relative to an otherwise identical CAR having an intracellular domain consisting of a CD3ζ intracellular signaling domain. In some embodiments, both secretion of the pro-inflammatory cytokine and phagocytosis are increased by at least 40%, relative to an otherwise identical CAR having an intracellular domain consisting of a CD3ζ intracellular signaling domain. In some embodiments, both secretion of the pro-inflammatory cytokine and phagocytosis are increased by at least 50%, relative to an otherwise identical CAR having an intracellular domain consisting of a CD3ζ intracellular signaling domain. In some embodiments, both secretion of the pro-inflammatory cytokine and phagocytosis are increased by more than about 50%, relative to an otherwise identical CAR having an intracellular domain consisting of a CD3ζ intracellular signaling domain.

In some embodiments, the increase in activity of the macrophage expressing a CAR provided herein is an increase in secretion of a pro-inflammatory cytokine and no increase in phagocytosis, relative to an otherwise identical CAR having an intracellular domain consisting of a CD3ζ intracellular signaling domain. In some embodiments, secretion of the pro-inflammatory cytokine is increased by about 5% to about 50%, about 5% to about 40%, about 5% to about 30%, about 5% to about 20%, about 5% to about 10%, about 10% to about 50%, about 10% to about 40%, about 10% to about 30%, about 10% to about 20%, about 20% to about 50%, about 20% to about 40%, about 20% to about 30%, about 30% to about 50%, about 30% to about 40%, or about 40% to about 50%, and there is no increase in phagocytosis, relative to an otherwise identical CAR having an intracellular domain consisting of a CD3ζ intracellular signaling domain. In some embodiments, secretion of the pro-inflammatory cytokine is increased by at least 5%, and there is no increase in phagocytosis, relative to an otherwise identical CAR having an intracellular domain consisting of a CD3ζ intracellular signaling domain. In some embodiments, secretion of the pro-inflammatory cytokine is increased by at least 10%, and there is no increase in phagocytosis, relative to an otherwise identical CAR having an intracellular domain consisting of a CD3ζ intracellular signaling domain. In some embodiments, secretion of the pro-inflammatory cytokine is increased by at least 20%, and there is no increase in phagocytosis, relative to an otherwise identical CAR having an intracellular domain consisting of a CD3ζ intracellular signaling domain. In some embodiments, secretion of the pro-inflammatory cytokine is increased by at least 30%, and there is no increase in phagocytosis, relative to an otherwise identical CAR having an intracellular domain consisting of a CD3ζ intracellular signaling domain. In some embodiments, secretion of the pro-inflammatory cytokine is increased by at least 40%, and there is no increase in phagocytosis, relative to an otherwise identical CAR having an intracellular domain consisting of a CD3ζ intracellular signaling domain. In some embodiments, secretion of the pro-inflammatory cytokine is increased by at least 50%, and there is no increase in phagocytosis, relative to an otherwise identical CAR having an intracellular domain consisting of a CD3ζ intracellular signaling domain. In some embodiments, secretion of the pro-inflammatory cytokine is increased by more than about 50%, and there is no increase in phagocytosis, relative to an otherwise identical CAR having an intracellular domain consisting of a CD3ζ intracellular signaling domain.

### 5.1.4.1. Non-Lymphoid Intracellular Signaling Domain

As provided herein, the present disclosure is based, in part, on the discovery that a CAR comprising a non-lymphoid intracellular signaling domain can enhance the activity of a macrophage, relative to an otherwise identical CAR having an intracellular domain consisting of a CD3ζ intracellular signaling domain. Accordingly, in specific embodiments, provided herein is a CAR comprising an intracellular domain that does not consist of a CD3ζ intracellular signaling domain. For example, in some embodiments, the intracellular signaling domain comprises a non-lymphoid intracellular signaling domain and a CD3ζ intracellular signaling domain. In other specific embodiments, the CAR comprises at least one intracellular domain that does not include an intracellular signaling domain derived from (a) the native B cell receptor complex (e.g. CD20, CD22, CD79a, CD79b), or (b) the native T cell receptor complex (e.g., CD3ζ, CD3δ, or CD3ε). In further embodiments, the CAR comprises at least one intracellular domain that does not include an intracellular signaling domain involved in the classical T cell signaling pathway, such as a co-stimulatory molecule from the CD28 family (e.g., CD28 or ICOS) or tumor necrosis factor receptor (TNFR) family (e.g., 4-1BB/CD137, OX40, or CD27).

Exemplary non-lymphoid intracellular signaling domains are provided in Table 3, below. In some embodiments, the non-lymphoid intracellular signaling domain is selected from the group consisting of BAI-1, CD86/B7-2, Lox1c, TIM4, MEGF10, SCARF1, CD93, DAP12, SLAMF7, IFNγR2, 2B4/CD244, DECTIN-1, CD206, DECTIN-3, CLEC2, and CD80/B7-1. In some embodiments, the non-lymphoid intracellular signaling domain is selected from the group consisting of CD86, Lox1c, 2B4, and DECTIN-1. In some embodiments, the non-lymphoid intracellular signaling domain is selected from the group consisting of CD80 and CD86. In some embodiments, the non-lymphoid intracellular signaling domain is not derived from a 2B4 intracellular signaling domain.

TABLE 3
Exemplary non-lymphoid intracellular signaling domains

| Intracellular | | SEQ |
|---|---|---|
| Signaling Domain | Sequence | ID NO |
| CD86 | KWKKKKRPRNSYKCGTNTMEREESEQTKKREKIHIPERSDEAQRVFKS | 40 |
| | SKTSSCDKSDTCF | |
| LOX1 | CWWPSYLFQLGKAKKGNSKEDPQDKVTQIKLDDFTM | 41 |
| 2B4 | WRRKRKEKQSETSPKEFLTIYEDVKDLKTRRNHEQEQTFPGGGSTIYS | 42 |
| | MIQSQSSAPTS | |
| DECTIN-1 | RWPPSAACSGKESVVAIRTNSQSDFHLQTYGDEDLNELDPHYEM | 43 |
| DECTIN-2 | LRLSLWGRKETSQPQQEQMM | 44 |
| TIM1 | KKYFFKKEVQQLSVSFSSLQIKALQNAVEKEVQAEDNIYIENSLYATD | 45 |
| CD40 | KKVAKKPTNKAPHPKQEPQEINFPDDLPGSNTAAPVQETLHGCQPVTQ | 46 |
| | EDGKESRISVQERQ | |
| CD11b | KLGFFKRQYKDMMSEGGPPGAEPQ | 47 |
| CSF1R | KYKQKPKYQVRWKIIESYEGNSYTFIDPTQLPYNEKWEFPRNNLQFGK | 48 |
| | TLGAGAFGKVVEATAFGLGKEDAVLKVAVKMLKSTAHADEKEALMSEL | |
| | KIMSHLGQHENIVNLLGACTHGGPVLVITEYCCYGDLLNFLRRKAEAM | |
| | LGPSLSPGQDPEGGVDYKNIHLEKKYVRRDSGFSSQGVDTYVEMRPVS | |
| | TSSNDSFSEQDLDKEDGRPLELRDLLHFSSQVAQGMAFLASKNCIHRD | |
| | VAARNVLLTNGHVAKIGDFGLARDIMNDSNYIVKGNARLPVKWMAPES | |
| | IFDCVYTVQSDVWSYGILLWEIFSLGLNPYPGILVNSKFYKLVKDGYQ | |
| | MAQPAFAPKNIYSIMQACWALEPTHRPTFQQICSFLQEQAQEDRRERD | |
| | YTNLPSSSRSGGSGSSSSELEEESSSEHLTCCEQGDIAQPLLQPNNYQ | |
| | FC | |
| CSF2R | KRFLRIQRLFPPVPQIKDKLNDNHEVEDEIIWEEFTPEEGKGYREEVL | 49 |
| | TVKEIT | |
| CSF3R | SPNRKNPLWPSVPDPAHSSLGSWVPTIMEEDAFQLPGLGTPPITKLTV | 50 |
| | LEEDEKKPVPWESHNSSETCGLPTLVQTYVLQGDPRAVSTQPQSQSGT | |
| | SDQVLYGQLLGSPTSPGPGHYLRCDSTQPLLAGLTPSPKSYENLWFQA | |
| | SPLGTLVTPAPSQEDDCVFGPLLNFPLLQGIRVHGMEALGSF | |
| IL1R | KIFKIDIVLWYRDSCYDFLPIKASDGKTYDAYILYPKTVGEGSTSDCD | 51 |
| | IFVFKVLPEVLEKQCGYKLFIYGRDDYVGEDIVEVINENVKKSRRLII | |
| | ILVRETSGFSWLGGSSEEQIAMYNALVQDGIKVVLLELEKIQDYEKMP | |
| | ESIKFIKQKHGAIRWSGDFTQGPQSAKTRFWKNVRYHMPVQRRSPSSK | |
| | HQLLSPATKEKLQREAHVPLG | |
| TLR1 | SYLDLPWYLRMVCQWTQTRRRARNIPLEELQRNLQFHAFISYSGHDSF | 52 |
| | WVKNELLPNLEKEGMQICLHERNFVPGKSIVENIITCIEKSYKSIFVL | |
| | SPNFVQSEWCHYELYFAHHNLFHEGSNSLILILLEPIPQYSIPSSYHK | |
| | LKSLMARRTYLEWPKEKSKRGLFWANLRAAINIKLTEQAKK | |
| TLR2 | HRFHGLWYMKMMWAWLQAKRKPRKAPSRNICYDAFVSYSERDAYWVEN | 53 |
| | LMVQELENFNPPFKLCLHKRDFIPGKWIIDNIIDSIEKSHKTVFVLSE | |
| | NFVKSEWCKYELDFSHFRLFDENNDAAILILLEPIEKKAIPQRFCKLR | |
| | KIMNTKTYLEWPMDEAQREGFWVNLRAAIKS | |
| TLR2 | CHRFHGLWYMKMMWAWLQAKRKPRKAPSRNICYDAFVSYSERDAYWVE | 54 |
| | NLMVQELENFNPPFKLCLHKRDFIPGKWIIDNIIDSIEKSHKTVFVLS | |
| | ENFVKSEWCKYELDFSHFRLFDENNDAAILILLEPIEKKAIPQRFCKL | |
| | RKIMNTKTYLEWPMDEAQREGFWVNLRAAIKS | |
| TLR3 | FEGWRISFYWNVSVHRVLGFKEIDRQTEQFEYAAYIIHAYKDKDWVWE | 55 |
| | HFSSMEKEDQSLKFCLEERDFEAGVFELEAIVNSIKRSRKIIFVITHH | |
| | LLKDPLCKRFKVHHAVQQAIEQNLDSIILVFLEEIPDYKLNHALCLRR | |
| | GMFKSHCILNWPVQKERIGAFRHKLQVALGSKNSVH | |
| TLR4 | KFYFHLMLLAGCIKYGRGENIYDAFVIYSSQDEDWVRNELVKNLEEGV | 56 |
| | PPFQLCLHYRDFIPGVAIAANIIHEGFHKSRKVIVVVSQHFIQSRWCI | |
| | FEYEIAQTWQFLSSRAGIIFIVLQKVEKTLLRQQVELYRLLSRNTYLE | |
| | WEDSVLGRHIFWRRLRKALLDGKSWNPEGTVGTGCNWQEATSI | |
| TLR5 | TKFRGFCFICYKTAQRLVFKDHPQGTEPDMYKYDAYLCFSSKDFTWVQ | 57 |
| | NALLKHLDTQYSDQNRFNLCFEERDFVPGENRIANIQDAIWNSRKIVC | |
| | LVSRHFLRDGWCLEAFSYAQGRCLSDLNSALIMVVVGSLSQYQLMKHQ | |
| | SIRGFVQKQQYLRWPEDFQDVGWFLHKLSQQILKKEKEKKKDNNIPLQ | |
| | TVATIS | |
| TLR6 | YLDLPWYLRMVCQWTQTRRRARNIPLEELQRNLQFHAFISYSEHDSAW | 58 |
| | VKSELVPYLEKEDIQICLHERNFVPGKSIVENIINCIEKSYKSIFVLS | |
| | PNFVQSEWCHYELYFAHHNLFHEGSNNLILILLEPIPQNSIPNKYHKL | |
| | KALMTQRTYLQWPKEKSKRGLFWANIRAAFNMKLTLVTENNDVKS | |
| TLR7 | HLYFWDVWYIYHFCKAKIKGYQRLISPDCCYDAFIVYDTKDPAVTEWV | 59 |
| | LAELVAKLEDPREKHFNLCLEERDWLPGQPVLENLSQSIQLSKKTVFV | |
| | MTDKYAKTENFKIAFYLSHQRLMDEKVDVIILIFLEKPFQKSKFLQLR | |
| | KRLCGSSVLEWPTNPQAHPYFWQCLKNALATDNHVAYSQVFKETV | |
| TLR8 | AHHLFYWDVWFIYNVCLAKVKGYRSLSTSQTFYDAYISYDTKDASVTD | 60 |
| | WVINELRYHLEESRDKNVLLCLEERDWDPGLAIIDNLMQSINQSKKTV | |
| | FVLTKKYAKSWNFKTAFYLALQRLMDENMDVIIFILLEPVLQHSQYLR | |
| | LRQRICKSSILQWPDNPKAEGLFWQTLRNVVLTENDSRYNNMYVDSIK | |
| | QY | |
| SCARF1 | CCCWAPRSDLKDRPARDGATVSRMKLQVWGTLTSLGSTLPCRSLSSHK | 61 |
| | LPWVTVSHHDPEVPFNHSFIEPPSAGWATDDSFSSDPESGEADEVPAY | |
| | CVPPQEGMVPVAQAGSSEASLAAGAFPPPEDASTPFAIPRTSSLARAK | |
| | RPSVSFAEGTKFAPQSRRSSGELSSPLRKPKRLSRGAQSGPEGREAEE | |
| | STGPEEAEAPESFPAAASPGDSATGHRRPPLGGRTVAEHVEAIEGSVQ | |
| | ESSGPVTTIYMLAGKPRGSEGPVRSVFRHFGSFQKGQAEAKVKRAIPK | |
| | PPRQALNRKKGSPGLASGSVGQSPNSAPKAGLPGATGPMAVRPEEAVR | |
| | GLGAGTESSRRAQEPVSGCGSPEQDPQKQAEEERQEEPEYENVVPISR | |
| | PPEP | |
| SCARF2 | CCCACRGKDPTRRPRPRRELSLGRKKAPHRLCGRFSRISMKLPRIPLR | 62 |
| | RQKLPKVVVAHHDLDNTLNCSFLEPPSGLEQPSPSWSSRASFSSFDTT | |
| | DEGPVYCVPHEEAPAESRDPEVPTVPAEAPAPSPVPLTTPASAEEAIP | |
| | LPASSDSERSASSVEGPGGALYARVARREARPARARGEIGGLSLSPSP | |
| | ERRKPPPPDPATKPKVSWIHGKHSAAAAGRAPSPPPPGSEAAPSPSKR | |
| | KRTPSDKSAHTVEHGSPRTRDPTPRPPGLPEEATALAAPSPPRARARG | |
| | RGPGLLEPTDAGGPPRSAPEAASMLAAELRGKTRSLGRAEVALGAQGP | |
| | REKPAPPQKAKRSVPPASPARAPPATETPGPEKAATDLPAPETPRKKT | |
| | PIQKPPRKKSREAAGELGRAGAPTL | |
| DC-SIGN | CGALSKYGRTQRFGLGRLQEEELLGLQQLRPEKSDSM | 63 |
| CXCL16 | CKRRRGQSPQSSPDLPVHYIPVAPDSNT | 64 |
| CD93 | GLLVYRKRRAKREEKKEKKPQNAADSYSWVPERAESRAMENQYSPTPG | 65 |
| | TDC | |
| LIMPII | GQGSMDEGTADERAPLIRT | 66 |
| MELLEC | LAVPRWTSSPARHETRRPEPHRTTASGQSHLSMTTDGDDDLMDRTSSY | 67 |
| | KAQM | |
| DNGR1 | LCCAGSCKNSSLCKQYTDPAPSDWQLSTYIEEEHM | 68 |
| TIM4 | LRGKLMETYCSQKHTRLDYIGDSKNVLNDVQHGREDEDGLFTL | 69 |
| FCER1G | LRKRRDSLSLSTQRTQGPAESARNLEYVSVSPTNNTVYASVTHSNRET | 70 |
| | EIWTPRENDTITIYSTINHSKESKPTFSRATALDNV | |
| VEGFR2 | LRTVKRANGGELKTGYLSIVMDPDELPLDEHCERLPYDASKWEFPRDR | 71 |
| | LKLGKPLGRGAFGQVIEADAFGIDKTATCRTVAVKMLKEGATHSEHRA | |
| | LMSELKILIHIGHHLNVVNLLGACTKPGGPLMVIVEFCKFGNLSTYLR | |
| | SKRNEFVPYKTKGARFRQGKDYVGAIPVDLKRRLDSITSSQSSASSGF | |
| | VEEKSLSDVEEEEAPEDLYKDFLTLEHLICYSFQVAKGMEFLASRKCI | |
| | HRDLAARNILLSEKNVVKICDFGLARDIYKDPDYVRKGDARLPLKWMA | |
| | PETIFDRVYTIQSDVWSFGVLLWEIFSLGASPYPGVKIDEEFCRRLKE | |
| | GTRMRAPDYTTPEMYQTMLDCWHGEPSQRPTFSELVEHLGNLLQANAQ | |
| | QDGKDYIVLPISETLSMEEDSGLSLPTSPVSCMEEEEVCDPKFHYDNT | |
| | AGISQYLQNSKRKSRPVSVKTFEDIPLEEPEVKVIPDDNQTDSGMVLA | |
| | SEELKTLEDRTKLSPSFGGMVPSKSRESVASEGSNQTSGYQSGYHSDD | |
| | TDTTVYSSEEAELLKLIEIGVQTGSTAQILQPDSGTTLSSPPV | |
| IFNγR2 | LVLKYRGLIKYWFHTPPSIPLQIEEYLKDPTQPILEALDKDSSPKDDV | 72 |
| | WDSVSIISFPEKEQEDVLQTL | |
| CLEC2 | MVRWWSSSASGVSILAPKRTKINLTIYGDEDQM | 73 |
| MARCO | NVGNRRKPKPVNIEFPEMAIQHFAAQQTESLLEDEKLIKKNRM | 74 |
| DEC-205 | QRHRLHLAGFSSVRYAQGVNEDEIMLPSFHD | 75 |
| RAGE | QRRQRRGEERKAPENQEEEEERAELNQSEEPEAGESSTGGP | 76 |
| STAB1 | RARGKPMGFGFSAFQAEDDADDDFSPWQEGTNPTLVSVPNPVFGSDTF | 77 |
| | CEPFDDSLLEEDFPDTQRILTVK | |
| CD28 | RSKRSRLLHSDYMNMTPRRPGPTRKHYQPYAPPRDFAAYRS | 78 |
| LANGERIN | RVTPTKGPVLSPGSKPPPERPWLSINQKDVTFHADPAEKEVTM | 79 |
| CD36 | SYCACRSKTIK | 80 |
| CD80 | TTYCFAPRCRERRRNERLRRESVRPV | 81 |
| CD80 | TYCFAPRCRERRRNERLRRESVRPV | 82 |
| SLAMF7 | WFLKRERQEEYIEEKKRVDICRETPNICPHSGENTEYDTIPHTNRTIL | 83 |
| | KEDPANTVYSTVEIPKKMENPHSLLTMPDTPRLFAYENVI | |
| DAP12 | YFLGRLVPRGRGAAEAATRKQRITETESPYQELQGQRSDVYSDLNTQR | 84 |
| | PYYK | |
| MRC1/CD206 | YKKRRVHLPQEGAFENTLYFNSQSSPGTSDMKDLVGNIEQNEHSVI | 85 |
| MEGF10 | YRHKQKGKESSMPAVTYTPAMRVVNADYTISGTLPHSNGGNANSHYFT | 86 |
| | NPSYHTLTQCATSPHVNNRDRMTVTKSKNNQLFVNLKNVNPGKRGPVG | |
| | DCTGTLPADWKHGGYLNELGAFGLDRSYMGKSLKDLGKNSEYNSSNCS | |
| | LSSSENPYATIKDPPVLIPKSSECGYVEMKSPARRDSPYAEINNSTSA | |
| | NRNVYEVEPTVSVVQGVFSNNGRLSQDPYDLPKNSHIPCHYDLLPVRD | |
| | SSSSPKQEDSGGSSSNSSSSSE | |
| STABILIN2 | YSYFRINRRTIGFQHFESEEDINVAALGKQQPENISNPLYESTTSAPP | 87 |
| | EPSYDPFTDSEERQLEGNDPLRTL | |
| BAI-1 | YWSMRLQARGGPSPLKSNSDSARLPISSGSTSSSRI | 88 |

In specific embodiments, a CAR provided herein comprises an intracellular signaling domain comprising an amino acid sequence at least 90%, at least 95%, or 100% identical to an amino acid sequence provided in Table 3. In specific embodiments, a CAR provided herein comprises a transmembrane domain comprising an amino acid sequence at least 90%, at least 95%, or 100% identical to SEQ ID NO:40, SEQ ID NO:41, SEQ ID NO:42, or SEQ ID NO:43. In some embodiments, a CAR provided herein comprises an intracellular signaling domain comprising an amino acid sequence at least 90%, at least 95%, or 100% identical to SEQ ID NO: 40. In some embodiments, a CAR provided herein comprises an intracellular signaling domain comprising an amino acid sequence at least 90%, at least 95%, or 100% identical to SEQ ID NO: 41. In some embodiments, a CAR provided herein comprises an intracellular signaling domain comprising an amino acid sequence at least 90%, at least 95%, or 100% identical to SEQ ID NO: 42. In some embodiments, a CAR provided herein comprises an intracellular signaling domain comprising an amino acid sequence at least 90%, at least 95%, or 100% identical to SEQ ID NO: 43. In some embodiments, a CAR provided herein comprises an intracellular signaling domain comprising an amino acid sequence at least 90%, at least 95%, or 100% identical to SEQ ID NO: 44. In some embodiments, a CAR provided herein comprises an intracellular signaling domain comprising an amino acid sequence at least 90%, at least 95%, or 100% identical to SEQ ID NO: 45. In some embodiments, a CAR provided herein comprises an intracellular signaling domain comprising an amino acid sequence at least 90%, at least 95%, or 100% identical to SEQ ID NO: 46. In some embodiments, a CAR provided herein comprises an intracellular signaling domain comprising an amino acid sequence at least 90%, at least 95%, or 100% identical to SEQ ID NO: 47. In some embodiments, a CAR provided herein comprises an intracellular signaling domain comprising an amino acid sequence at least 90%, at least 95%, or 100% identical to SEQ ID NO: 48. In some embodiments, a CAR provided herein comprises an intracellular signaling domain comprising an amino acid sequence at least 90%, at least 95%, or 100% identical to SEQ ID NO: 49. In some embodiments, a CAR provided herein comprises an intracellular signaling domain comprising an amino acid sequence at least 90%, at least 95%, or 100% identical to SEQ ID NO: 50. In some embodiments, a CAR provided herein comprises an intracellular signaling domain comprising an amino acid sequence at least 90%, at least 95%, or 100% identical to SEQ ID NO: 51. In some embodiments, a CAR provided herein comprises an intracellular signaling domain comprising an amino acid sequence at least 90%, at least 95%, or 100% identical to SEQ ID NO: 52. In some embodiments, a CAR provided herein comprises an intracellular signaling domain comprising an amino acid sequence at least 90%, at least 95%, or 100% identical to SEQ ID NO: 53. In some embodiments, a CAR provided herein comprises an intracellular signaling domain comprising an amino acid sequence at least 90%, at least 95%, or 100% identical to SEQ ID NO: 54. In some embodiments, a CAR provided herein comprises an intracellular signaling domain comprising an amino acid sequence at least 90%, at least 95%, or 100% identical to SEQ ID NO: 55. In some embodiments, a CAR provided herein comprises an intracellular signaling domain comprising an amino acid sequence at least 90%, at least 95%, or 100% identical to SEQ ID NO: 56. In some embodiments, a CAR provided herein comprises an intracellular signaling domain comprising an amino acid sequence at least 90%, at least 95%, or 100% identical to SEQ ID NO: 57. In some embodiments, a CAR provided herein comprises an intracellular signaling domain comprising an amino acid sequence at least 90%, at least 95%, or 100% identical to SEQ ID NO: 58. In some embodiments, a CAR provided herein comprises an intracellular signaling domain comprising an amino acid sequence at least 90%, at least 95%, or 100% identical to SEQ ID NO: 59. In some embodiments, a CAR provided herein comprises an intracellular signaling domain comprising an amino acid sequence at least 90%, at least 95%, or 100% identical to SEQ ID NO: 60. In some embodiments, a CAR provided herein comprises an intracellular signaling domain comprising an amino acid sequence at least 90%, at least 95%, or 100% identical to SEQ ID NO: 61. In some embodiments, a CAR provided herein comprises an intracellular signaling domain comprising an amino acid sequence at least 90%, at least 95%, or 100% identical to SEQ ID NO: 62. In some embodiments, a CAR provided herein comprises an intracellular signaling domain comprising an amino acid sequence at least 90%, at least 95%, or 100% identical to SEQ ID NO: 63. In some embodiments, a CAR provided herein comprises an intracellular signaling domain comprising an amino acid sequence at least 90%, at least 95%, or 100% identical to SEQ ID NO: 64. In some embodiments, a CAR provided herein comprises an intracellular signaling domain comprising an amino acid sequence at least 90%, at least 95%, or 100% identical to SEQ ID NO: 65. In some embodiments, a CAR provided herein comprises an intracellular signaling domain comprising an amino acid sequence at least 90%, at least 95%, or 100% identical to SEQ ID NO: 66. In some embodiments, a CAR provided herein comprises an intracellular signaling domain comprising an amino acid sequence at least 90%, at least 95%, or 100% identical to SEQ ID NO: 67. In some embodiments, a CAR provided herein comprises an intracellular signaling domain comprising an amino acid sequence at least 90%, at least 95%, or 100% identical to SEQ ID NO: 68. In some embodiments, a CAR provided herein comprises an intracellular signaling domain comprising an amino acid sequence at least 90%, at least 95%, or 100% identical to SEQ ID NO: 69. In some embodiments, a CAR provided herein comprises an intracellular signaling domain comprising an amino acid sequence at least 90%, at least 95%, or 100% identical to SEQ ID NO: 70. In some embodiments, a CAR provided herein comprises an intracellular signaling domain comprising an amino acid sequence at least 90%, at least 95%, or 100% identical to SEQ ID NO: 71. In some embodiments, a CAR provided herein comprises an intracellular signaling domain comprising an amino acid sequence at least 90%, at least 95%, or 100% identical to SEQ ID NO: 72. In some embodiments, a CAR provided herein comprises an intracellular signaling domain comprising an amino acid sequence at least 90%, at least 95%, or 100% identical to SEQ ID NO: 73. In some embodiments, a CAR provided herein comprises an intracellular signaling domain comprising an amino acid sequence at least 90%, at least 95%, or 100% identical to SEQ ID NO: 74. In some embodiments, a CAR provided herein comprises an intracellular signaling domain comprising an amino acid sequence at least 90%, at least 95%, or 100% identical to SEQ ID NO: 75. In some embodiments, a CAR provided herein comprises an intracellular signaling domain comprising an amino acid sequence at least 90%, at least 95%, or 100% identical to SEQ ID NO: 76. In some embodiments, a CAR provided herein comprises an intracellular signaling domain comprising an amino acid sequence at least 90%, at least 95%, or 100% identical to SEQ ID NO: 77. In some embodiments, a CAR provided herein comprises an intracellular signaling domain comprising an amino acid sequence at least 90%, at least 95%, or 100% identical to SEQ ID NO: 78. In some embodiments, a CAR provided herein comprises an intracellular signaling domain comprising an amino acid sequence at least 90%, at least 95%, or 100% identical to SEQ ID NO: 79. In some embodiments, a CAR provided herein comprises an intracellular signaling domain comprising an amino acid sequence at least 90%, at least 95%, or 100% identical to SEQ ID NO: 80. In some embodiments, a CAR provided herein comprises an intracellular signaling domain comprising an amino acid sequence at least 90%, at least 95%, or 100% identical to SEQ ID NO: 81. In some embodiments, a CAR provided herein comprises an intracellular signaling domain comprising an amino acid sequence at least 90%, at least 95%, or 100% identical to SEQ ID NO: 82. In some embodiments, a CAR provided herein comprises an intracellular signaling domain comprising an amino acid sequence at least 90%, at least 95%, or 100% identical to SEQ ID NO: 83. In some embodiments, a CAR provided herein comprises an intracellular signaling domain comprising an amino acid sequence at least 90%, at least 95%, or 100% identical to SEQ ID NO: 84. In some embodiments, a CAR provided herein comprises an intracellular signaling domain comprising an amino acid sequence at least 90%, at least 95%, or 100% identical to SEQ ID NO: 85. In some embodiments, a CAR provided herein comprises an intracellular signaling domain comprising an amino acid sequence at least 90%, at least 95%, or 100% identical to SEQ ID NO: 86. In some embodiments, a CAR provided herein comprises an intracellular signaling domain comprising an amino acid sequence at least 90%, at least 95%, or 100% identical to SEQ ID NO: 87. In some embodiments, a CAR provided herein comprises an intracellular signaling domain comprising an amino acid sequence at least 90%, at least 95%, or 100% identical to SEQ ID NO: 88. In some embodiments, a CAR provided herein comprises an intracellular signaling domain comprising an amino acid sequence at least 90%, at least 95%, or 100% identical to SEQ ID NO: 81 or SEQ ID NO:82.

In certain embodiments, a CAR provided herein does not comprise an intracellular signaling domain that comprises a TLR intracellular signaling domain, e.g., a TLR1, TLR2, TLR3, TLR4, TLR5, TLR6, TLR7 or TLR8 intracellular signaling domain.

### 5.1.4.2. ITAM-Containing Intracellular Signaling Domain

As provided herein, the present disclosure relates, in part, to a CAR having at least two intracellular signaling domains (e.g., a first intracellular signaling domain, such as a non-lymphoid intracellular signaling domain described in Section 5.1.4.1, and a second intracellular signaling domain comprising one or more immune-receptor-tyrosine-based-activation-motifs (ITAMs)). Exemplary ITAM-containing intracellular signaling domains are provided in Table 4.

In specific embodiments, a CAR provided herein comprises an intracellular signaling domain comprising an amino acid sequence at least 90%, at least 95%, or 100% identical to an amino acid sequence provided in Table 4. In specific embodiments, a CAR provided herein comprises a transmembrane domain comprising an amino acid sequence at least 90%, at least 95%, or 100% identical to SEQ ID NO:89, or SEQ ID NO:92. In some embodiments, a CAR provided herein comprises an intracellular signaling domain comprising an amino acid sequence at least 90%, at least 95%, or 100% identical to SEQ ID NO: 89. In some embodiments, a CAR provided herein comprises an intracellular signaling domain comprising an amino acid sequence at least 90%, at least 95%, or 100% identical to SEQ ID NO: 90. In some embodiments, a CAR provided herein comprises an intracellular signaling domain comprising an amino acid sequence at least 90%, at least 95%, or 100% identical to SEQ ID NO: 91. In some embodiments, a CAR provided herein comprises an intracellular signaling domain comprising an amino acid sequence at least 90%, at least 95%, or 100% identical to SEQ ID NO: 92.

TABLE 4
Exemplary ITAM containing intracellular signaling domains

| Intracellular | | SEQ |
|---|---|---|
| Signaling Domain | Sequence | ID NO |
| CD3ζ | RVKFSRSADAPAYQQGQNQLYNELNLGRREEYDVLDKRRGRDPEMGG | 89 |
| | KPQRRKNPQEGLYNELQKDKMAEAYSEIGMKGERRRGKGHDGLYQGL | |
| | STATKDTYDALHMQALPPR | |
| CD3ϵ | KNRKAKAKPVTRGAGAGGRQRGQNKERPPPVPNPDYEPRKGQRDLY | 90 |
| | SGLNQRRI | |
| CD3δ | GHETGRLSGAADTQALLRNDQVYQPLRDRDDAQYSHLGGNWARNK | 91 |
| FcγRI | RKELKRKKKWDLEISLDSGHEKKVISSLQEDRHLEEELKCQEQKEEQ | 92 |
| | LQEGVHRKEPQGAT | |

In some embodiments, a CAR provided herein comprises an intracellular domain having a CD86 intracellular signaling domain and a CD3ζ intracellular signaling domain. In specific embodiments, a CAR provided herein comprises an intracellular domain having a CD86 intracellular signaling domain comprising an amino acid sequence of SEQ ID NO:40 and a CD3ζ intracellular signaling domain comprising an amino acid sequence of SEQ ID NO:89. In some embodiments, a CAR provided herein comprises an intracellular domain having a 2B4 intracellular signaling domain and a CD3ζ intracellular signaling domain. In some embodiments, a CAR provided herein does not comprise an intracellular domain having a 2B4 intracellular signaling domain and a CD3ζ intracellular signaling domain. In specific embodiments, a CAR provided herein comprises an intracellular domain having a 2B4 intracellular signaling domain comprising an amino acid sequence of SEQ ID NO:42 and a CD3ζ intracellular signaling domain comprising an amino acid sequence of SEQ ID NO:89. In some embodiments, a CAR provided herein comprises an intracellular domain having a 2B4 intracellular signaling domain and a FcγRI intracellular signaling domain. In some embodiments, a CAR provided herein does not comprise an intracellular domain having a 2B4 intracellular signaling domain and a FcγRI intracellular signaling domain. In specific embodiments, a CAR provided herein comprises an intracellular domain having a 2B4 intracellular signaling domain comprising an amino acid sequence of SEQ ID NO:42 and a FcγRI intracellular signaling domain comprising an amino acid sequence of SEQ ID NO:92. In some embodiments, a CAR provided herein comprises an intracellular domain having a DECTIN-1 intracellular signaling domain and a CD3ç intracellular signaling domain. In specific embodiments, a CAR provided herein comprises an intracellular domain having a DECTIN-1 intracellular signaling domain comprising an amino acid sequence of SEQ ID NO:43 and a CD3ζ intracellular signaling domain comprising an amino acid sequence of SEQ ID NO:89. In some embodiments, a CAR provided herein comprises an intracellular domain having a DECTIN-1 intracellular signaling domain and a FcγRI intracellular signaling domain. In specific embodiments, a CAR provided herein comprises an intracellular domain having a DECTIN-1 intracellular signaling domain comprising an amino acid sequence of SEQ ID NO:43 and a FcγRI intracellular signaling domain comprising an amino acid sequence of SEQ ID NO:92. In some embodiments, a CAR provided herein comprises an intracellular domain having a Lox1c intracellular signaling domain and a CD3ζ intracellular signaling domain. In specific embodiments, a CAR provided herein comprises an intracellular domain having a Lox1c intracellular signaling domain comprising an amino acid sequence of SEQ ID NO:41 and a CD3ζ intracellular signaling domain comprising an amino acid sequence of SEQ ID NO:89. In some embodiments, a CAR provided herein comprises an intracellular domain having a Lox1c intracellular signaling domain and a FcγRI intracellular signaling domain. In specific embodiments, a CAR provided herein comprises an intracellular domain having a Lox1c intracellular signaling domain comprising an amino acid sequence of SEQ ID NO: 41 and a FcγRI intracellular signaling domain comprising an amino acid sequence of SEQ ID NO: 92. In some embodiments, a CAR provided herein comprises an intracellular domain having a CD80 intracellular signaling domain and a CD3ζ intracellular signaling domain. In specific embodiments, a CAR provided herein comprises an intracellular domain having a CD80 intracellular signaling domain comprising an amino acid sequence of SEQ ID NO:81 or SEQ ID NO: 82, and a CD3ζ intracellular signaling domain comprising an amino acid sequence of SEQ ID NO: 89. In specific embodiments, a CAR provided herein comprises an intracellular domain having a CD80 intracellular signaling domain comprising an amino acid sequence of SEQ ID NO: 81, and a CD3ζ intracellular signaling domain comprising an amino acid sequence of SEQ ID NO: 89. In specific embodiments, a CAR provided herein comprises an intracellular domain having a CD80 intracellular signaling domain comprising an amino acid sequence of SEQ ID SEQ ID NO:82, and a CD3ζ intracellular signaling domain comprising an amino acid sequence of SEQ ID NO:89. In some embodiments, a CAR provided herein comprises an intracellular domain having a CD80 intracellular signaling domain and a FcγRI intracellular signaling domain. In specific embodiments, a CAR provided herein comprises an intracellular domain having a CD80 intracellular signaling domain comprising an amino acid sequence of SEQ ID NO: 81 or SEQ ID NO:82 and a FcγRI intracellular signaling domain comprising an amino acid sequence of SEQ ID NO:92. In specific embodiments, a CAR provided herein comprises an intracellular domain having a CD80 intracellular signaling domain comprising an amino acid sequence of SEQ ID NO:81 and a FcγRI intracellular signaling domain comprising an amino acid sequence of SEQ ID NO:92. In specific embodiments, a CAR provided herein comprises an intracellular domain having a CD80 intracellular signaling domain comprising an amino acid sequence of SEQ ID NO:82 and a FcγRI intracellular signaling domain comprising an amino acid sequence of SEQ ID NO:92. In some embodiments, a CAR provided herein comprises an intracellular domain having a CD40 intracellular signaling domain and a CD3ζ intracellular signaling domain. In specific embodiments, a CAR provided herein comprises an intracellular domain having a CD40 intracellular signaling domain comprising an amino acid sequence of SEQ ID NO:46 and a CD3ζ intracellular signaling domain comprising an amino acid sequence of SEQ ID NO:89. In specific embodiments, a CAR provided herein does not comprise an intracellular domain having a CD86 intracellular signaling domain comprising an amino acid sequence of SEQ ID NO:40 and a FcγRI intracellular signaling domain comprising an amino acid sequence of SEQ ID NO:92.

### 5.2. Polynucleotides

In one aspect, provided herein are polynucleotides that encode a CAR of the present disclosure, such as a CAR described in Section 5.1. In some embodiments, the polynucleotide comprises DNA (e.g., cDNA). In some embodiments, the polynucleotide comprises RNA (e.g., mRNA). In certain embodiments, the polynucleotide is isolated. In certain embodiments, the polynucleotide is substantially pure.

In some embodiments, the polynucleotide provided herein encodes a CARs comprising an antigen recognition moiety (such as an antigen recognition moiety described in Section 5.1.1), a hinge domain (such as a hinge domain described in Section 5.1.2), a transmembrane domain (such as a transmembrane domain described in Section 5.1.3), and an intracellular domain (such as an intracellular domain described in Section 5.1.4), and the transmembrane domain and/or intracellular domain comprise domains derived from a receptor associated with macrophage activity. In some embodiments, the polynucleotide provided herein encodes a CARs comprising an antigen recognition moiety (such as an antigen recognition moiety described in Section 5.1.1), a hinge domain (such as a hinge domain described in Section 5.1.2), a transmembrane domain (such as a transmembrane domain described in Section 5.1.3), and an intracellular domain (such as an intracellular domain described in Section 5.1.4), and the transmembrane domain comprises a transmembrane domain derived from a receptor associated with macrophage activity. In some embodiments, the polynucleotide provided herein encodes a CARs comprising an antigen recognition moiety (such as an antigen recognition moiety described in Section 5.1.1), a hinge domain (such as a hinge domain described in Section 5.1.2), a transmembrane domain (such as a transmembrane domain described in Section 5.1.3), and an intracellular domain (such as an intracellular domain described in Section 5.1.4), and the intracellular domain comprises an intracellular domain derived from a receptor associated with macrophage activity. In some embodiments, the polynucleotide provided herein encodes a CARs comprising an antigen recognition moiety (such as an antigen recognition moiety described in Section 5.1.1), a hinge domain (such as a hinge domain described in Section 5.1.2), a transmembrane domain (such as a transmembrane domain described in Section 5.1.3), and an intracellular domain (such as an intracellular domain described in Section 5.1.4), and both the transmembrane domain and intracellular domain comprise domains derived from a receptor associated with macrophage activity. In some embodiments, the macrophage activity comprises secretion of a pro-inflammatory cytokine. In some embodiments, the macrophage activity comprises phagocytosis of a cell expressing a target antigen. In some embodiments, the macrophage activity comprises secretion of a pro-inflammatory cytokine and phagocytosis of a cell expressing a target antigen.

In some aspects, the chimeric antigen receptor (CAR) of the present disclosure comprises a signal peptide. In some embodiments, the signal peptide has a polynucleotide sequence of

(SEQ ID NO: 3)
ATGGCACTGCCAGTGACCGCCCTGCTGCTGCCTCTGGCCCTGCTGCTGCA
CGCAGCAAGGCCA.

In some embodiments, the signal peptide has a polynucleotide sequence with at least 80% sequence identity with SEQ ID NO: 3. In some embodiments, the signal peptide has a polynucleotide sequence with at least 90%, 95%, 97% or 99% sequence identity with SEQ ID NO: 3.

### 5.2.1. Promoter

In certain aspects, provided herein is a polynucleotide that encodes a CAR (such as a polynucleotide described in Section 5.2), wherein the polynucleotide is operatively linked to a promoter. In some embodiments, the promoter is a constitutively active promoter. Constitutively active promoters are known in the art, and any suitable constitutively active promoter capable of expressing the CAR in a mammalian cell, such as a mammalian cell described in Section 5.5, can be used. Non-limiting examples of a constitutively active promoter include, for example, the elongation factor-1 alpha (EF1α) promoter, the cytomegalovirus (CMV) promoter, the cytomegalovirus (CMV) enhancer fused to the chicken beta-actin (CAG) promoter, and the phosphoglycerate kinase (PGK) promoter. In some embodiments, the polynucleotide provided herein is operably linked to a EF1α promoter. In some embodiments, the polynucleotide provided herein is operably linked to a CAG promoter. In some embodiments, the polynucleotide provided herein is operably linked to a PGK promoter. In some embodiments, the polynucleotide provided herein is operably linked to a CAG promoter. In some embodiments, the polynucleotide provided herein is operably linked to a T7 promoter.

Also provided herein is a polynucleotide that encodes a CAR (such as a polynucleotide described in Section 5.2), wherein the polynucleotide is operatively linked to a tissue specific promoter. For example, the tissue specific promoter can be a promoter that selectively enhances or facilitates transcription of a gene in certain cell types (e.g., a monocyte and/or macrophage), but not in other cell types (e.g., a non-monotype or a non-macrophage). A representative non-myeloid reference cell may be a HeLa or a 293T cell.

In some embodiments, the promoter is a myeloid-specific promoter. As used herein, the term “myeloid specific promoter” is intended to mean a promoter that enhances or facilitates transcription of a gene in a myeloid cell (e.g., a macrophage or monocyte), relative to a non-myeloid cell as measured by, for example, a reporter gene assay (e.g., a luciferase, or chemiluminescent reporter assay) or measure of the downstream protein encoded by the gene. In some embodiments, the myeloid specific promoter enhances transcription of a reporter gene (e.g., GFP) in a monocyte or macrophage, relative to a constitutively active promoter (e.g., CMV), and the myeloid specific promoter does not enhance transcription of a reporter gene (e.g., GFP) in a non-myeloid reference cell, relative to a constitutively active promoter (e.g., CMV). A representative non-myeloid reference cell may be, for example, a human intestinal epithelial cell (e.g., Caco-2), a cervix epithelioid carcinoma cell (e.g., HeLa), a human embryonic kidney cell 293 (e.g., HEK-293 or 293T), a T lymphocyte (e.g., Jurkat), or a mouse osteoblast (e.g., Oct-1).

In some embodiments, the myeloid specific promoter enhances transcription in a macrophage and/or monocyte cell approximately 5-fold, relative to in a non-myeloid cell. In some embodiments, the myeloid specific promoter enhances transcription in a macrophage and/or monocyte cell approximately 10-fold, relative to in a non-myeloid cell. In some embodiments, the myeloid specific promoter enhances transcription in a macrophage and/or monocyte cell approximately 20-fold, relative to in a non-myeloid cell. In some embodiments, the myeloid specific promoter enhances transcription in a macrophage and/or monocyte cell approximately 50-fold, relative to in a non-myeloid cell. In some embodiments, the myeloid specific promoter enhances transcription in a macrophage and/or monocyte cell approximately 75-fold, relative to in a non-myeloid cell. In some embodiments, the myeloid specific promoter enhances transcription in a macrophage and/or monocyte cell approximately 100-fold, relative to in a non-myeloid cell. In some embodiments, the myeloid specific promoter enhances transcription in a macrophage and/or monocyte cell about 5-fold to about 100-fold, relative to in a non-myeloid cell. In some embodiments, the myeloid specific promoter enhances transcription in a macrophage and/or monocyte cell about 10-fold to about 100-fold, relative to in a non-myeloid cell. In some embodiments, the myeloid specific promoter enhances transcription in a macrophage and/or monocyte cell about 25-fold to about 100-fold, relative to in a non-myeloid cell. In some embodiments, the myeloid specific promoter enhances transcription in a macrophage and/or monocyte cell about 50-fold to about 100-fold, relative to in a non-myeloid cell.

In some embodiments, the myeloid specific promoter enhances transcription in a macrophage and/or monocyte cell approximately 5-fold, relative to a constitutively active promoter in the macrophage and/or monocyte cell. In some embodiments, the myeloid specific promoter enhances transcription in a macrophage and/or monocyte cell approximately 10-fold, relative to a constitutively active promoter in the macrophage and/or monocyte cell. In some embodiments, the myeloid specific promoter enhances transcription in a macrophage and/or monocyte cell approximately 20-fold, relative to a constitutively active promoter in the macrophage and/or monocyte cell. In some embodiments, the myeloid specific promoter enhances transcription in a macrophage and/or monocyte cell approximately 50-fold, relative to a constitutively active promoter in the macrophage and/or monocyte cell. In some embodiments, the myeloid specific promoter enhances transcription in a macrophage and/or monocyte cell approximately 75-fold, relative to a constitutively active promoter in the macrophage and/or monocyte cell. In some embodiments, the myeloid specific promoter enhances transcription in a macrophage and/or monocyte cell approximately 100-fold, relative to a constitutively active promoter in the macrophage and/or monocyte cell. In some embodiments, the myeloid specific promoter enhances transcription in a macrophage and/or monocyte cell about 5-fold to about 100-fold, relative to a constitutively active promoter in the macrophage and/or monocyte cell. In some embodiments, the myeloid specific promoter enhances transcription in a macrophage and/or monocyte cell about 10-fold to about 100-fold, relative to a constitutively active promoter in the macrophage and/or monocyte cell. In some embodiments, the myeloid specific promoter enhances transcription in a macrophage and/or monocyte cell about 25-fold to about 100-fold, relative to a constitutively active promoter in the macrophage and/or monocyte cell. In some embodiments, the myeloid specific promoter enhances transcription in a macrophage and/or monocyte cell about 50-fold to about 100-fold, relative to a constitutively active promoter in the macrophage and/or monocyte cell.

In some embodiments, the myeloid specific promoter is a native macrophage or a native monocyte promoter, or fragment thereof. For example, the promoter can include the full-length, or a fraction thereof, of the promoter of a gene that is expressed in monocytes and/or macrophages (e.g., CD36, CD68, CD11b, or CSF1R). In some embodiments, the promoter can include the full-length, or a fraction thereof, of the promoter of a gene that is selectively expressed in monocytes and/or macrophages relative to a non-monotype or a non-macrophage, such as, for example, a HeLa or 293T cell.

In some embodiments, the myeloid specific promoter is synthetic promoter. Techniques known to one of skill in the art or described herein (e.g., Section 5.9.3 or in the Examples) may be used to generate and screen synthetic promoters. For example, synthetic promoters can be generated by random ligation of myeloid/macrophage cis elements. In some embodiments, synthetic promoter is selected from the group consisting of synthetic promoter-146 (SP146) (GenBank: DQ107383.1), synthetic promoter-107 (SP107) (GenBank: DQ107382.1), and synthetic promoter-60 (SP60) (GenBank: DQ107381.1). In some embodiments, the promoter is a SP146 promoter. In some embodiments, the promoter is a SP107 promoter. In some embodiments, the promoter is a SP60 promoter. Exemplary promoter polynucleotide sequences are provided in Table 5.

In specific embodiments, a promotor provided herein comprises a promoter within any of the nucleotide sequences provided in Table 5. In particular embodiments, a promoter provided herein comprises a promote that is at least 90%, at least 95%, or 100% identical to a polynucleotide sequence provided in Table 5. In specific embodiments, a promoter provided herein comprises a promoter that is at least 90%, at least 95%, or 100% identical to SEQ ID NO: 122. In specific embodiments, a promoter provided herein comprises a promoter that is at least 90%, at least 95%, or 100% identical to SEQ ID NO:123. In specific embodiments, a promoter provided herein comprises a promoter that is at least 90%, at least 95%, or 100% identical to SEQ ID NO:124.

TABLE 5
Exemplary promoter polynucleotide sequences

| | | SEQ |
|---|---|---|
| Promoter | Sequence | ID NO |
| Synthetic promoter- | CTAGCGAGGGCGGACCAGAAAAGGAGAAGTAGGAGCCAAGATTTCCAAA | 122 |
| 146 (SP146) | CTCTGTGGTTGCCTTGCCAAGATTTCCAAACTCTGTGGTTGCCTTGCAG | |
| | AAAAGGAGAAGTAGGAGAAGCGACTTCCTCTTTCCAGAAGCGACTTCCT | |
| | CTTTCCAGAGGAAGAGGGCGGAGGCTCACAAGGCAACCACAGAGTTTGG | |
| | AAATCTTGGAAGCGACTTCCTCTTTCCAGCAGAAAAGGAGAAGTAGGAG | |
| | AAGCGACTTCCTCTTTCCAGGTCCGCCCTCG | |
| Synthetic promoter- | CTAGCGAGGGCGGACAAGCGACTTCCTCTTTCCAGCAGAAAAGGAGAAG | 123 |
| 107 (SP107) | TAGGAGCCAAGATTTCCAAACTCTGTGGTTGCCTTGAAGCGACTTCCTC | |
| | TTTCCAGAAGCGACTTCCTCTTTCCAGCAGAAAAGGAGAAGTAGGAGCT | |
| | CCTACTTCTCCTTTTCTGCCAAGATTTCCAAACTCTGTGGTTGCCTTGC | |
| | TCCTACTTCTCCTTTTCTGCAGAAAAGGAGAAGTAGGAGCTGGAAAGAG | |
| | GAAGTCGCTTGAGGGCGGACG | |
| Synthetic promoter- | CTAGCGTCCGCCCTCAGGAAGAGGGCGGAGGCTCACCAAGATTTCCAAA | 124 |
| 60 (SP60) | CTCTGTGGTTGCCTTGCCAAGATTTCCAAACTCTGTGGTTGCCTTGCCA | |
| | AGATTTCCAAACTCTGTGGTTGCCTTGTGAGCCTCCGCCCTCTTCCTAA | |
| | GCGACTTCCTCTTTCCAGGAGGGCGGACG | |

### 5.3. Vectors

In some aspects, provided herein is a vector comprising a polynucleotide encoding a CAR (such as a polynucleotide described in Section 5.2). In some embodiments, the vector is viral vector. In some embodiments, the viral vector is selected from the group consisting of an adenoviral vector, a lentiviral vector, and a retroviral vector. In specific embodiments, the vector is an adenoviral vector. In specific embodiments, the vector is a lentiviral vector. In specific embodiments, the vector is a retroviral vector.

In some embodiments, the vector comprises one or more selection markers. Non-limiting examples of a selection marker include, for example, drug resistance genes for G418 (neo), puromycin (pac), hygromycin B (hph), zeocin (zeo), blasticidin S (bsd), and histidinol (hisD), as well selection markers suitable for fluorescence-activated cell sorting (FACS), such as, green fluorescent protein (GFP), yellow fluorescent protein (YFP), mCherry, and cyan fluorescent protein (CFP).

In specific embodiments, the one or more selection markers are operably linked to a promoter that is different from the promoter that regulates expression of the CAR. In some embodiments, the one or more selection markers are operably linked to a promoter selected from the group consisting of EF1α, CAG, T7 and PGK. In some embodiments, the one or more selection markers are operably linked to a EF1α promoter. In some embodiments, the one or more selection markers are operably linked to a CAG promoter. In some embodiments, the one or more selection markers are operably linked to a PGK promoter. In some embodiments, the one or more selection markers are operably linked to a T7 promoter. In some embodiments, the vector comprises constitutive expression or for inducible expression. In some embodiments, the vector comprises constitutive expression. In some embodiments, the vector comprises inducible expression. The selection of promoters, e.g., strong, weak, tissue-specific, inducible and developmental-specific, is within the ordinary skill of the artisan.

In some embodiments, the vector comprises two or more selection markers that are muliticistronic. In some embodiments, one selection marker (e.g., a drug resistance gene) is upstream a 2A peptide (e.g., P2A, T2A, E2A and F2A) and another selection marker (e.g., a selection marker suitable for fluorescence-activated cell sorting (FACS)) is downstream the 2A peptide. In some embodiments, one selection marker (e.g., a selection marker suitable for fluorescence-activated cell sorting (FACS)) is upstream a 2A peptide (e.g., P2A, T2A, E2A and F2A) and another selection marker (e.g., a drug resistance gene) is downstream the 2A peptide. In some embodiments, one selection marker (e.g., a drug resistance gene) is upstream an Internal Ribosome Entry Site (IRES) element and another selection marker (e.g., a selection marker suitable for fluorescence-activated cell sorting (FACS)) is downstream the IRES element. In some embodiments, one selection marker (e.g., a selection marker suitable for fluorescence-activated cell sorting (FACS)) is upstream a IRES element and another selection marker (e.g., a drug resistance gene) is downstream the IRES element.

TABLE 6
2A peptides

| | | SEQ |
|---|---|---|
| Peptide | Amino acid sequence* | ID NO: |
| T2A | (GSG)EGRGSLLTCGDVEENPGP | 93 |
| P2A | (GSG)ATNFSLLKQAGDVEENPGP | 94 |
| E2A | (GSG)QCTNYALLKLAGDVESNPGP | 95 |
| F2A | (GSG)VKQTLNFDLLKLAGDVESNPGP | 96 |

*(GSG) residues can be added to the 5′ end of the peptide to improve cleavage efficiency.

In some embodiments, the vector is designed for transient expression, stable expression, or both. In some embodiments, the vector is designed for stable expression. In some embodiments, the vector is designed for transient expression.

### 5.4. Polypeptides

In one aspect, provided herein is a CAR (such as a CAR describe in Section 5.1) comprising an antigen recognition moiety polypeptide (such as an antigen recognition moiety described in Section 5.1.1), a hinge domain polypeptide (such as a hinge domain described in Section 5.1.2), a transmembrane domain polypeptide (such as a transmembrane domain described in Section 5.1.3), and an intracellular domain polypeptide (such as an intracellular domain described in Section 5.1.4), and the transmembrane domain polypeptide and/or intracellular domain polypeptide comprise domains derived from a receptor associated with macrophage activity. In some embodiments, the CAR provided herein comprises an antigen recognition moiety polypeptide (such as an antigen recognition moiety described in Section 5.1.1), a hinge domain polypeptide (such as a hinge domain described in Section 5.1.2), a transmembrane domain polypeptide (such as a transmembrane domain described in Section 5.1.3), and an intracellular domain polypeptide (such as an intracellular domain described in Section 5.1.4), and the transmembrane domain polypeptide comprises a transmembrane domain derived from a receptor associated with macrophage activity. In some embodiments, the CAR provided herein comprises an antigen recognition moiety polypeptide (such as an antigen recognition moiety described in Section 5.1.1), a hinge domain polypeptide (such as a hinge domain described in Section 5.1.2), a transmembrane domain polypeptide (such as a transmembrane domain described in Section 5.1.3), and an intracellular domain polypeptide (such as an intracellular domain described in Section 5.1.4), and the intracellular domain polypeptide comprises an intracellular domain derived from a receptor associated with macrophage activity. In some embodiments, the CAR provided herein comprises an antigen recognition moiety polypeptide (such as an antigen recognition moiety described in Section 5.1.1), a hinge domain polypeptide (such as a hinge domain described in Section 5.1.2), a transmembrane domain polypeptide (such as a transmembrane domain described in Section 5.1.3), and an intracellular domain polypeptide (such as an intracellular domain described in Section 5.1.4), and both the transmembrane domain polypeptide and intracellular domain polypeptide comprise domains derived from a receptor associated with macrophage activity. In some embodiments, the macrophage activity comprises secretion of a pro-inflammatory cytokine. In some embodiments, the macrophage activity comprises phagocytosis of a cell expressing a target antigen. In some embodiments, the macrophage activity comprises secretion of a pro-inflammatory cytokine and phagocytosis of a cell expressing a target antigen.

In some embodiments, provided herein is a CAR comprising: (a) an antigen recognition moiety polypeptide; (b) a CD8 hinge domain polypeptide; (c) a transmembrane domain polypeptide, wherein the transmembrane domain polypeptide is selected from the group consisting of (i) a CD8 transmembrane domain polypeptide; (ii) a SLAMF7 transmembrane domain polypeptide; (iii) a CD86 transmembrane domain polypeptide; (iv) a DECTIN1 transmembrane domain polypeptide, and (v) a CSF1R transmembrane domain polypeptide; (d) a first intracellular signaling domain polypeptide, wherein the first intracellular signaling domain polypeptide is selected from the group consisting of: (i) a CD86 intracellular signaling domain polypeptide; (ii) a Lox1c intracellular signaling domain polypeptide, and (iii) a 2B4 intracellular signaling domain polypeptide, (iv) a DECTIN1 intracellular signaling domain polypeptide, and (v) a CD40 intracellular signaling domain polypeptide; and (e) a second intracellular domain polypeptide comprising a CD3ζ intracellular signaling domain polypeptide.

In some embodiments, provided herein is a CAR comprising: (a) an antigen recognition moiety polypeptide; (b) a CD8 hinge domain polypeptide; (c) a transmembrane domain polypeptide, wherein the transmembrane domain polypeptide is selected from the group consisting of (i) a CD8 transmembrane domain polypeptide; and (ii) a CSF1R transmembrane domain polypeptide; (d) and an intracellular domain polypeptide comprising a CD3ζ intracellular signaling domain polypeptide.

In certain embodiments, a CAR comprises a CD8 hinge domain polypeptide comprising an amino acid sequence at least 98% identical to SEQ ID NO:1 (e.g., 98%, 99%, or 100% identical to SEQ ID NO:1). In some embodiments, the CD8 hinge domain polypeptide comprises an amino acid sequence of SEQ ID NO:1.

In some embodiments, the CAR comprising a CD8 hinge domain polypeptide comprising an amino acid sequence at least 98% identical to SEQ ID NO:1 (e.g., 98%, 99%, or 100% identical to SEQ ID NO:1) further comprises a CD8 transmembrane domain polypeptide. In some embodiments, the CAR comprises a CD8 hinge domain polypeptide comprising an amino acid sequence of SEQ ID NO:1 and a CD8 transmembrane domain polypeptide. In some embodiments, the CAR comprises a CD8 hinge domain polypeptide comprising an amino acid sequence of SEQ ID NO:1 and a CD8 transmembrane domain polypeptide comprising an amino acid sequence of SEQ ID NO:11. In some embodiments, the CAR comprises a CD8 hinge domain polypeptide comprising an amino acid sequence of SEQ ID NO: 1 and a CD8 transmembrane domain polypeptide comprising an amino acid sequence of SEQ ID NO:11. In some embodiments, the CAR comprises a CD8 hinge domain polypeptide comprising an amino acid sequence of SEQ ID NO: 1 and a CD8 transmembrane domain polypeptide comprising an amino acid sequence of SEQ ID NO:11. In certain embodiments, the CAR comprising a CD8 hinge domain polypeptide and a CD8 transmembrane domain polypeptide further comprises a first intracellular signaling domain polypeptide comprising a CD86 intracellular signaling domain polypeptide. In some embodiments, the CAR comprises a CD86 intracellular signaling domain polypeptide comprising an amino acid sequence of SEQ ID NO:40. In some embodiments, the CAR comprises a CD8 hinge domain polypeptide comprising an amino acid sequence of SEQ ID NO: 1, a CD8 transmembrane domain polypeptide comprising an amino acid sequence of SEQ ID NO:11, and a CD86 intracellular signaling domain polypeptide comprising an amino acid sequence of SEQ ID NO:40. In still further embodiments, the CAR comprising a CD8 hinge domain polypeptide, a CD8 transmembrane domain polypeptide, and a first intracellular signaling domain polypeptide comprising a CD86 intracellular signaling domain polypeptide, further comprises a second intracellular signaling domain polypeptide comprising a CD3ζ intracellular signaling domain polypeptide. In some embodiments, the CAR comprises a CD8 hinge domain polypeptide comprising an amino acid sequence of SEQ ID NO: 1, a CD8 transmembrane domain polypeptide comprising an amino acid sequence of SEQ ID NO: 11, a CD86 intracellular signaling domain polypeptide comprising an amino acid sequence of SEQ ID NO:40, and a CD3ζ intracellular signaling domain polypeptide comprising an amino acid sequence of SEQ ID NO:89.

In some embodiments, the CAR comprising a CD8 hinge domain polypeptide comprising an amino acid sequence at least 98% identical to SEQ ID NO:1 (e.g., 98%, 99%, or 100% identical to SEQ ID NO:1) further comprises a CD8 transmembrane domain polypeptide. In some embodiments, the CAR comprises a CD8 hinge domain polypeptide comprising an amino acid sequence of SEQ ID NO:1 and a CD8 transmembrane domain polypeptide. In some embodiments, the CAR comprises a CD8 hinge domain polypeptide comprising an amino acid sequence of SEQ ID NO:1 and a CD8 transmembrane domain polypeptide comprising an amino acid sequence of SEQ ID NO: 11. In some embodiments, the CAR comprises a CD8 hinge domain polypeptide comprising an amino acid sequence of SEQ ID NO: 1 and a CD8 transmembrane domain polypeptide comprising an amino acid sequence of SEQ ID NO:11. In some embodiments, the CAR comprises a CD8 hinge domain polypeptide comprising an amino acid sequence of SEQ ID NO: 1 and a CD8 transmembrane domain polypeptide comprising an amino acid sequence of SEQ ID NO:11. In certain embodiments, the CAR comprising a CD8 hinge domain polypeptide and a CD8 transmembrane domain polypeptide further comprises a first intracellular signaling domain polypeptide comprising a LOX1C intracellular signaling domain polypeptide. In some embodiments, the CAR comprises a LOX1C intracellular signaling domain polypeptide comprising an amino acid sequence of SEQ ID NO:41. In some embodiments, the CAR comprises CD8 hinge domain polypeptide comprising an amino acid sequence of SEQ ID NO: 1, a CD8 transmembrane domain polypeptide comprising an amino acid sequence of SEQ ID NO: 11, and a LOX1C intracellular signaling domain polypeptide comprising an amino acid sequence of SEQ ID NO:41. In still further embodiments, the CAR comprising a CD8 hinge domain polypeptide, a CD8 transmembrane domain polypeptide, and a first intracellular signaling domain polypeptide comprising a LOX1C intracellular signaling domain polypeptide, further comprises a second intracellular signaling domain polypeptide comprising a CD3ζ intracellular signaling domain polypeptide. In some embodiments, the CAR comprises a CD8 hinge domain polypeptide comprising an amino acid sequence of SEQ ID NO: 1, a CD8 transmembrane domain polypeptide comprising an amino acid sequence of SEQ ID NO: 11, a LOX1C intracellular signaling domain polypeptide comprising an amino acid sequence of SEQ ID NO:41, and a CD3ζ intracellular signaling domain polypeptide comprising an amino acid sequence of SEQ ID NO:89.

In some embodiments, the CAR comprising a CD8 hinge domain polypeptide comprising an amino acid sequence at least 98% identical to SEQ ID NO:1 (e.g., 98%, 99%, or 100% identical to SEQ ID NO:1) further comprises a CD8 transmembrane domain polypeptide. In some embodiments, the CAR comprises a CD8 hinge domain polypeptide comprising an amino acid sequence of SEQ ID NO:1 and a CD8 transmembrane domain polypeptide. In some embodiments, the CAR comprises a CD8 hinge domain polypeptide comprising an amino acid sequence of SEQ ID NO:1 and a CD8 transmembrane domain polypeptide comprising an amino acid sequence of SEQ ID NO:11. In some embodiments, the CAR comprises a CD8 hinge domain polypeptide comprising an amino acid sequence of SEQ ID NO:1 and a CD8 transmembrane domain polypeptide comprising an amino acid sequence of SEQ ID NO: 11. In some embodiments, the CAR comprises a CD8 hinge domain polypeptide comprising an amino acid sequence of SEQ ID NO:1 and a CD8 transmembrane domain polypeptide comprising an amino acid sequence of SEQ ID NO:11. In certain embodiments, the CAR comprising a CD8 hinge domain polypeptide and a CD8 transmembrane domain polypeptide further comprises an intracellular signaling domain polypeptide comprising a CD3ζ intracellular signaling domain polypeptide. In some embodiments, the CAR comprises a CD8 hinge domain polypeptide comprising an amino acid sequence of SEQ ID NO: 1, a CD8 transmembrane domain polypeptide comprising an amino acid sequence of SEQ ID NO:11, and a CD3ζ intracellular signaling domain polypeptide comprising an amino acid sequence of SEQ ID NO:89.

In some embodiments, the CAR comprising a CD8 hinge domain polypeptide comprising an amino acid sequence at least 98% identical to SEQ ID NO:1 (e.g., 98%, 99%, or 100% identical to SEQ ID NO:1) further comprises a SLAMF7 transmembrane domain polypeptide. In some embodiments, the CAR comprises a CD8 hinge domain polypeptide comprising an amino acid sequence of SEQ ID NO: 1 and a SLAMF7 transmembrane domain polypeptide. In some embodiments, the CAR comprises a CD8 hinge domain polypeptide comprising an amino acid sequence of SEQ ID NO:1 and a SLAMF7 transmembrane domain polypeptide comprising an amino acid sequence of SEQ ID NO:12. In certain embodiments, the CAR comprising a CD8 hinge domain polypeptide and a SLAMF7 transmembrane domain polypeptide further comprises a first intracellular signaling domain polypeptide comprising a CD86 intracellular signaling domain polypeptide. In some embodiments, the CAR comprises a CD86 intracellular signaling domain polypeptide comprising an amino acid sequence of SEQ ID NO: 40. In some embodiments, the CAR comprises a CD8 hinge domain polypeptide comprising an amino acid sequence of SEQ ID NO: 1, a SLAMF7 transmembrane domain polypeptide comprising an amino acid sequence of SEQ ID NO:12, and a CD86 intracellular signaling domain polypeptide comprising an amino acid sequence of SEQ ID NO:40. In still further embodiments, the CAR comprising a CD8 hinge domain polypeptide, a SLAMF7 transmembrane domain polypeptide, and a first intracellular signaling domain polypeptide comprising a CD86 intracellular signaling domain polypeptide, further comprises a second intracellular signaling domain polypeptide comprising a CD3ζ intracellular signaling domain polypeptide. In some embodiments, the CAR comprises a CD8 hinge domain polypeptide comprising an amino acid sequence of SEQ ID NO: 1, a SLAMF7 transmembrane domain polypeptide comprising an amino acid sequence of SEQ ID NO:12, a CD86 intracellular signaling domain polypeptide comprising an amino acid sequence of SEQ ID NO:40, and a CD3ζ intracellular signaling domain polypeptide comprising an amino acid sequence of SEQ ID NO: 89.

In certain embodiments, a CAR comprises a CD8 hinge domain polypeptide comprising an amino acid sequence at least 98% identical to SEQ ID NO:1 (e.g., 98%, 99%, or 100% identical to SEQ ID NO:1). In some embodiments, the CAR comprising a CD8 hinge domain polypeptide comprising an amino acid sequence at least 98% identical to SEQ ID NO:1 (e.g., 98%, 99%, or 100% identical to SEQ ID NO:1) further comprises a CD8 transmembrane domain polypeptide. In some embodiments, the CAR comprises a CD8 hinge domain polypeptide comprising an amino acid sequence of SEQ ID NO:1 and a CD8 transmembrane domain polypeptide. In certain embodiments, the CAR comprising a CD8 hinge domain polypeptide and a CD8 transmembrane domain polypeptide further comprises a first intracellular signaling domain polypeptide comprising a CD40 intracellular signaling domain polypeptide. In some embodiments, the CAR comprises a CD40 intracellular signaling domain polypeptide comprising an amino acid sequence of SEQ ID NO:46. In still further embodiments, the CAR comprising a CD8 hinge domain polypeptide, a CD8 transmembrane domain polypeptide, and a first intracellular signaling domain polypeptide comprising a CD40 intracellular signaling domain polypeptide, further comprises a second intracellular signaling domain polypeptide comprising a CD3ζ intracellular signaling domain polypeptide. In some embodiments, the CAR comprises a CD8 hinge domain polypeptide comprising an amino acid sequence of SEQ ID NO: 1, a CD8 transmembrane domain polypeptide comprising an amino acid sequence of SEQ ID NO: 11, a CD40 intracellular signaling domain polypeptide comprising an amino acid sequence of SEQ ID NO:46, and a CD3ζ intracellular signaling domain polypeptide comprising an amino acid sequence of SEQ ID NO:89.

In some embodiments, the CAR comprising a CD8 hinge domain polypeptide comprising an amino acid sequence at least 98% identical to SEQ ID NO:1 (e.g., 98%, 99%, or 100% identical to SEQ ID NO:1) further comprises a CD86 transmembrane domain polypeptide. In some embodiments, the CAR comprises a CD8 hinge domain polypeptide comprising an amino acid sequence of SEQ ID NO:1 and a CD86 transmembrane domain polypeptide. In some embodiments, the CAR comprises a CD8 hinge domain polypeptide comprising an amino acid sequence of SEQ ID NO: 1 and a CD86 transmembrane domain polypeptide comprising an amino acid sequence of SEQ ID NO:13. In some embodiments, the CAR comprises a CD8 hinge domain polypeptide comprising an amino acid sequence of SEQ ID NO: 1 and a CD86 transmembrane domain polypeptide comprising an amino acid sequence of SEQ ID NO:13. In some embodiments, the CAR comprises a CD8 hinge domain polypeptide comprising an amino acid sequence of SEQ ID NO: 1 and a CD86 transmembrane domain polypeptide comprising an amino acid sequence of SEQ ID NO:13. In certain embodiments, the CAR comprising a CD8 hinge domain polypeptide and a CD86 transmembrane domain polypeptide further comprises a first intracellular signaling domain polypeptide comprising a 2B4 intracellular signaling domain polypeptide. In some embodiments, the CAR comprises a 2B4 intracellular signaling domain polypeptide comprising an amino acid sequence of SEQ ID NO:42. In some embodiments, the CAR comprises a CD8 hinge domain polypeptide comprising an amino acid sequence of SEQ ID NO: 1, a CD86 transmembrane domain polypeptide comprising an amino acid sequence of SEQ ID NO: 13, and a 2B4 intracellular signaling domain polypeptide comprising an amino acid sequence of SEQ ID NO:42. In still further embodiments, the CAR comprising a CD8 hinge domain polypeptide, a CD86 transmembrane domain polypeptide, and a first intracellular signaling domain polypeptide comprising a 2B4 intracellular signaling domain polypeptide, further comprises a second intracellular signaling domain polypeptide comprising a CD3ζ intracellular signaling domain polypeptide. In some embodiments, the CAR comprises a CD8 hinge domain polypeptide comprising an amino acid sequence of SEQ ID NO: 1, a CD86 transmembrane domain polypeptide comprising an amino acid sequence of SEQ ID NO:13, a 2B4 intracellular signaling domain polypeptide comprising an amino acid sequence of SEQ ID NO: 42, and a CD3ζ intracellular signaling domain polypeptide comprising an amino acid sequence of SEQ ID NO:89.

In some embodiments, the CAR comprising a CD8 hinge domain polypeptide comprising an amino acid sequence at least 98% identical to SEQ ID NO:1 (e.g., 98%, 99%, or 100% identical to SEQ ID NO:1) further comprises a DECTIN1 transmembrane domain polypeptide. In some embodiments, the CAR comprises a CD8 hinge domain polypeptide comprising an amino acid sequence of SEQ ID NO: 1 and a DECTIN1 transmembrane domain polypeptide. In some embodiments, the CAR comprises a CD8 hinge domain polypeptide comprising an amino acid sequence of SEQ ID NO: 1 and a DECTIN1 transmembrane domain polypeptide comprising an amino acid sequence of SEQ ID NO:14. In some embodiments, the CAR comprises a CD8 hinge domain polypeptide comprising an amino acid sequence of SEQ ID NO: 1 and a DECTIN1 transmembrane domain polypeptide comprising an amino acid sequence of SEQ ID NO: 14. In some embodiments, the CAR comprises a CD8 hinge domain polypeptide comprising an amino acid sequence of SEQ ID NO: 1 and a DECTIN1 transmembrane domain polypeptide comprising an amino acid sequence of SEQ ID NO:14. In certain embodiments, the CAR comprising a CD8 hinge domain polypeptide and a DECTIN1 transmembrane domain polypeptide further comprises a first intracellular signaling domain polypeptide comprising a DECTIN1 intracellular signaling domain polypeptide. In some embodiments, the CAR comprises a DECTIN1 intracellular signaling domain polypeptide comprising an amino acid sequence of SEQ ID NO:43. In some embodiments, the CAR comprises a CD8 hinge domain polypeptide comprising an amino acid sequence of SEQ ID NO: 1, a DECTIN1 transmembrane domain polypeptide comprising an amino acid sequence of SEQ ID NO: 14, and a DECTIN1 intracellular signaling domain polypeptide comprising an amino acid sequence of SEQ ID NO: 43. In still further embodiments, the CAR comprising a CD8 hinge domain polypeptide, a DECTIN1 transmembrane domain polypeptide, and a first intracellular signaling domain polypeptide comprising a DECTIN1 intracellular signaling domain polypeptide, further comprises a second intracellular signaling domain polypeptide comprising a CD3ζ intracellular signaling domain polypeptide. In some embodiments, the CAR comprises a CD8 hinge domain polypeptide comprising an amino acid sequence of SEQ ID NO: 1, a DECTIN1 transmembrane domain polypeptide comprising an amino acid sequence of SEQ ID NO: 14, a DECTIN1 intracellular signaling domain polypeptide comprising an amino acid sequence of SEQ ID NO: 43, and a CD3ζ intracellular signaling domain polypeptide comprising an amino acid sequence of SEQ ID NO:89.

In some embodiments, the CAR comprising a CD8 hinge domain polypeptide comprising an amino acid sequence at least 98% identical to SEQ ID NO:1 (e.g., 98%, 99%, or 100% identical to SEQ ID NO:1) further comprises a CSF1R transmembrane domain polypeptide. In some embodiments, the CAR comprises a CD8 hinge domain polypeptide comprising an amino acid sequence of SEQ ID NO: 1 and a CSF1R transmembrane domain polypeptide. In some embodiments, the CAR comprises a CD8 hinge domain polypeptide comprising an amino acid sequence of SEQ ID NO: 1 and a CSF1R transmembrane domain polypeptide comprising an amino acid sequence of SEQ ID NO:33. In certain embodiments, the CAR comprising a CD8 hinge domain polypeptide and a CSF1R transmembrane domain polypeptide further comprises an intracellular signaling domain polypeptide comprising a CD3ζ intracellular signaling domain polypeptide. In some embodiments, the CAR comprises a CD8 hinge domain polypeptide comprising an amino acid sequence of SEQ ID NO: 1, a CSF1R transmembrane domain polypeptide comprising an amino acid sequence of SEQ ID NO:33, and a CD3ζ intracellular signaling domain polypeptide comprising an amino acid sequence of SEQ ID NO: 89.

In certain embodiments, a CAR comprises a CD8 hinge domain polypeptide comprising an amino acid sequence at least 98% identical to SEQ ID NO:4 (e.g., 98%, 99%, or 100% identical to SEQ ID NO:4). In some embodiments, the CAR comprising a CD8 hinge domain polypeptide comprising an amino acid sequence at least 98% identical to SEQ ID NO:4 (e.g., 98%, 99%, or 100% identical to SEQ ID NO:1) further comprises a SLAMF7 transmembrane domain polypeptide. In some embodiments, the CAR comprises a CD8 hinge domain polypeptide comprising an amino acid sequence of SEQ ID NO:4 and a SLAMF7 transmembrane domain polypeptide. In some embodiments, the CAR comprises a CD8 hinge domain polypeptide comprising an amino acid sequence of SEQ ID NO:4 and a SLAMF7 transmembrane domain polypeptide comprising an amino acid sequence of SEQ ID NO: 12. In certain embodiments, the CAR comprising a CD8 hinge domain polypeptide and a SLAMF7 transmembrane domain polypeptide further comprises a first intracellular signaling domain polypeptide comprising a CD86 intracellular signaling domain polypeptide. In some embodiments, CAR comprises a CD86 intracellular signaling domain polypeptide comprising an amino acid sequence of SEQ ID NO:40. In some embodiments, CAR comprises a CD8 hinge domain polypeptide comprising an amino acid sequence of SEQ ID NO: 4, a SLAMF7 transmembrane domain polypeptide comprising an amino acid sequence of SEQ ID NO:12, and a CD86 intracellular signaling domain polypeptide comprising an amino acid sequence of SEQ ID NO: 40. In still further embodiments, the CAR comprising a CD8 hinge domain polypeptide, a SLAMF7 transmembrane domain polypeptide, and a first intracellular signaling domain polypeptide comprising a CD86 intracellular signaling domain polypeptide, further comprises a second intracellular signaling domain polypeptide comprising a CD3ζ intracellular signaling domain polypeptide. In some embodiments, CAR comprises a CD8 hinge domain polypeptide comprising an amino acid sequence of SEQ ID NO: 4, a SLAMF7 transmembrane domain polypeptide comprising an amino acid sequence of SEQ ID NO:12, a CD86 intracellular signaling domain polypeptide comprising an amino acid sequence of SEQ ID NO:40, and a CD3ζ intracellular signaling domain polypeptide comprising an amino acid sequence of SEQ ID NO: 89.

In certain embodiments, a CAR comprises a CD8 hinge domain polypeptide comprising an amino acid sequence at least 98% identical to SEQ ID NO:4 (e.g., 98%, 99%, or 100% identical to SEQ ID NO:4). In some embodiments, the CAR comprising a CD8 hinge domain polypeptide comprising an amino acid sequence at least 98% identical to SEQ ID NO:4 (e.g., 98%, 99%, or 100% identical to SEQ ID NO:1) further comprises a CD8 transmembrane domain polypeptide. In some embodiments, the CAR comprises a CD8 hinge domain polypeptide comprising an amino acid sequence of SEQ ID NO:4 and a CD8 transmembrane domain polypeptide. In some embodiments, the CAR comprises a CD8 hinge domain polypeptide comprising an amino acid sequence of SEQ ID NO:4 and a CD8 transmembrane domain polypeptide comprising an amino acid sequence of SEQ ID NO:11. In certain embodiments, the CAR comprising a CD8 hinge domain polypeptide and a CD8 transmembrane domain polypeptide further comprises a first intracellular signaling domain polypeptide comprising a CD86 intracellular signaling domain polypeptide. In some embodiments, the CAR comprises a CD86 intracellular signaling domain polypeptide comprising an amino acid sequence of SEQ ID NO:40. In some embodiments, a CAR comprises a CD8 hinge domain polypeptide comprising an amino acid sequence of SEQ ID NO: 4, a SLAMF7 transmembrane domain polypeptide comprising an amino acid sequence of SEQ ID NO:11, and a CD86 intracellular signaling domain polypeptide comprising an amino acid sequence of SEQ ID NO: 40. In still further embodiments, the CAR comprising a CD8 hinge domain polypeptide, a CD8 transmembrane domain polypeptide, and a first intracellular signaling domain polypeptide comprising a CD86 intracellular signaling domain polypeptide, further comprises a second intracellular signaling domain polypeptide comprising a CD3ζ intracellular signaling domain polypeptide. In some embodiments, the CAR comprises a CD8 hinge domain polypeptide comprising an amino acid sequence of SEQ ID NO: 4, a CD8 transmembrane domain polypeptide comprising an amino acid sequence of SEQ ID NO:11, a CD86 intracellular signaling domain polypeptide comprising an amino acid sequence of SEQ ID NO:40, and a CD3ζ intracellular signaling domain polypeptide comprising an amino acid sequence of SEQ ID NO: 89.

In some embodiments, the CAR comprising a CD8 hinge domain polypeptide comprising an amino acid sequence at least 98% identical to SEQ ID NO:4 (e.g., 98%, 99%, or 100% identical to SEQ ID NO:4) further comprises a CD8 transmembrane domain polypeptide. In some embodiments, the CAR comprises a CD8 hinge domain polypeptide comprising an amino acid sequence of SEQ ID NO:4 and a CD8 transmembrane domain polypeptide. In some embodiments, the CAR comprises a CD8 hinge domain polypeptide comprising an amino acid sequence of SEQ ID NO:4 and a CD8 transmembrane domain polypeptide comprising an amino acid sequence of SEQ ID NO: 11. In some embodiments, the CAR comprises a CD8 hinge domain polypeptide comprising an amino acid sequence of SEQ ID NO:4 and a CD8 transmembrane domain polypeptide comprising an amino acid sequence of SEQ ID NO: 11. In some embodiments, the CAR comprises a CD8 hinge domain polypeptide comprising an amino acid sequence of SEQ ID NO:4 and a CD8 transmembrane domain polypeptide comprising an amino acid sequence of SEQ ID NO:11. In certain embodiments, the CAR comprising a CD8 hinge domain polypeptide and a CD8 transmembrane domain polypeptide further comprises an intracellular signaling domain polypeptide comprising a CD3ζ intracellular signaling domain polypeptide. In some embodiments, the CAR comprises a CD8 hinge domain polypeptide comprising an amino acid sequence of SEQ ID NO: 4, a CD8 transmembrane domain polypeptide comprising an amino acid sequence of SEQ ID NO:11, and a CD3ζ intracellular signaling domain polypeptide comprising an amino acid sequence of SEQ ID NO:89.

In certain embodiments, a CAR comprises a CD8 hinge domain polypeptide comprising an amino acid sequence at least 98% identical to SEQ ID NO:4 (e.g., 98%, 99%, or 100% identical to SEQ ID NO:4). In some embodiments, the CAR comprising a CD8 hinge domain polypeptide comprising an amino acid sequence at least 98% identical to SEQ ID NO:4 (e.g., 98%, 99%, or 100% identical to SEQ ID NO:1) further comprises a CD8 transmembrane domain polypeptide. In some embodiments, the CAR comprises a CD8 hinge domain polypeptide comprising an amino acid sequence of SEQ ID NO:4 and a CD8 transmembrane domain polypeptide. In certain embodiments, the CAR comprising a CD8 hinge domain polypeptide and a CD8 transmembrane domain polypeptide further comprises a first intracellular signaling domain polypeptide comprising a CD40 intracellular signaling domain polypeptide. In some embodiments, the CAR comprises a CD40 intracellular signaling domain polypeptide comprising an amino acid sequence of SEQ ID NO:46. In still further embodiments, the CAR comprising a CD8 hinge domain polypeptide, a CD8 transmembrane domain polypeptide, and a first intracellular signaling domain polypeptide comprising a CD40 intracellular signaling domain polypeptide, further comprises a second intracellular signaling domain polypeptide comprising a CD3, intracellular signaling domain polypeptide. In some embodiments, the CAR comprises a CD8 hinge domain polypeptide comprising an amino acid sequence of SEQ ID NO: 4, a CD8 transmembrane domain polypeptide comprising an amino acid sequence of SEQ ID NO: 11, a CD40 intracellular signaling domain polypeptide comprising an amino acid sequence of SEQ ID NO:46, and a CD3ζ intracellular signaling domain polypeptide comprising an amino acid sequence of SEQ ID NO:89.

In some embodiments, the CAR provided herein comprises an antigen recognition moiety polypeptide that comprises a scFv antigen recognition moiety polypeptide. In some embodiments, the scFv antigen recognition moiety polypeptide recognizes a tumor antigen or fragment thereof. In specific embodiments, the tumor antigen is selected from your group consisting of HER2/neu, PSMA, Claudin18, CD20, CD5, BCMA, mesothelin, TAC1, GD2, and CD19. In some embodiments, the tumor antigen comprises HER2/neu. In some embodiments, the tumor antigen comprises PSMA. In some embodiments, the tumor antigen comprises Claudin18. In some embodiments, the tumor antigen comprises CD20. In some embodiments, the tumor antigen comprises CD5. In some embodiments, the tumor antigen comprises BCMA. In some embodiments, the tumor antigen comprises mesothelin. In some embodiments, the tumor antigen comprises TAC1. In some embodiments, the tumor antigen comprises GD2. In some embodiments, the tumor antigen comprises CD19.

In some aspects, the chimeric antigen receptor (CAR) of the present disclosure comprises a signal peptide. In some embodiments, the signal peptide has an amino acid sequence of MALPVTALLLPLALLLHAARP (SEQ ID NO: 2). In some embodiments, the signal peptide has an amino acid sequence with at least 80% sequence identity with SEQ ID NO: 2. In some embodiments, the signal peptide has an amino acid sequence with at least 90%, 95%, 97% or 99% sequence identity with SEQ ID NO: 2. In some embodiments, the signal peptide has an amino acid sequence of METDTLLLWVLLLWVPGSTG (SEQ ID NO: 109). In some embodiments, the signal peptide has an amino acid sequence with at least 80% sequence identity with SEQ ID NO: 109. In some embodiments, the signal peptide has an amino acid sequence with at least 90%, 95%, 97% or 99% sequence identity with SEQ ID NO: 109.

In specific embodiments, a CAR provided herein comprises an amino acid sequence at least 90%, at least 95%, or 100% identical to an amino acid sequence provided in Table 7. In some embodiments, a CAR provided herein comprises an amino acid sequence at least 90%, at least 95%, or 100% identical to SEQ ID NO:97-108. In some embodiments, a CAR provided herein comprises an amino acid sequence at least 90%, at least 95%, or 100% identical to SEQ ID NO: 97. In some embodiments, a CAR provided herein comprises an amino acid sequence at least 90%, at least 95%, or 100% identical to SEQ ID NO:98. In some embodiments, a CAR provided herein comprises an amino acid sequence at least 90%, at least 95%, or 100% identical to SEQ ID NO:99. In some embodiments, a CAR provided herein comprises an amino acid sequence at least 90%, at least 95%, or 100% identical to SEQ ID NO: 100. In some embodiments, a CAR provided herein comprises an amino acid sequence at least 90%, at least 95%, or 100% identical to SEQ ID NO:101. In some embodiments, a CAR provided herein comprises an amino acid sequence at least 90%, at least 95%, or 100% identical to SEQ ID NO: 102. In some embodiments, a CAR provided herein comprises an amino acid sequence at least 90%, at least 95%, or 100% identical to SEQ ID NO:103. In some embodiments, a CAR provided herein comprises an amino acid sequence at least 90%, at least 95%, or 100% identical to SEQ ID NO:104. In some embodiments, a CAR provided herein comprises an amino acid sequence at least 90%, at least 95%, or 100% identical to SEQ ID NO: 105. In some embodiments, a CAR provided herein comprises an amino acid sequence at least 90%, at least 95%, or 100% identical to SEQ ID NO:106. In some embodiments, a CAR provided herein comprises an amino acid sequence at least 90%, at least 95%, or 100% identical to SEQ ID NO: 107. In some embodiments, a CAR provided herein comprises an amino acid sequence at least 90%, at least 95%, or 100% identical to SEQ ID NO:108.

In some embodiments, a CAR provided herein comprises an amino acid sequence at least 90%, at least 95%, or 100% identical to SEQ ID NO:97-108, without the signal peptide. In some embodiments, a CAR provided herein comprises an amino acid sequence at least 90%, at least 95%, or 100% identical to SEQ ID NO:97-108, with an alternative signal peptide. In a specific embodiment, the alternative signal peptide is SEQ ID NO: 109.

In some embodiments, a CAR provided herein comprises an amino acid sequence at least 90%, at least 95%, or 100% identical to SEQ ID NO:110-121. In some embodiments, a CAR provided herein comprises an amino acid sequence at least 90%, at least 95%, or 100% identical to SEQ ID NO:110. In some embodiments, a CAR provided herein comprises an amino acid sequence at least 90%, at least 95%, or 100% identical to SEQ ID NO:111. In some embodiments, a CAR provided herein comprises an amino acid sequence at least 90%, at least 95%, or 100% identical to SEQ ID NO:112. In some embodiments, a CAR provided herein comprises an amino acid sequence at least 90%, at least 95%, or 100% identical to SEQ ID NO: 113. In some embodiments, a CAR provided herein comprises an amino acid sequence at least 90%, at least 95%, or 100% identical to SEQ ID NO:114. In some embodiments, a CAR provided herein comprises an amino acid sequence at least 90%, at least 95%, or 100% identical to SEQ ID NO:115. In some embodiments, a CAR provided herein comprises an amino acid sequence at least 90%, at least 95%, or 100% identical to SEQ ID NO:116. In some embodiments, a CAR provided herein comprises an amino acid sequence at least 90%, at least 95%, or 100% identical to SEQ ID NO:117. In some embodiments, a CAR provided herein comprises an amino acid sequence at least 90%, at least 95%, or 100% identical to SEQ ID NO: 118. In some embodiments, a CAR provided herein comprises an amino acid sequence at least 90%, at least 95%, or 100% identical to SEQ ID NO:119. In some embodiments, a CAR provided herein comprises an amino acid sequence at least 90%, at least 95%, or 100% identical to SEQ ID NO: 120. In some embodiments, a CAR provided herein comprises an amino acid sequence at least 90%, at least 95%, or 100% identical to SEQ ID NO:121. In some embodiments, a CAR provided herein comprises an amino acid sequence at least 90%, at least 95%, or 100% identical to SEQ ID NO:125. In some embodiments, a CAR provided herein comprises an amino acid sequence at least 90%, at least 95%, or 100% identical to SEQ ID NO: 126. In some embodiments, a CAR provided herein comprises an amino acid sequence at least 90%, at least 95%, or 100% identical to SEQ ID NO:127. In some embodiments, a CAR provided herein comprises an amino acid sequence at least 90%, at least 95%, or 100% identical to SEQ ID NO:128. In some embodiments, a CAR provided herein comprises an amino acid sequence at least 90%, at least 95%, or 100% identical to SEQ ID NO: 129. In some embodiments, a CAR provided herein comprises an amino acid sequence at least 90%, at least 95%, or 100% identical to SEQ ID NO:130. In some embodiments, a CAR provided herein comprises an amino acid sequence at least 90%, at least 95%, or 100% identical to SEQ ID NO: 131. In some embodiments, a CAR provided herein comprises an amino acid sequence at least 90%, at least 95%, or 100% identical to SEQ ID NO:132.

TABLE 7
Polypeptide sequences encoding exemplary CARs

| | | SEQ |
|---|---|---|
| CAR | Sequence | ID NO |
| 1 | MALPVTALLLPLALLLHAARPDIQMTQTTSSLSASLGDRVTISCRASQDISKYLNWYQ | 97 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDIYIWAPLAGTCGVLLLSLVITKWKKKKRPRNSYKCGTNTM | |
| | EREESEQTKKREKIHIPERSDEAQRVFKSSKTSSCDKSDTCFRVKFSRSADAPAYQQG | |
| | QNQLYNELNLGRREEYDVLDKRRGRDPEMGGKPQRRKNPQEGLYNELQKDKMAEAYSE | |
| | IGMKGERRRGKGHDGLYQGLSTATKDTYDALHMQALPPR | |
| 2 | MALPVTALLLPLALLLHAARPDIQMTQTTSSLSASLGDRVTISCRASQDISKYLNWYQ | 98 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDIYIWAPLAGTCGVLLLSLVITKWKKKKRPRNSYKCGTNTM | |
| | EREESEQTKKREKIHIPERSDEAQRVFKSSKTSSCDKSDTCFRVKFSRSADAPAYQQG | |
| | QNQLYNELNLGRREEYDVLDKRRGRDPEMGGKPQRRKNPQEGLYNELQKDKMAEAYSE | |
| | IGMKGERRRGKGHDGLYQGLSTATKDTYDALHMQALPPR | |
| 3 | MALPVTALLLPLALLLHAARPDIQMTQTTSSLSASLGDRVTISCRASQDISKYLNWYQ | 99 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDIYIWAPLAGTCGVLLLSLVITCWWPSYLFQLGKAKKGNSK | |
| | EDPQDKVTQIKLDDFTMRVKFSRSADAPAYQQGQNQLYNELNLGRREEYDVLDKRRGR | |
| | DPEMGGKPQRRKNPQEGLYNELQKDKMAEAYSEIGMKGERRRGKGHDGLYQGLSTATK | |
| | DTYDALHMQALPPR | |
| 4 | MALPVTALLLPLALLLHAARPDIQMTQTTSSLSASLGDRVTISCRASQDISKYLNWYQ | 100 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDVLLCLLLVPLLLSLFVLGLFLKWKKKKRPRNSYKCGTNTM | |
| | EREESEQTKKREKIHIPERSDEAQRVFKSSKTSSCDKSDTCFRVKFSRSADAPAYQQG | |
| | QNQLYNELNLGRREEYDVLDKRRGRDPEMGGKPQRRKNPQEGLYNELQKDKMAEAYSE | |
| | IGMKGERRRGKGHDGLYQGLSTATKDTYDALHMQALPPR | |
| 5 | MALPVTALLLPLALLLHAARPDIQMTQTTSSLSASLGDRVTISCRASQDISKYLNWYQ | 101 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDWITAVLPTVIICVMVFCLILWWRRKRKEKQSETSPKEFLT | |
| | IYEDVKDLKTRRNHEQEQTFPGGGSTIYSMIQSQSSAPTSTGGGSGGSRVKFSRSADA | |
| | PAYQQGQNQLYNELNLGRREEYDVLDKRRGRDPEMGGKPQRRKNPQEGLYNELQKDKM | |
| | AEAYSEIGMKGERRRGKGHDGLYQGLSTATKDTYDALHMQALPPR | |
| 6 | MALPVTALLLPLALLLHAARPDIQMTQTTSSLSASLGDRVTISCRASQDISKYLNWYQ | 102 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDGLVVAIVLIVLCLIGLIVAILRWPPSAACSGKESVVAIRT | |
| | NSQSDFHLQTYGDEDLNELDPHYEMRVKFSRSADAPAYQQGQNQLYNELNLGRREEYD | |
| | VLDKRRGRDPEMGGKPQRRKNPQEGLYNELQKDKMAEAYSEIGMKGERRRGKGHDGLY | |
| | QGLSTATKDTYDALHMQALPPR | |
| 7 | MALPVTALLLPLALLLHAARPDIQMTQTTSSLSASLGDRVTISCRASQDISKYLNWYQ | 103 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSALSNSIMYFSHFVPVFLPAKPTTTPAP | |
| | RPPTPAPTIASQPLSLRPEACRPAAGGAVHTRGLDFACDIYIWAPLAGTCGVLLLSLV | |
| | ITRVKFSRSADAPAYQQGQNQLYNELNLGRREEYDVLDKRRGRDPEMGGKPQRRKNPQ | |
| | EGLYNELQKDKMAEAYSEIGMKGERRRGKGHDGLYQGLSTATKDTYDALHMQALPPR | |
| 8 | MALPVTALLLPLALLLHAARPDIQMTQTTSSLSASLGDRVTISCRASQDISKYLNWYQ | 104 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSALSNSIMYFSHFVPVFLPAKPTTTPAP | |
| | RPPTPAPTIASQPLSLRPEACRPAAGGAVHTRGLDFACDIYIWAPLAGTCGVLLLSLV | |
| | ITKWKKKKRPRNSYKCGTNTMEREESEQTKKREKIHIPERSDEAQRVFKSSKTSSCDK | |
| | SDTCFRVKFSRSADAPAYQQGQNQLYNELNLGRREEYDVLDKRRGRDPEMGGKPQRRK | |
| | NPQEGLYNELQKDKMAEAYSEIGMKGERRRGKGHDGLYQGLSTATKDTYDALHMQALP | |
| | PR | |
| 9 | MALPVTALLLPLALLLHAARPDIQMTQTTSSLSASLGDRVTISCRASQDISKYLNWYQ | 105 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSALSNSIMYFSHFVPVFLPAKPTTTPAP | |
| | RPPTPAPTIASQPLSLRPEACRPAAGGAVHTRGLDFACDVLLCLLLVPLLLSLFVLGL | |
| | FLKWKKKKRPRNSYKCGTNTMEREESEQTKKREKIHIPERSDEAQRVFKSSKTSSCDK | |
| | SDTCFRVKFSRSADAPAYQQGQNQLYNELNLGRREEYDVLDKRRGRDPEMGGKPQRRK | |
| | NPQEGLYNELQKDKMAEAYSEIGMKGERRRGKGHDGLYQGLSTATKDTYDALHMQALP | |
| | PR | |
| 10 | MALPVTALLLPLALLLHAARPDIQMTQTTSSLSASLGDRVTISCRASQDISKYLNWYQ | 106 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDIYIWAPLAGTCGVLLLSLVITTYCFAPRCRERRRNERLRR | |
| | ESVRPVRVKFSRSADAPAYQQGQNQLYNELNLGRREEYDVLDKRRGRDPEMGGKPQRR | |
| | KNPQEGLYNELQKDKMAEAYSEIGMKGERRRGKGHDGLYQGLSTATKDTYDALHMQAL | |
| | PPR | |
| 11 | MALPVTALLLPLALLLHAARPDIQMTQTTSSLSASLGDRVTISCRASQDISKYLNWYQ | 107 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDVLLCLLLVPLLLSLFVLGLFLTTYCFAPRCRERRRNERLR | |
| | RESVRPVRVKFSRSADAPAYQQGQNQLYNELNLGRREEYDVLDKRRGRDPEMGGKPQR | |
| | RKNPQEGLYNELQKDKMAEAYSEIGMKGERRRGKGHDGLYQGLSTATKDTYDALHMQA | |
| | LPPR | |
| 12 | MALPVTALLLPLALLLHAARPDIQMTQTTSSLSASLGDRVTISCRASQDISKYLNWYQ | 108 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDIYIWAPLAGTCGVLLLSLVITTYCFAPRCRERRRNERLRR | |
| | ESVRPVRVKFSRSADAPAYQQGQNQLYNELNLGRREEYDVLDKRRGRDPEMGGKPQRR | |
| | KNPQEGLYNELQKDKMAEAYSEIGMKGERRRGKGHDGLYQGLSTATKDTYDALHMQAL | |
| | PPR | |
| 13 | DIQMTQTTSSLSASLGDRVTISCRASQDISKYLNWYQQKPDGTVKLLIYHTSRLHSGV | 110 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFACDIYI | |
| | WAPLAGTCGVLLLSLVITKWKKKKRPRNSYKCGTNTMEREESEQTKKREKIHIPERSD | |
| | EAQRVFKSSKTSSCDKSDTCFRVKFSRSADAPAYQQGQNQLYNELNLGRREEYDVLDK | |
| | RRGRDPEMGGKPQRRKNPQEGLYNELQKDKMAEAYSEIGMKGERRRGKGHDGLYQGLS | |
| | TATKDTYDALHMQALPPR | |
| 14 | DIQMTQTTSSLSASLGDRVTISCRASQDISKYLNWYQQKPDGTVKLLIYHTSRLHSGV | 111 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFACDIYI | |
| | WAPLAGTCGVLLLSLVITKWKKKKRPRNSYKCGTNTMEREESEQTKKREKIHIPERSD | |
| | EAQRVFKSSKTSSCDKSDTCFRVKFSRSADAPAYQQGQNQLYNELNLGRREEYDVLDK | |
| | RRGRDPEMGGKPQRRKNPQEGLYNELQKDKMAEAYSEIGMKGERRRGKGHDGLYQGLS | |
| | TATKDTYDALHMQALPPR | |
| 15 | DIQMTQTTSSLSASLGDRVTISCRASQDISKYLNWYQQKPDGTVKLLIYHTSRLHSGV | 112 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFACDIYI | |
| | WAPLAGTCGVLLLSLVITCWWPSYLFQLGKAKKGNSKEDPQDKVTQIKLDDFTMRVKF | |
| | SRSADAPAYQQGQNQLYNELNLGRREEYDVLDKRRGRDPEMGGKPQRRKNPQEGLYNE | |
| | LQKDKMAEAYSEIGMKGERRRGKGHDGLYQGLSTATKDTYDALHMQALPPR | |
| 16 | DIQMTQTTSSLSASLGDRVTISCRASQDISKYLNWYQQKPDGTVKLLIYHTSRLHSGV | 113 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFACDVLL | |
| | CLLLVPLLLSLFVLGLFLKWKKKKRPRNSYKCGTNTMEREESEQTKKREKIHIPERSD | |
| | EAQRVFKSSKTSSCDKSDTCFRVKFSRSADAPAYQQGQNQLYNELNLGRREEYDVLDK | |
| | RRGRDPEMGGKPQRRKNPQEGLYNELQKDKMAEAYSEIGMKGERRRGKGHDGLYQGLS | |
| | TATKDTYDALHMQALPPR | |
| 17 | DIQMTQTTSSLSASLGDRVTISCRASQDISKYLNWYQQKPDGTVKLLIYHTSRLHSGV | 114 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFACDWIT | |
| | AVLPTVIICVMVFCLILWWRRKRKEKQSETSPKEFLTIYEDVKDLKTRRNHEQEQTFP | |
| | GGGSTIYSMIQSQSSAPTSTGGGSGGSRVKFSRSADAPAYQQGQNQLYNELNLGRREE | |
| | YDVLDKRRGRDPEMGGKPQRRKNPQEGLYNELQKDKMAEAYSEIGMKGERRRGKGHDG | |
| | LYQGLSTATKDTYDALHMQALPPR | |
| 18 | DIQMTQTTSSLSASLGDRVTISCRASQDISKYLNWYQQKPDGTVKLLIYHTSRLHSGV | 115 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFACDGLV | |
| | VAIVLIVLCLIGLIVAILRWPPSAACSGKESVVAIRTNSQSDFHLQTYGDEDLNELDP | |
| | HYEMRVKFSRSADAPAYQQGQNQLYNELNLGRREEYDVLDKRRGRDPEMGGKPQRRKN | |
| | PQEGLYNELQKDKMAEAYSEIGMKGERRRGKGHDGLYQGLSTATKDTYDALHMQALPP | |
| | R | |
| 19 | DIQMTQTTSSLSASLGDRVTISCRASQDISKYLNWYQQKPDGTVKLLIYHTSRLHSGV | 116 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSALSNSIMYFSHFVPVFLPAKPTTTPAPRPPTPAPTIASQPLSLRPEAC | |
| | RPAAGGAVHTRGLDFACDIYIWAPLAGTCGVLLLSLVITRVKFSRSADAPAYQQGQNQ | |
| | LYNELNLGRREEYDVLDKRRGRDPEMGGKPQRRKNPQEGLYNELQKDKMAEAYSEIGM | |
| | KGERRRGKGHDGLYQGLSTATKDTYDALHMQALPPR | |
| 20 | DIQMTQTTSSLSASLGDRVTISCRASQDISKYLNWYQQKPDGTVKLLIYHTSRLHSGV | 117 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSALSNSIMYFSHFVPVFLPAKPTTTPAPRPPTPAPTIASQPLSLRPEAC | |
| | RPAAGGAVHTRGLDFACDIYIWAPLAGTCGVLLLSLVITKWKKKKRPRNSYKCGTNTM | |
| | EREESEQTKKREKIHIPERSDEAQRVFKSSKTSSCDKSDTCFRVKFSRSADAPAYQQG | |
| | QNQLYNELNLGRREEYDVLDKRRGRDPEMGGKPQRRKNPQEGLYNELQKDKMAEAYSE | |
| | IGMKGERRRGKGHDGLYQGLSTATKDTYDALHMQALPPR | |
| 21 | DIQMTQTTSSLSASLGDRVTISCRASQDISKYLNWYQQKPDGTVKLLIYHTSRLHSGV | 118 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSALSNSIMYFSHFVPVFLPAKPTTTPAPRPPTPAPTIASQPLSLRPEAC | |
| | RPAAGGAVHTRGLDFACDVLLCLLLVPLLLSLFVLGLFLKWKKKKRPRNSYKCGTNTM | |
| | EREESEQTKKREKIHIPERSDEAQRVFKSSKTSSCDKSDTCFRVKFSRSADAPAYQQG | |
| | QNQLYNELNLGRREEYDVLDKRRGRDPEMGGKPQRRKNPQEGLYNELQKDKMAEAYSE | |
| | IGMKGERRRGKGHDGLYQGLSTATKDTYDALHMQALPPR | |
| 22 | DIQMTQTTSSLSASLGDRVTISCRASQDISKYLNWYQQKPDGTVKLLIYHTSRLHSGV | 119 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFACDIYI | |
| | WAPLAGTCGVLLLSLVITTYCFAPRCRERRRNERLRRESVRPVRVKFSRSADAPAYQQ | |
| | GQNQLYNELNLGRREEYDVLDKRRGRDPEMGGKPQRRKNPQEGLYNELQKDKMAEAYS | |
| | EIGMKGERRRGKGHDGLYQGLSTATKDTYDALHMQALPPR | |
| 23 | DIQMTQTTSSLSASLGDRVTISCRASQDISKYLNWYQQKPDGTVKLLIYHTSRLHSGV | 120 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFACDVLL | |
| | CLLLVPLLLSLFVLGLFLTTYCFAPRCRERRRNERLRRESVRPVRVKFSRSADAPAYQ | |
| | QGQNQLYNELNLGRREEYDVLDKRRGRDPEMGGKPQRRKNPQEGLYNELQKDKMAEAY | |
| | SEIGMKGERRRGKGHDGLYQGLSTATKDTYDALHMQALPPR | |
| 24 | DIQMTQTTSSLSASLGDRVTISCRASQDISKYLNWYQQKPDGTVKLLIYHTSRLHSGV | 121 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFACDIYI | |
| | WAPLAGTCGVLLLSLVITTYCFAPRCRERRRNERLRRESVRPVRVKFSRSADAPAYQQ | |
| | GQNQLYNELNLGRREEYDVLDKRRGRDPEMGGKPQRRKNPQEGLYNELQKDKMAEAYS | |
| | EIGMKGERRRGKGHDGLYQGLSTATKDTYDALHMQALPPR | |
| 25 | MALPVTALLLPLALLLHAARPDIQMTQTTSSLSASLGDRVTISCRASQDISKYLNWYQ | 100 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDVLLCLLLVPLLLSLFVLGLFLKWKKKKRPRNSYKCGTNTM | |
| | EREESEQTKKREKIHIPERSDEAQRVFKSSKTSSCDKSDTCFRVKFSRSADAPAYQQG | |
| | QNQLYNELNLGRREEYDVLDKRRGRDPEMGGKPQRRKNPQEGLYNELQKDKMAEAYSE | |
| | IGMKGERRRGKGHDGLYQGLSTATKDTYDALHMQALPPR | |
| 26 | MALPVTALLLPLALLLHAARPDIQMTQTTSSLSASLGDRVTISCRASQDISKYLNWYQ | 101 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDWITAVLPTVIICVMVFCLILWWRRKRKEKQSETSPKEFLT | |
| | IYEDVKDLKTRRNHEQEQTFPGGGSTIYSMIQSQSSAPTSTGGGSGGSRVKFSRSADA | |
| | PAYQQGQNQLYNELNLGRREEYDVLDKRRGRDPEMGGKPQRRKNPQEGLYNELQKDKM | |
| | AEAYSEIGMKGERRRGKGHDGLYQGLSTATKDTYDALHMQALPPR | |
| 27 | MALPVTALLLPLALLLHAARPDIQMTQTTSSLSASLGDRVTISCRASQDISKYLN | 131 |
| | WYQQKPDGTVKLLIYHTSRLHSGVPSRFSGSGSGTDYSLTISNLEQEDIATYFCQ | |
| | QGNTLPYTFGGGTKLEITGGGGSGGGGSGGGGSEVKLQESGPGLVAPSQSLSVTC | |
| | TVSGVSLPDYGVSWIRQPPRKGLEWLGVIWGSETTYYNSALKSRLTIIKDNSKSQ | |
| | VFLKMNSLQTDDTAIYYCAKHYYYGGSYAMDYWGQGTSVTVSSTTTPAPRPPTPA | |
| | PTIASQPLSLRPEACRPAAGGAVHTRGLDFACDGLVVAIVLIVLCLIGLIVAILR | |
| | WPPSAACSGKESVVAIRTNSQSDFHLQTYGDEDLNELDPHYEMRVKFSRSADAPA | |
| | YQQGQNQLYNELNLGRREEYDVLDKRRGRDPEMGGKPQRRKNPQEGLYNELQKDK | |
| | MAEAYSEIGMKGERRRGKGHDGLYQGLSTATKDTYDALHMQALPPR | |
| 28 | MALPVTALLLPLALLLHAARPDIQMTQTTSSLSASLGDRVTISCRASQDISKYLNWYQ | 104 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSALSNSIMYFSHFVPVFLPAKPTTTPAP | |
| | RPPTPAPTIASQPLSLRPEACRPAAGGAVHTRGLDFACDIYIWAPLAGTCGVLLLSLV | |
| | ITKWKKKKRPRNSYKCGTNTMEREESEQTKKREKIHIPERSDEAQRVFKSSKTSSCDK | |
| | SDTCFRVKFSRSADAPAYQQGQNQLYNELNLGRREEYDVLDKRRGRDPEMGGKPQRRK | |
| | NPQEGLYNELQKDKMAEAYSEIGMKGERRRGKGHDGLYQGLSTATKDTYDALHMQALP | |
| | PR | |
| 29 | MALPVTALLLPLALLLHAARPDIQMTQTTSSLSASLGDRVTISCRASQDISKYLNWYQ | 125 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDIYIWAPLAGTCGVLLLSLVITRVKFSRSADAPAYQQGQNQ | |
| | LYNELNLGRREEYDVLDKRRGRDPEMGGKPQRRKNPQEGLYNELQKDKMAEAYSEIGM | |
| | KGERRRGKGHDGLYQGLSTATKDTYDALHMQALPPR | |
| 30 | MALPVTALLLPLALLLHAARPDIQMTQTTSSLSASLGDRVTISCRASQDISKYLNWYQ | 126 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDALVVIPIIFGILFAILLVLVFIRVKFSRSADAPAYQQGQN | |
| | QLYNELNLGRREEYDVLDKRRGRDPEMGGKPQRRKNPQEGLYNELQKDKMAEAYSEIG | |
| | MKGERRRGKGHDGLYQGLSTATKDTYDALHMQALPPR | |
| 31 | MALPVTALLLPLALLLHAARPDIQMTQTTSSLSASLGDRVTISCRASQDISKYLNWYQ | 127 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDVVVACMSIMALLLLLLLLLLYRVKFSRSADAPAYQQGQNQ | |
| | LYNELNLGRREEYDVLDKRRGRDPEMGGKPQRRKNPQEGLYNELQKDKMAEAYSEIGM | |
| | KGERRRGKGHDGLYQGLSTATKDTYDALHMQALPPR | |
| 32 | MALPVTALLLPLALLLHAARPDIQMTQTTSSLSASLGDRVTISCRASQDISKYLNWYQ | 98 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDIYIWAPLAGTCGVLLLSLVITKWKKKKRPRNSYKCGTNTM | |
| | EREESEQTKKREKIHIPERSDEAQRVFKSSKTSSCDKSDTCFRVKFSRSADAPAYQQG | |
| | QNQLYNELNLGRREEYDVLDKRRGRDPEMGGKPQRRKNPQEGLYNELQKDKMAEAYSE | |
| | IGMKGERRRGKGHDGLYQGLSTATKDTYDALHMQALPPR | |
| 33 | MALPVTALLLPLALLLHAARPDIQMTQTTSSLSASLGDRVTISCRASQDISKYLNWYQ | 100 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDVLLCLLLVPLLLSLFVLGLFLKWKKKKRPRNSYKCGTNTM | |
| | EREESEQTKKREKIHIPERSDEAQRVFKSSKTSSCDKSDTCFRVKFSRSADAPAYQQG | |
| | QNQLYNELNLGRREEYDVLDKRRGRDPEMGGKPQRRKNPQEGLYNELQKDKMAEAYSE | |
| | IGMKGERRRGKGHDGLYQGLSTATKDTYDALHMQALPPR | |
| 34 | MALPVTALLLPLALLLHAARPDIQMTQTTSSLSASLGDRVTISCRASQDISKYLNWYQ | 101 |
| | QKPDGTVKLLIYHTSRLHSGVPSRFSGSGSGTDYSLTISNLEQEDIATYFCQQGNTLP | |
| | YTFGGGTKLEITGGGGSGGGGSGGGGSEVKLQESGPGLVAPSQSLSVTCTVSGVSLPD | |
| | YGVSWIRQPPRKGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIASQPLSLRPEAC | |
| | RPAAGGAVHTRGLDFACDWITAVLPTVIICVMVFCLILWWRRKRKEKQSETSPKEFLT | |
| | IYEDVKDLKTRRNHEQEQTFPGGGSTIYSMIQSQSSAPTSTGGGSGGSRVKFSRSADA | |
| | PAYQQGQNQLYNELNLGRREEYDVLDKRRGRDPEMGGKPQRRKNPQEGLYNELQKDKM | |
| | AEAYSEIGMKGERRRGKGHDGLYQGLSTATKDTYDALHMQALPPR | |
| 35 | DIQMTQTTSSLSASLGDRVTISCRASQDISKYLNWYQQKPDGTVKLLIYHTSRLHSGV | 128 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFACDIYI | |
| | WAPLAGTCGVLLLSLVITRVKFSRSADAPAYQQGQNQLYNELNLGRREEYDVLDKRRG | |
| | RDPEMGGKPQRRKNPQEGLYNELQKDKMAEAYSEIGMKGERRRGKGHDGLYQGLSTAT | |
| | KDTYDALHMQALPPR | |
| 36 | DIQMTQTTSSLSASLGDRVTISCRASQDISKYLNWYQQKPDGTVKLLIYHTSRLHSGV | 129 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFACDALV | |
| | VIPIIFGILFAILLVLVFIRVKFSRSADAPAYQQGQNQLYNELNLGRREEYDVLDKRR | |
| | GRDPEMGGKPQRRKNPQEGLYNELQKDKMAEAYSEIGMKGERRRGKGHDGLYQGLSTA | |
| | TKDTYDALHMQALPPR | |
| 37 | DIQMTQTTSSLSASLGDRVTISCRASQDISKYLNWYQQKPDGTVKLLIYHTSRLHSGV | 130 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFACDVVV | |
| | ACMSIMALLLLLLLLLLYRVKFSRSADAPAYQQGQNQLYNELNLGRREEYDVLDKRRG | |
| | RDPEMGGKPQRRKNPQEGLYNELQKDKMAEAYSEIGMKGERRRGKGHDGLYQGLSTAT | |
| | KDTYDALHMQALPPR | |
| 38 | DIQMTQTTSSLSASLGDRVTISCRASQDISKYLNWYQQKPDGTVKLLIYHTSRLHSGV | 132 |
| | PSRFSGSGSGTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRKGLEWLGVIW | |
| | GSETTYYNSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFACDGLV | |
| | VAIVLIVLCLIGLIVAILRWPPSAACSGKESVVAIRTNSQSDFHLQTYGDEDLNELDP | |
| | HYEMRVKFSRSADAPAYQQGQNQLYNELNLGRREEYDVLDKRRGRDPEMGGKPQRRKN | |
| | PQEGLYNELQKDKMAEAYSEIGMKGERRRGKGHDGLYQGLSTATKDTYDALHMQALPP | |
| | R | |

5.5. Modified cells

As provided herein, in some aspects the present disclosure involves modified cells comprising a CAR (such as a CAR described in Section 5.1), a polynucleotide encoding a CAR (such as a polynucleotide described in Section 5.2), a vector comprising a polynucleotide encoding a CAR (such as a vector described in Section 5.3), or a CAR polypeptide (such as a polypeptide described in Section 5.4). In specific embodiments, the modified cells provided herein are generated according to the methods provided herein, such as the methods of generating a modified cell described in Section 5.8.

In certain aspects, the present disclosure relates to modified mammalian cells. In some embodiments, provided herein are modified human cells. In some embodiments, the modified human cells are autologous or allogeneic to a subject receiving administration of the modified cells. In some embodiments, the modified cells are allogeneic, relative to the subject. In other embodiments, the modified cells are autologous, relative to the subject. In some embodiments, provided herein are modified non-human mammalian cells.

The present disclosure relates, in part, to modified cells that are monocytes or macrophages. In some embodiments, the modified cell is a monocyte. In specific embodiments, the monocyte is derived from an induced pluripotent stem cell (iPSC). For example, in some embodiments, the modified cell is a modified iPSC generated according to the methods provided herein, such as the methods described in Section 5.8, and the modified iPSC is differentiated into a monocyte. In other embodiments, the monocyte is derived from an iPSC and the iPSC derived monocyte is modified according to the methods provided herein, such as the methods described in Section 5.8. Techniques known to one of skill in the art may be used to differentiate an iPSC into a monocyte. See, for example, Lyadova, et al. (Front Cell Dev Biol., 2021; 9:640703).

In some embodiments, the monocyte is an isolated monocyte. Techniques known to one of skill in the art may be used to isolate monocytes. For example, monocytes can be isolated from the peripheral blood lymphocytes using techniques known to one of skill in the art or described herein. An exemplary isolation method can include plastic adhesion or magnetic bead-based immuno-isolation kits (negative and CD14pos selection). In some embodiments, monocytes are isolated based on their expression of CD14 and CD16. In some embodiments, a subset of monocytes are isolated based on CD14^{high} CD16^{neg} expression. In some embodiments, a subset of monocytes are isolated based on CD14^{high} CD16^{pos} expression. In some embodiments, a subset of monocytes are isolated based on CD14^{low} CD16^{high} expression. Alternatively, or in addition, depletion of non-monocytes can be used, such as removal of thrombocytes, to reduce platelet, lymphocyte, and granulocyte contamination. In some embodiments, the isolated monocyte is 50%, 60%, 70%, 80%, 90%, 95%, 99%, or 100% pure of other cells and/or substances.

In some embodiments, the modified cell is a macrophage. In specific embodiments, the modified macrophage is derived from an induced pluripotent stem cell (iPSC). For example, in some embodiments, the modified cell is a modified iPSC generated according to the methods provided herein, such as the methods described in Section 5.8, and the modified iPSC is differentiated into a macrophage. In other embodiments, the macrophage is derived from an iPSC and the iPSC derived macrophage is modified according to the methods provided herein, such as the methods described in Section 5.8. In some embodiments, the macrophage is derived from a modified monocyte that is modified according to the methods provided herein, such as the methods described in Section 5.8. Techniques known to one of skill in the art or described herein (e.g., the Examples) may be used to differentiate an iPSC into a macrophage, and/or differentiate a monocyte into a macrophage. See, for example, Lyadova, et al. (Front Cell Dev Biol., 2021; 9:640703).

In some embodiments, the macrophage is an isolated macrophage. Techniques known to one of skill in the art may be used to isolate macrophages. In some embodiments, a subset of macrophages are isolated based on cell size and morphology. In some embodiments, the isolated macrophage is 50%, 60%, 70%, 80%, 90%, 95%, 99%, or 100% pure of other cells and/or substances.

In preferred embodiments, the macrophage is a pro-inflammatory macrophage (i.e., an M1 macrophage). In some embodiments, the macrophage secretes TNFα, IL-17A, and/or type 1 cytokines (e.g., IL-6, or IL-12). In some embodiments, the macrophage expresses a marker in Table 8. In some embodiments, the macrophage do not express a marker in Table 3. In some embodiments, expression of the CAR promotes M1 polarization of the macrophage.

TABLE 8
Human M1 macrophage markers

| Species | Macrophage type | Marker | Marker type |
|---|---|---|---|
| Human | M1 | IFN-γ | Secreted |
| Human | M1 | IL-1α | Secreted |
| Human | M1 | IL-1β | Secreted |
| Human | M1 | IL-6 | Secreted |
| Human | M1 | IL-12 | Secreted |
| Human | M1 | IL-23 | Secreted |
| Human | M1 | TNF-α | Secreted |
| Human | M1 | CD16 | Surface |
| Human | M1 | CD16/CD32 | Surface |
| Human | M1 | CD32 | Surface |
| Human | M1 | CD64 | Surface |
| Human | M1 | CD68 | Surface |
| Human | M1 | CD80 | Surface |
| Human | M1 | CD86 | Surface |
| Human | M1 | CD369 | Surface |
| | | (DECTIN-1) | |
| Human | M1 | Mer (MerTK) | Surface |
| Human | M1 | MHC II | Surface |
| Human | M1 | IRF5 | Intracellular/ |
| | | | transcription |
| | | | factor |
| Human | M1 | STATI | Intracellular/ |
| | | | transcription |
| | | | factor |

In some embodiments, the macrophage is not an anti-inflammatory macrophage (i.e., an M2 macrophage). In specific embodiments, the macrophage does not secrete IL-4, IL-13, and/or type 2 cytokines. In some embodiments, the macrophage does not express a marker in Table 9. In specific embodiments, expression of the CAR decreases expression of one of more M2 marker.

TABLE 9
Human Macrophage M2 markers.

| Species | Macrophage type | Marker | Marker type |
|---|---|---|---|
| Human | M2 | IDO | Secreted |
| Human | M2 | IL-10 | Secreted |
| Human | M2 | TGF-b | Secreted |
| Human | M2 | CD115 | Surface |
| Human | M2 | CD204 | Surface |
| Human | M2 | CD163 | Surface |
| Human | M2 | CD206 (MMR) | Surface |
| Human | M2 | CD209 (DC-SIGN) | Surface |
| Human | M2 | FceR1 | Surface |
| Human | M2 | VSIG4 | Surface |
| Human | M2 | IRF4 | Intracellular/ |
| | | | transcription |
| | | | factor |
| Human | M2 | STAT6 | Intracellular/ |
| | | | transcription |
| | | | factor |

The present disclosure also relates, in part, to modified cells that are iPSCs. For example, in some embodiments, the modified cell is a modified iPSC generated according to the methods provided herein, such as the methods described in Section 5.8. The use of modified iPSCs as the starting material can not only aide in overcoming barriers to macrophage expansion, but also facilitate generation of a homogeneous monocytes/macrophage cell population. Accordingly, in some embodiments, about 80% or more of the modified cells express a CAR, such as a CAR described in Section 5.1. In some embodiments, about 85% or more of the modified cells express a CAR, such as a CAR described in Section 5.1. In some embodiments, about 90% or more of the modified cells express a CAR, such as a CAR described in Section 5.1. In some embodiments, about 95% or more of the modified cells express a CAR, such as a CAR described in Section 5.1. In some embodiments, about 99% or more of the modified cells express a CAR, such as a CAR described in Section 5.1. In some embodiments, 100% of the modified cells express a CAR, such as a CAR described in Section 5.1.

In certain aspects, the present disclosure relates to modified mammalian cells comprising a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the modified mammalian cells are modified human cells comprising a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. Various techniques known to one of skill in the art or described herein may be used to disrupt a gene. Exemplary techniques include genome editing tools, such as TALEN (Transcription Activator-Like Effector Nucleases), Zinc-finger nucleases (ZFN), and CRISPR, as well as variations thereof (e.g., base editing, prime editing, etc.); RNA interference (RNAi) (e.g., shRNA, or siRNA), or Cre/LoxP-based conditional knockout. In certain embodiments, genetic disruption deletes expression of the corresponding protein encoded by the gene. In some embodiments, genetic disruption prevents or reduces expression of the full-length protein encoded by the gene. In certain embodiments, a polynucleotide encoding a CAR (such as a polynucleotide described in Section 5.2) is integrated into (a) a SIRPA gene; (b) a CISH gene; and/or (c) a SIGLEC10 gene, and the modified mammalian cell comprises genetic disruption of the gene(s) in which the polynucleotide encoding the CAR is integrated.

In some embodiments, the gene editing system comprises a CRISPR system. In some embodiments, the CRISPR system comprises a Class 2 CRISPR system. Class 2 systems currently represent a single protein that is categorized into three distinct types (types II, V and VI). Any class 2 CRISPR system suitable for gene editing, for example a type II, a type V or a type VI system, is envisaged as within the scope of the instant disclosure. Exemplary Class 2 type II CRISPR systems include Cas9, Csn2 and Cas4. Exemplary Class 2, type V CRISPR systems include, Cas12, Cas12a (e.g., Cpf1, MAD7), Cas12b (C2c1), Cas12c (C2c3), Cas12d (CasY), Cas12e (CasX), Cas12f, Cas12g, Cas12h, Cas12i and Cas12k (C2c5). Exemplary Class 2 Type VI systems include Cas13, Cas13a (C2c2) Cas13b, Cas13c and Cas13d.

The endonuclease protein (e.g., nucleic acid-directed nuclease) may be derived from any bacterial or archaeal Cas protein. Any suitable CRISPR system is contemplated as within the scope of the instant disclosure. In some embodiments, the endonuclease protein comprises one or more of Cas1, Cas1B, Cas2, Cas3, Cas4, Cas5, Cas6, Cas7, Cas8, Cas9, Cas10, Cas12, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the endonuclease protein is a Cas9 protein, a Cpf1 protein, a C2cl protein, a C2c2 protein, a C2c3 protein, Cas3, Cas3-HD, Cas5, Cas7, Cas8, Cas10, Cas12, modified versions thereof, or combinations or complexes of these.

In some embodiments, the Class 2 CRISPR system comprises a type V system. In some embodiments, the Type V system comprises a Cas12a CRISPR/Cas protein. Exemplary Cas12a proteins include MAD7, isolated from Eubacterium rectale. MAD7 uses T-rich protospacer adjacent motifs (PAMs) such as YTTN. Exemplary MAD7 proteins are described in US20190360001 and WO 2021119563, the contents of which are incorporated by reference herein in their entireties.

In some embodiments, the modified cells provided herein comprise genetic disruption of SIRPA (e.g., Homo sapiens NCBI Gene ID: 140885, Mus musculus NCBI Gene ID: 19261). SIRPA encodes a SIRPα polypeptide, an immunoglobulin-like cell surface receptor for CD47. In certain embodiments, genetic disruption of SIRPA prevents or reduces expression of a SIRPα polypeptide capable of interaction with CD47. In certain embodiments, genetic disruption of SIRPA prevents a SIRPα polypeptide from being capable of interaction with CD47. In some embodiments, the SIRPA gene is a human SIRPA gene. In some embodiments, the SIRPA gene is a non-human SIRPA gene (e.g., a mouse SIRPA gene).

In some embodiments, genetic disruption of SIRPA is performed by a genome editing tool, such as TALEN, ZFN, or CRISPR (e.g., by deletion of one or more exons, introduction of a stop codon, introduction of a null mutation or inactivation of the promoter). In some embodiments, genetic disruption of SIRPA is performed using gRNA. In certain embodiments, genetic disruption of SIRPA is performed using gRNA and a Cas12a protein. In certain embodiments, genetic disruption is performed using gRNA and a MAD7 protein. In some embodiments, the gRNA comprises a targeting sequence complementary to a SIRPA target sequence. In some embodiments, the SIRPA target sequence comprises coding sequence, for example SIRPA mRNA sequence. In some embodiments, the gRNA targets exon 1 of SIRPA. In some embodiments, the gRNA targets exon 2 of SIRPA. For example, exon 2 of SIRPA can be targeted with a guide RNA (gRNA), such as a guide RNA sequence 5′-CGACCUCUCUGAUCCCUGUG-3′ that targets the genetic sequence 5′-TGACCTCCCTGATCCCTGTG-3′. In some embodiments, the gRNA targets exon 3 of SIRPA. In some embodiments, the gRNA targets exon 4 of SIRPA. In some embodiments, the gRNA targets exon 5 of SIRPA. In some embodiments, the gRNA targets exon 6 of SIRPA. In some embodiments, the gRNA targets exon 7 of SIRPA. In some embodiments, the gRNA targets exon 8 of SIRPA. In some embodiments, the gRNA targets exon 9 of SIRPA. In some embodiments, the gRNA targets exon 10 of SIRPA. In some embodiments, the gRNA targets exon 11 of SIRPA. In some embodiments, the gRNA targets exon 12 of SIRPA. In some embodiments, the SIRPA target sequence comprises non-coding sequence. Exemplary non-coding sequence includes SIRPA intronic, promoter, 5′ untranslated region (UTR), 3′ UTR, or enhancer sequence. In some embodiments, genetic disruption of SIRPA is performed by RNAi. In some embodiments, the genetic disruption of SIRPA is performed by conditional knockout. In some embodiments, the genetic disruption of SIRPA is performed in human cells.

In some embodiments, the modified cells comprise an agent capable of preventing or reducing interaction between CD47 and SIRPα (see, e.g., WO 2019/241403, incorporated by reference in its entirety). In some embodiments, the agent comprises an anti-CD47 antibody. Non-limiting examples of suitable anti-CD47 antibodies include clones B6H12, 5F9, 8B6, and C3 (see, e.g., WO 2011/143624, incorporated by reference in its entirety). In some embodiments, the agent comprises a soluble CD47 polypeptide. In some embodiments, the agent comprises an anti-SIRPα antibody.

In some embodiments, the modified cells provided herein comprise genetic disruption of CISH (e.g., Homo sapiens NCBI Gene ID: 1154, Mus musculus NCBI Gene ID: 12700). CISH is an inhibitory immune checkpoint gene. In certain embodiments, genetic disruption of CISH prevents or reduces expression of a CISH polypeptide. In some embodiments, the CISH gene is a human CISH gene. In some embodiments, the CISH gene is a non-human CISH gene (e.g., a mouse CISH gene).

In some embodiments, genetic disruption of CISH is performed by a genome editing tool, such as TALEN, ZFN, or CRISPR (e.g., by deletion of one or more exons, introduction of a stop codon, introduction of a null mutation or inactivation of the promoter). In some embodiments, genetic disruption of CISH is performed using gRNA. In certain embodiments, genetic disruption of CISH is performed using gRNA and a Cas12a protein. In certain embodiments, genetic disruption is performed using gRNA and a MAD7 protein. In some embodiments, the gRNA comprises a targeting sequence complementary to a CISH target sequence. In some embodiments, the CISH target sequence comprises coding sequence, for example CISH mRNA sequence. In some embodiments, the gRNA targets exon 1 of CISH. In some embodiments, the gRNA targets exon 2 of CISH. In some embodiments, the gRNA targets exon 3 of CISH. In some embodiments, the gRNA targets exon 4 of CISH. In some embodiments, the CISH target sequence comprises non-coding sequence. Exemplary non-coding sequence includes CISH intronic, promoter, 5′ untranslated region (UTR), 3′ UTR, or enhancer sequence. In some embodiments the gRNA is compatible with a gRNA/Cas9 system. In some embodiments the gRNA is compatible with a gRNA/Cas12a (e.g., MAD7) system. In some embodiments, genetic disruption of CISH is performed by RNAi. In some embodiments, genetic disruption of CISH is performed by conditional knockout. Non-limiting examples of CISH inhibitors, including CISH guide RNAs (gRNAs), include those provided in WO 2017/100861, WO 2017/023803, WO 2018/075664, WO 2019/213610, and WO 2019/217956, each of which are incorporated by reference in its entirety. In some embodiments, the genetic disruption of CISH is performed in human cells.

In some embodiments, the modified cells provided herein comprise genetic disruption of SIGLEC10 (e.g., Homo sapiens NCBI Gene ID: 89790, Mus musculus NCBI Gene ID: 243958). SIGLEC10 is a ligand for CD52, vascular adhesion protein 1 (VAP-1), and CD24. The CD24-SIGLEC10 interaction can serve as an anti-phagocytic signal (see, e.g., Barkal A A, et al., Nature. 2019 August; 572 (7769): 392-396). Thus, in some embodiments genetic disruption of SIGLEC10 prevents or reduces expression of a SIGLEC10 polypeptide capable of interaction with CD24 on a target cell. In some embodiments genetic disruption of SIGLEC10 prevents a SIGLEC10 polypeptide from being capable of interaction with CD24 on a target cell. In some embodiments, the SIGLEC10 gene is a human SIGLEC10 gene. In some embodiments, the SIGLEC10 gene is a non-human SIGLEC10 gene (e.g., a mouse SIGLEC10 gene).

In some embodiments, genetic disruption of SIGLEC10 is performed by a genome editing tool, such as TALEN, ZFN, or CRISPR (e.g., by deletion of one or more exons, introduction of a stop codon, introduction of a null mutation or inactivation of the promoter). In some embodiments, genetic disruption of SIGLEC10 is performed using gRNA. In certain embodiments, genetic disruption of SIGLEC10 is performed using gRNA and a Cas12a protein. In certain embodiments, genetic disruption is performed using gRNA and a MAD7 protein. In some embodiments, the gRNA comprises a targeting sequence complementary to a SIGLEC10 target sequence. In some embodiments, the SIGLEC10 target sequence comprises coding sequence, for example SIGLEC10 mRNA sequence. In some embodiments, the gRNA targets exon 1 of SIGLEC10. In some embodiments, the gRNA targets exon 2 of SIGLEC10. In some embodiments, the gRNA targets exon 3 of SIGLEC10. In some embodiments, the gRNA targets exon 4 of SIGLEC10. In some embodiments, the gRNA targets exon 5 of SIGLEC10. In some embodiments, the gRNA targets exon 6 of SIGLEC10. In some embodiments, the gRNA targets exon 7 of SIGLEC10. In some embodiments, the gRNA targets exon 8 of SIGLEC10. In some embodiments, the gRNA targets exon 9 of SIGLEC10. In some embodiments, the gRNA targets exon 10 of SIGLEC10. In some embodiments, the gRNA targets exon 11 of SIGLEC10. In some embodiments, the SIGLEC10 target sequence comprises non-coding sequence. Exemplary non-coding sequence includes SIGLEC10 intronic, promoter, 5′ untranslated region (UTR), 3′ UTR, or enhancer sequence. In some embodiments, genetic disruption of SIGLEC10 is performed by RNAi. In some embodiments, genetic disruption of SIGLEC10 is performed by conditional knockout. In some embodiments, the genetic disruption of SIGLEC10 is performed in human cells.

In some embodiments, the modified cell comprises an agent capable of preventing or reducing interaction between CD24 and SIGLEC10 (see, e.g., WO 2019/241403, incorporated by reference in its entirety). In some embodiments, the agent comprises an anti-CD24 antibody. In some embodiments, the agent comprises an anti-SIGLEC10 antibody. In some embodiments, the agent comprises a soluble SIGLEC10 polypeptide.

In specific embodiments, the modified cells provided herein comprise genetic disruption of SIRPA and CISH. In certain embodiments, the modified cells provided herein comprise genetic disruption of SIRPA and SIGLEC10. In further embodiments, the modified cells provided herein comprise genetic disruption of SIGLEC10 and CISH. In still further embodiments, the modified cells provided herein comprise genetic disruption of SIGLEC10, CISH, and SIRPA. In some embodiments, genetic disruption prevents or reduces expression of the full-length protein encoded by the gene. In certain embodiments, the modified cells provided herein comprise a polynucleotide encoding a CAR (such as a polynucleotide described in Section 5.2) that is integrated into (a) a SIRPA gene; (b) a CISH gene; and/or (c) a SIGLEC10 gene, and the modified cell comprises genetic disruption of the gene(s) in which the polynucleotide encoding the CAR is integrated.

### 5.6. Compositions

In one aspect, provided herein is a composition comprising a modified cell of the present disclosure (such as a modified cell described in Section 5.5). In some embodiments, provided herein is a composition comprising a modified monocyte of the present disclosure (such as a modified monocyte described in Section 5.5). In some embodiments, provided herein is a composition comprising a modified macrophage of the present disclosure (such as a modified macrophage described in Section 5.5). In some embodiments, provided herein is a composition comprising a modified iPSC of the present disclosure (such as a modified iPSC described in Section 5.5). In some embodiments, the modified cell provided herein (such as a such as a modified cell described in Section 5.5) comprises a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the modified cell provided herein comprises genetic disruption of SIRPA. In some embodiments, the modified cell provided herein comprises genetic disruption of CISH. In some embodiments, the modified cell provided herein comprises genetic disruption of SIGLEC10. In specific embodiments, the modified cell provided herein comprises genetic disruption of SIRPA and CISH. In certain embodiments, the modified cell provided herein comprises genetic disruption of SIRPA and SIGLEC10. In further embodiments, the modified cell provided herein comprises genetic disruption of SIGLEC10 and CISH. In still further embodiments, the modified cell provided herein comprises genetic disruption of SIGLEC10, CISH, and SIRPA. In certain embodiments, the modified cell provided herein comprises a polynucleotide encoding a CAR (such as a polynucleotide described in Section 5.2) that is integrated into (a) a SIRPA gene; (b) a CISH gene; and/or (c) a SIGLEC10 gene, and the modified cell comprises genetic disruption of the gene(s) in which the polynucleotide encoding the CAR is integrated.

In another aspect, provided herein is a pharmaceutical composition comprising a modified cell of the present disclosure (such as a modified cell described in Section 5.5) and a pharmaceutically acceptable carrier. In some embodiments, provided herein is a pharmaceutical composition comprising a modified monocyte of the present disclosure (such as a modified monocyte described in Section 5.5) and a pharmaceutically acceptable carrier. In some embodiments, provided herein is a pharmaceutical composition comprising a modified macrophage of the present disclosure (such as a modified macrophage described in Section 5.5) and a pharmaceutically acceptable carrier. In some embodiments, provided herein is a pharmaceutical composition comprising a modified iPSC of the present disclosure (such as a modified iPSC described in Section 5.5) and a pharmaceutically acceptable carrier. In some embodiments, the modified cell provided herein comprises a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the modified cell provided herein comprises genetic disruption of SIRPA. In some embodiments, the modified cell provided herein comprises genetic disruption of CISH. In some embodiments, the modified cell provided herein comprises genetic disruption of SIGLEC10. In specific embodiments, the modified cell provided herein comprises genetic disruption of SIRPA and CISH. In certain embodiments, the modified cell provided herein comprises genetic disruption of SIRPA and SIGLEC10. In further embodiments, the modified cell provided herein comprises genetic disruption of SIGLEC10 and CISH. In still further embodiments, the modified cell provided herein comprises genetic disruption of SIGLEC10, CISH, and SIRPA. In certain embodiments, the modified cell provided herein comprises a polynucleotide encoding a CAR (such as a polynucleotide described in Section 5.2) that is integrated into (a) a SIRPA gene; (b) a CISH gene; and/or (c) a SIGLEC10 gene, and the modified cell comprises genetic disruption of the gene(s) in which the polynucleotide encoding the CAR is integrated.

In yet another aspect, provided herein is a pharmaceutical composition comprising an effective amount of a modified cell of the present disclosure (such as a modified cell described in Section 5.5) and a pharmaceutically acceptable carrier. In some embodiments, provided herein is a pharmaceutical composition comprising an effective amount of a modified monocyte of the present disclosure (such as a modified monocyte described in Section 5.5) and a pharmaceutically acceptable carrier. In some embodiments, provided herein is a pharmaceutical composition comprising an effective amount of a modified macrophage of the present disclosure (such as a modified macrophage described in Section 5.5) and a pharmaceutically acceptable carrier. In some embodiments, provided herein is a pharmaceutical composition comprising an effective amount of a modified iPSC of the present disclosure (such as a modified iPSC described in Section 5.5) and a pharmaceutically acceptable carrier. In some embodiments, the modified cell provided herein comprises a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the modified cell provided herein comprises genetic disruption of SIRPA. In some embodiments, the modified cell provided herein comprises genetic disruption of CISH. In some embodiments, the modified cell provided herein comprises genetic disruption of SIGLEC10. In specific embodiments, the modified cell provided herein comprises genetic disruption of SIRPA and CISH. In certain embodiments, the modified cell provided herein comprises genetic disruption of SIRPA and SIGLEC10. In further embodiments, the modified cell provided herein comprises genetic disruption of SIGLEC10 and CISH. In still further embodiments, the modified cell provided herein comprises genetic disruption of SIGLEC10, CISH, and SIRPA. In certain embodiments, the modified cell provided herein comprises a polynucleotide encoding a CAR (such as a polynucleotide described in Section 5.2) that is integrated into (a) a SIRPA gene; (b) a CISH gene; and/or (c) a SIGLEC10 gene, and the modified cell comprises genetic disruption of the gene(s) in which the polynucleotide encoding the CAR is integrated.

As used herein, the term “pharmaceutically acceptable” when used in reference to a carrier, is intended to mean that the carrier, diluent or excipient is not toxic or otherwise undesirable, (i.e., the material may be administered to a subject without causing any undesirable biological effects), and it is compatible with the other ingredients of the formulation. The term “carrier” refers to a diluent, adjuvant, excipient, or vehicle with which the therapeutic is administered. Such pharmaceutical carriers can be sterile liquids, such as saline solutions. A saline solution can be a carrier when the pharmaceutical composition is administered intravenously. Saline solutions and aqueous dextrose and glycerol solutions can also be employed as liquid carriers, particularly for injectable solutions. The composition, if desired, can also contain minor amounts of wetting or emulsifying agents, or pH buffering agents.

In some embodiments, the pharmaceutical composition comprising a modified cell of the present disclosure (such as a modified cell described in Section 5.5) and a pharmaceutically acceptable carrier further comprises one or more agents. In some embodiments, the agent comprises an anti-CD24 antibody. In some embodiments, the agent comprises an anti-SIGLEC10 antibody. In some embodiments, the agent comprises an anti-CD47 antibody. Non-limiting examples of suitable anti-CD47 antibodies include clones B6H12, 5F9, 8B6, and C3 (see, e.g., WO 2011/143624, incorporated by reference in its entirety). In some embodiments, the agent comprises a soluble CD47 polypeptide. In some embodiments, the agent comprises an anti-SIRPαantibody. In some embodiments, the agent comprises an anti-CD20 antibody (e.g., rituximab). In some embodiments, the agent comprises an anti-HER2/neu antibody (e.g., trastuzumab). In certain embodiments, the modified cell of the present disclosure is precomplexed with one or more agents.

In some embodiments, the In some embodiments, provided herein is a pharmaceutical composition comprising an effective amount of a modified monocyte of the present disclosure (such as a modified monocyte described in Section 5.5) and a pharmaceutically acceptable carrier, further comprises one or more agents. In some embodiments, the agent comprises an anti-CD24 antibody. In some embodiments, the agent comprises an anti-SIGLEC10 antibody. In some embodiments, the agent comprises an anti-CD47 antibody. Non-limiting examples of suitable anti-CD47 antibodies include clones B6H12, 5F9, 8B6, and C3 (see, e.g., WO 2011/143624, incorporated by reference in its entirety). In some embodiments, the agent comprises a soluble CD47 polypeptide. In some embodiments, the agent comprises an anti-SIRPα antibody. In some embodiments, the agent comprises an anti-CD20 antibody (e.g., rituximab). In some embodiments, the agent comprises an anti-HER2/neu antibody (e.g., trastuzumab). In certain embodiments, the modified cell of the present disclosure is precomplexed with one or more agents.

### 5.7. Methods for Treatment

As provided herein, the modified cells of the present disclosure that comprise a CAR are suitable for use as a cell-based therapy or therapies, such as in a method of treating cancer. As provided herein, the modified cells of the present disclosure that comprise a CAR are suitable for use in a method of treating autoimmune disorders. As provided herein, the modified cells of the present disclosure that comprise a CAR are suitable for use in a method of treating fibrosis. For example, in some embodiments, an effective amount of the modified cells described in Section 5.5, can be combined with a pharmaceutically acceptable carrier to result in a pharmaceutical composition. In some embodiments, the cells provided herein comprise a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the cells provided herein comprise genetic disruption of SIRPA. In some embodiments, the cells provided herein comprise genetic disruption of CISH. In some embodiments, the cells provided herein comprise genetic disruption of SIGLEC10. In specific embodiments, the cells provided herein comprise genetic disruption of SIRPA and CISH. In certain embodiments, the cells provided herein comprise genetic disruption of SIRPA and SIGLEC10. In further embodiments, the cells provided herein comprise genetic disruption of SIGLEC10 and CISH. In still further embodiments, the cells provided herein comprise genetic disruption of SIGLEC10, CISH, and SIRPA. In certain embodiments, the modified cell provided herein comprises a polynucleotide encoding a CAR (such as a polynucleotide described in Section 5.2) that is integrated into (a) a SIRPA gene; (b) a CISH gene; and/or (c) a SIGLEC10 gene, and the modified cell comprises genetic disruption of the gene(s) in which the polynucleotide encoding the CAR is integrated.

In certain embodiments, treatment with modified cells of the present disclosure results in one, two, or more, or all of the following: (1) a reduction in severity, progression, spread, and/or frequency of one or more symptoms, (2) elimination of one or more symptoms and/or underlying cause, (3) prevention of the occurrence of one or more symptoms and/or their underlying cause, and (4) improvement or remediation of damage. In specific embodiments, treatment includes therapeutic treatment as well as prophylactic, or suppressive measures for the condition, disease or disorder.

In some embodiments, the modified cells of the present disclosure for use as cell-based therapy or therapies, such as in a method of treating cancer, are autologous or allogeneic to the subject receiving administration of the modified cells. In some embodiments, the modified cells of the present disclosure for use as a cell-based therapies, such as in a method of treating cancer, are autologous to the subject receiving administration of the modified cells. In some embodiments, the modified cells of the present disclosure for use as a cell-based therapies, such as in a method of treating cancer, are allogeneic to the subject receiving administration of the modified cells.

In some embodiments, the modified cells of the present disclosure for use as in a method of treating autoimmune disorders, are autologous or allogeneic to the subject receiving administration of the modified cells. In some embodiments, the modified cells of the present disclosure for use as in a method of treating autoimmune disorders, are autologous to the subject receiving administration of the modified cells. In some embodiments, the modified cells of the present disclosure for use as in a method of treating autoimmune disorders, are allogeneic to the subject receiving administration of the modified cells.

In some embodiments, the modified cells of the present disclosure for use as in a method of treating fibrosis, are autologous or allogeneic to the subject receiving administration of the modified cells. In some embodiments, the modified cells of the present disclosure for use as in a method of treating fibrosis, are autologous to the subject receiving administration of the modified cells. In some embodiments, the modified cells of the present disclosure for use as in a method of treating fibrosis, are allogeneic to the subject receiving administration of the modified cells.

In one aspect, provided herein is a method of treating a disease or disorder comprising administering a pharmaceutical composition (such as a pharmaceutical composition described in Section 5.6). In some embodiments, provided herein is a method for treating cancer in a subject in need thereof, comprising administering a pharmaceutical composition (such as a pharmaceutical composition described in Section 5.6). In some embodiments, provided herein is a method for treating autoimmune disorders in a subject in need thereof, comprising administering a pharmaceutical composition (such as a pharmaceutical composition described in Section 5.6). In some embodiments, provided herein is a method for treating fibrosis in a subject in need thereof, comprising administering a pharmaceutical composition (such as a pharmaceutical composition described in Section 5.6). In a specific embodiment, the subject is human.

In some embodiments, an effective amount of the modified cells described in Section 5.5, can be combined with one or more agents. In some embodiments, the agent is capable of preventing or reducing interaction between CD47 and SIRPα (see, e.g., WO 2019/241403, incorporated by reference in its entirety). In some embodiments, the agent comprises an anti-CD47 antibody. Non-limiting examples of suitable anti-CD47 antibodies include clones B6H12, 5F9, 8B6, and C3 (see, e.g., WO 2011/143624, incorporated by reference in its entirety). In some embodiments, the agent comprises a soluble CD47 polypeptide. In some embodiments, the agent comprises an anti-SIRPα antibody. In some embodiments, the agent is capable of preventing or reducing interaction between CD24 and SIGLEC10 (see, e.g., WO 2019/241403, incorporated by reference in its entirety). In some embodiments, the agent comprises an anti-CD24 antibody. In some embodiments, the agent comprises an anti-SIGLEC10 antibody. In some embodiments, the agent comprises a soluble SIGLEC10 polypeptide. In some embodiments, the agent comprises an anti-CD20 antibody (e.g., rituximab). In some embodiments, the agent comprises an anti-HER2/neu antibody (e.g., trastuzumab). In certain embodiments, the modified cell of the present disclosure is precomplexed with one or more agents.

### 5.8. Methods of Engineering a Modified Cell

Also provided herein, is a method of engineering a modified cell, comprising introducing into a cell (a) a polynucleotide encoding a CAR (such as a polynucleotide described in Section 5.2), (b) a vector comprising a polynucleotide encoding a CAR (such as a vector described in Section 5.3), or (c) a CAR polypeptide (such as a polypeptide described in Section 5.4). In some embodiments, the method further comprises, selecting a cell for expression of the CAR. In some embodiments, the cell is a monocyte, a macrophage, or an induced pluripotent stem cell (iPSC). In some embodiments, the cell is an iPSC. In some embodiments, the method further comprises differentiating the iPSC into a monocyte or macrophage. In some embodiments, the method further comprises differentiating the iPSC into a monocyte. In some embodiments, the method further comprises differentiating the iPSC into a macrophage. In some embodiments, the cell is a macrophage. In some embodiments, the macrophage is derived from an iPSC. In some embodiments, the cell is a monocyte. In some embodiments, the monocyte is derived from an iPSC. In some embodiments, the method further comprises differentiating the monocyte into a macrophage. In some embodiments, the differentiation of the monocyte into a macrophage is performed in vitro, ex vivo, or in vivo. In some embodiments, the cell is a human cell. In some embodiments, the cells provided herein comprise a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the cells provided herein comprise genetic disruption of SIRPA. In some embodiments, the cells provided herein comprise genetic disruption of CISH. In some embodiments, the cells provided herein comprise genetic disruption of SIGLEC10. In specific embodiments, the cells provided herein comprise genetic disruption of SIRPA and CISH. In certain embodiments, the cells provided herein comprise genetic disruption of SIRPA and SIGLEC10. In further embodiments, the cells provided herein comprise genetic disruption of SIGLEC10 and CISH. In still further embodiments, the cells provided herein comprise genetic disruption of SIGLEC10, CISH, and SIRPA.

In some embodiments, provided herein is a method of engineering a modified cell, comprising introducing into a cell a polynucleotide encoding a CAR (such as a polynucleotide described in Section 5.2). In some embodiments, the method further comprises, selecting a cell for expression of the CAR. In some embodiments, the cell is a monocyte, a macrophage, or an induced pluripotent stem cell (iPSC). In some embodiments, the cell is an iPSC. In some embodiments, the method further comprises differentiating the iPSC into a monocyte or macrophage. In some embodiments, the method further comprises differentiating the iPSC into a monocyte. In some embodiments, the method further comprises differentiating the iPSC into a macrophage. In some embodiments, the cell is a macrophage. In some embodiments, the macrophage is derived from an iPSC. In some embodiments, the cell is a monocyte. In some embodiments, the monocyte is derived from an iPSC. In some embodiments, the method further comprises differentiating the monocyte into a macrophage. In some embodiments, the differentiation of the monocyte into a macrophage is performed in vitro, ex vivo, or in vivo. In some embodiments, the cell is a human cell. In some embodiments the cells provided herein comprise a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene (such as a modified cell described in Section 5.5). In some embodiments, the cells provided herein comprise genetic disruption of SIRPA. In some embodiments, the cells provided herein comprise genetic disruption of CISH. In some embodiments, the cells provided herein comprise genetic disruption of SIGLEC10. In specific embodiments, the cells provided herein comprise genetic disruption of SIRPA and CISH. In certain embodiments, the cells provided herein comprise genetic disruption of SIRPA and SIGLEC10. In further embodiments, the cells provided herein comprise genetic disruption of SIGLEC10 and CISH. In still further embodiments, the cells provided herein comprise genetic disruption of SIGLEC10, CISH, and SIRPA. In certain embodiments, the method comprises integrating a polynucleotide encoding the CAR (such as a polynucleotide described in Section 5.2) into a safe harbor locus, e.g., an AAVS1 locus. In certain embodiments, the method comprises integrating a polynucleotide encoding the CAR (such as a polynucleotide described in Section 5.4) into a target gene selected from the group consisting of a SIRPA gene, a CISH gene, and a SIGLEC10 gene, such that the target gene(s) is genetically disrupted.

In some embodiments, provided herein is a method of engineering a modified cell, comprising introducing into the cell a vector comprising a polynucleotide encoding a CAR (such as a vector described in Section 5.3. In some embodiments, the method further comprises, selecting a cell for expression of the CAR. In some embodiments, the cell is a monocyte, a macrophage, or an induced pluripotent stem cell (iPSC). In some embodiments, the cell is an iPSC. In some embodiments, the method further comprises differentiating the iPSC into a monocyte or macrophage. In some embodiments, the method further comprises differentiating the iPSC into a monocyte. In some embodiments, the method further comprises differentiating the iPSC into a macrophage. In some embodiments, the cell is a macrophage. In some embodiments, the macrophage is derived from an iPSC. In some embodiments, the cell is a monocyte. In some embodiments, the monocyte is derived from an iPSC. In some embodiments, the method further comprises differentiating the monocyte into a macrophage. In some embodiments, the differentiation of the monocyte into a macrophage is performed in vitro, ex vivo, or in vivo. In some embodiments, the cell is a human cell. In some embodiments the cells provided herein comprise a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene (such as a modified cell described in Section 5.5). In some embodiments, the cells provided herein comprise genetic disruption of SIRPA. In some embodiments, the cells provided herein comprise genetic disruption of CISH. In some embodiments, the cells provided herein comprise genetic disruption of SIGLEC10. In specific embodiments, the cells provided herein comprise genetic disruption of SIRPA and CISH. In certain embodiments, the cells provided herein comprise genetic disruption of SIRPA and SIGLEC10. In further embodiments, the cells provided herein comprise genetic disruption of SIGLEC10 and CISH. In still further embodiments, the cells provided herein comprise genetic disruption of SIGLEC10, CISH, and SIRPA.

In some embodiments, provided herein is a method of engineering a modified cell, comprising introducing into a cell a CAR polypeptide (such as a polypeptide described in Section 5.4). In some embodiments, the method further comprises, selecting a cell for expression of the CAR. In some embodiments, the cell is a monocyte, a macrophage, or an induced pluripotent stem cell (iPSC). In some embodiments, the cell is an iPSC. In some embodiments, the method further comprises differentiating the iPSC into a monocyte or macrophage. In some embodiments, the method further comprises differentiating the iPSC into a monocyte. In some embodiments, the method further comprises differentiating the iPSC into a macrophage. In some embodiments, the cell is a macrophage. In some embodiments, the macrophage is derived from an iPSC. In some embodiments, the cell is a monocyte. In some embodiments, the monocyte is derived from an iPSC. In some embodiments, the method further comprises differentiating the monocyte into a macrophage. In some embodiments, the differentiation of the monocyte into a macrophage is performed in vitro, ex vivo, or in vivo. In some embodiments, the cell is a human cell. In some embodiments the cells provided herein comprise a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene (such as a modified cell described in Section 5.5). In some embodiments, the cells provided herein comprise genetic disruption of SIRPA. In some embodiments, the cells provided herein comprise genetic disruption of CISH. In some embodiments, the cells provided herein comprise genetic disruption of SIGLEC10. In specific embodiments, the cells provided herein comprise genetic disruption of SIRPA and CISH. In certain embodiments, the cells provided herein comprise genetic disruption of SIRPA and SIGLEC10. In further embodiments, the cells provided herein comprise genetic disruption of SIGLEC10 and CISH. In still further embodiments, the cells provided herein comprise genetic disruption of SIGLEC10, CISH, and SIRPA.

### 5.9. Assays

### 5.9.1. Assays for Measuring Cytokine Secretion

Any assay known in the art for measuring cytokine secretion from a cell can be used. Non-limiting examples include immunoassays (e.g., enzyme-linked immunosorbent assay (ELISA), or radioimmunoprecipitation assay (RIP)), and cell-based in vitro bioassays, and may depend on the type of cytokine seeking detection. For example, the cytokine of interest can be immobilized to a microtiter plate, and a test sample (e.g., a cell culture supernatant sample from a cultured macrophage) can be added to the wells of the plate. If the test sample contains a detectable amount of the cytokine, detection can be achieved using a detection antibody (e.g., anti-human/mouse/rabbit IgG, IgM, IgA, or IgE HRP conjugate, anti-human/mouse/rabbit Fc HRP conjugate). One exemplary assay for measuring secretion of a cytokine (e.g., a pro-inflammatory cytokine) involves measuring the cytokine by ELISA after culturing a cell (e.g., a macrophage) for about 24 hours in the presence of a target antigen. Other conventional methods can also be employed as suitable.

### 5.9.2. Assays for Measuring Phagocytosis

Any assay known in the art for measuring phagocytosis can be used. Non-limiting examples include flow cytometry quantification of phagocytosis, and fluorescent microscopy that employs a pH-sensitive dye. The phagocytosis assay could be performed with bacterial particles or a stably transformed cell line. For example, an exemplary assays involves measuring phagocytosis by co-incubation of a macrophage (e.g., a macrophage expressing a CAR) labeled with a fluorescent reporter (e.g., RFP) and a target cell expressing a target antigen that is labeled with a different fluorescent reporter (e.g., GFP) for a suitable period of time (e.g., about four hours), and measuring the amount of double positive cells (RFP+ and GFP+) by flow cytometry. Phagocytosis Index (PI) was calculated as follows:

$$PI = 100 \times = \frac{\% Double Positive Cells}{\% Double Positive Cells + \% GFP only Cells} .$$

Other conventional methods can also be employed as suitable.

### 5.9.3. Assays for Measuring Promoter Activity

Any assay known in the art for measuring promoter activity in a cell can be used. Non-limiting examples include measuring expression of a reporter gene (e.g., luciferase, or any fluorescence protein, such as GFP, RFP, or YFP) from the promoter of the gene of interest, including fluorescence proteins. For example, an in vitro luciferase activity can be used assayed to determine promoter activity in transiently transfected human THP-1 cells. A ubiquitous/constitutively active promoter, such as the cytomegalovirus (CMV) basic promoter, can be used as a promoter control, and PGL3-p47-86 can be used as a basal activity control. Other conventional methods can also be employed as suitable.

Comparison between promoter activity in a myeloid cell (e.g., monocyte or macrophage) and a non-myeloid reference cell can also be performed. A representative non-myeloid reference cell may be, for example, a human intestinal epithelial cell (e.g., Caco-2), a cervix epithelioid carcinoma cell (e.g., HeLa), a human embryonic kidney cell 293 (e.g., HEK-293 or 293T), a T lymphocyte (e.g., Jurkat), or a mouse osteoblast (e.g., Oct-1).

### 6. EMBODIMENTS

This invention provides the following non-limiting embodiments.

A1. A polynucleotide encoding a chimeric antigen receptor (CAR), wherein the CAR comprises an antigen recognition moiety, a hinge domain, a transmembrane domain, and an intracellular domain comprising:
(a) a non-lymphoid intracellular signaling domain; and
(b) a CD3ζ intracellular signaling domain, and
wherein the polynucleotide is operatively linked to a promoter.

A2. The polynucleotide of embodiment A1, wherein the promoter is a myeloid-specific promoter.

A3. The polynucleotide of embodiment A2, wherein the myeloid-specific promoter is a native macrophage or a native monocyte promoter.

A4. The polynucleotide of embodiment A2, wherein the myeloid-specific promoter is a synthetic promoter.

A5. The polynucleotide of embodiment A4, wherein the synthetic promoter is selected from the group consisting of synthetic promoter-146 (SP146), synthetic promoter-107 (SP107), and synthetic promoter-60 (SP60).

A6. The polynucleotide of embodiment A1, wherein the promoter is selected from the group consisting of a EF1α, CD36, CD68, SP60, SP107, SP146, CAG, PGK, T7 and CMV promoter.

A7. The polynucleotide of any one of embodiments A1 to A6, wherein the antigen recognition moiety comprises a scFv antigen recognition moiety.

A8. The polynucleotide of embodiment A7, wherein the scFv antigen recognition moiety recognizes a tumor antigen or fragment thereof.

A9. The polynucleotide of embodiment A8, wherein the tumor antigen is selected from the group consisting of HER2/neu, PSMA, Claudin18, CD20, CD5, mesothelin, GD2, and CD19.

A10. The polynucleotide of any one of embodiments A1 to A9, wherein the hinge domain comprises a CD8 hinge domain.

A11. The polynucleotide of any one of embodiments A1 to A10, wherein the transmembrane domain enhances activity of a CAR in a macrophage relative to the CAR having a transmembrane domain consisting of a CD8 transmembrane domain.

A12. The polynucleotide of embodiment A11, wherein the increase in activity comprises an increase in secretion of a pro-inflammatory cytokine.

A13. The polynucleotide of embodiment A12, wherein the increase in secretion of the pro-inflammatory cytokine is about 10-fold to about 100-fold.

A14. The polynucleotide of embodiment A13, wherein the increase in secretion of the pro-inflammatory cytokine is about 25-fold.

A15. The polynucleotide of embodiment A13, wherein the increase in secretion of the pro-inflammatory cytokine is about 50-fold.

A16. The polynucleotide of embodiment A13, wherein the increase in secretion of the pro-inflammatory cytokine is about 75-fold.

A17. The polynucleotide of embodiment A13, wherein the increase in secretion of the pro-inflammatory cytokine is about 100-fold.

A18. The polynucleotide of embodiment A13, wherein the increase in secretion of the pro-inflammatory cytokine is more than about 100-fold.

A19. The polynucleotide of embodiment A12, wherein the pro-inflammatory cytokine is selected from the group consisting of CXCL10, CCL1, CCL2, CCL22, MIP-1α/CCL3, IL-10, IL-1β, IFNγ, IL-6, TNFA, and CCL20, or a combination thereof.

A20. The polynucleotide of embodiment A12, wherein the pro-inflammatory cytokine comprises MIP-1α/CCL3.

A21. The polynucleotide of embodiment A19, wherein the increase in secretion of the pro-inflammatory cytokine comprises a composite score of two of more cytokines.

A22. The polynucleotide of any one of embodiments A12 to A21, wherein the secretion of the pro-inflammatory cytokine is measured by ELISA after culturing the macrophage for 24 hours in the presence of a target antigen.

A23. The polynucleotide of any one of embodiments All to A22, wherein the increase in activity comprises an increase in macrophage phagocytosis.

A24. The polynucleotide of embodiment A23, wherein the increase in macrophage phagocytosis is about 10% to about 50%.

A25. The polynucleotide of embodiment A24, wherein the increase is about 10%.

A26. The polynucleotide of embodiment A24, wherein the increase is about 20%.

A27. The polynucleotide of embodiment A24, wherein the increase is about 30%.

A28. The polynucleotide of embodiment A24, wherein the increase is about 40%.

A29. The polynucleotide of embodiment A24, wherein the increase is more than about 40%.

A30. The polynucleotide of any one of embodiments A23 to A29, wherein phagocytosis is measured by flow cytometry after about four hours of co-incubation of a macrophage expressing the CAR and a target cell expressing a target antigen.

A31. The polynucleotide of any one of embodiments A1 to A30, wherein the transmembrane domain is selected from the group consisting of IL-1R1, VEGFR2, IFNγR1, CSF3R, CSF2R, SLAMF7, DECTIN-1, DECTIN-3, TLR4, CSF1R, CD8, CD28, MINCLE, MDL1, BDCA2, DAP12, CD11b, CD86, and CD40.

A32. The polynucleotide of any one of embodiments A1 to A31, wherein the intracellular domain increases activity of a CAR in a macrophage relative to the CAR having an intracellular domain consisting of a CD3ζ intracellular signaling domain.

A33. The polynucleotide of embodiment A32, wherein the increase in activity comprises an increase in secretion of a pro-inflammatory cytokine.

A34. The polynucleotide of embodiment A33, wherein the increase in secretion of the pro-inflammatory cytokine is about 10-fold to about 100-fold.

A35. The polynucleotide of embodiment A33, wherein the increase in secretion of the pro-inflammatory cytokine is about 25-fold.

A36. The polynucleotide of embodiment A33, wherein the increase in secretion of the pro-inflammatory cytokine is about 50-fold.

A37. The polynucleotide of embodiment A33, wherein the increase in secretion of the pro-inflammatory cytokine is about 75-fold.

A38. The polynucleotide of embodiment A33, wherein the increase in secretion of the pro-inflammatory cytokine is about 100-fold.

A39. The polynucleotide of embodiment A33, wherein the increase in secretion of the pro-inflammatory cytokine is more than about 100-fold.

A40. The polynucleotide of embodiment A33, wherein the pro-inflammatory cytokine is selected from the group consisting of CXCL10, CCL1, CCL2, CCL22, MIP-1α/CCL3, IL-10, IL-1β, IFNγ, IL-6, TNFA, and CCL20, or a combination thereof.

A41. The polynucleotide of embodiment A33, wherein the pro-inflammatory cytokine comprises MIP-1α/CCL3.

A42. The polynucleotide of embodiment A40, wherein the increase in secretion of the pro-inflammatory cytokine comprises a composite score of two of more pro-inflammatory cytokines.

A43. The polynucleotide of any one of embodiments A33 to A42, wherein the secretion of the pro-inflammatory cytokine is measured by ELISA after culturing the macrophage for 24 hours in the presence of a target antigen.

A44. The polynucleotide of any one of embodiments A32 to A43, wherein the increase in activity comprises an increase in macrophage phagocytosis.

A45. The polynucleotide of embodiment A44, wherein the increase in macrophage phagocytosis is about 10% to about 50%.

A46. The polynucleotide of embodiment A45, wherein the increase is about 10%.

A47. The polynucleotide of embodiment A45, wherein the increase is about 20%.

A48. The polynucleotide of embodiment A45, wherein the increase is about 30%.

A49. The polynucleotide of embodiment A45, wherein the increase is about 40%.

A50. The polynucleotide of embodiment A45, wherein the increase is more than about 40%.

A51. The polynucleotide of any one of embodiments A44 to A50, wherein phagocytosis is measured by flow cytometry after about four hours of co-incubation of a macrophage expressing the CAR and a target cell expressing a target antigen.

A52. The polynucleotide of any one of embodiments A1 to A51, wherein the one or more non-lymphoid intracellular signaling domains are selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, and CD80/B7-1.

A53. A polynucleotide encoding a chimeric antigen receptor (CAR), wherein the CAR comprises
(a) an antigen recognition moiety;
(b) a CD8 hinge domain,
(c) a transmembrane domain, wherein the transmembrane domain is selected from the group consisting of
   (i) a CD8 transmembrane domain;
   (ii) a SLAMF7 transmembrane domain;
   (iii) a CD86 transmembrane domain;
   (iv) a DECTIN-1 transmembrane domain; and
   (v) a CSF1R transmembrane domain;
(d) a first intracellular signaling domain, wherein the first intracellular signaling domain is selected from the group consisting of
   (i) a CD86 intracellular signaling domain;
   (ii) a Lox1c intracellular signaling domain,
   (iii) a 2B4 intracellular signaling domain;
   (iv) a DECTIN-1 intracellular signaling domain; and
   (v) a CD40 intracellular signaling domain; and
(e) a second intracellular domain comprising a CD3ζ intracellular signaling domain,
wherein the polynucleotide is operatively linked to a promoter.

A54. The polynucleotide of embodiment A53, wherein the polynucleotide encodes for a hinge domain comprising an amino acid sequence of SEQ ID NO:1.

A55. The polynucleotide of embodiment A54, wherein the transmembrane domain comprises a CD8 transmembrane domain.

A56. The polynucleotide of embodiment A55, wherein the first intracellular signaling domain comprises a CD86 intracellular signaling domain.

A57. The polynucleotide of embodiment A55, wherein the first intracellular signaling domain comprises a Lox1c intracellular signaling domain.

A58. The polynucleotide of embodiment A54, wherein the transmembrane domain comprises a SLAMF7 transmembrane domain.

A59. The polynucleotide of embodiment A58, wherein the first intracellular signaling domain comprises a CD86 intracellular signaling domain.

A60. The polynucleotide of embodiment A54, wherein the transmembrane domain comprises a CD86 transmembrane domain.

A61. The polynucleotide of embodiment A60, the first intracellular signaling domain comprises a 2B4 intracellular signaling domain.

A62. The polynucleotide of embodiment A54, wherein the transmembrane domain comprises a DECTIN-1 transmembrane domain.

A63. The polynucleotide of embodiment A54, wherein the transmembrane domain comprises a CSF1R transmembrane domain.

A64. The polynucleotide of embodiment A62, wherein the first intracellular signaling domain comprises a DECTIN-1 intracellular signaling domain.

A65. The polynucleotide of embodiment A55, wherein the first intracellular signaling domain comprises a CD40 intracellular signaling domain.

A66. The polynucleotide of embodiment A53, wherein the polynucleotide encodes for a hinge domain comprising an amino acid sequence of SEQ ID NO:4.

A67. The polynucleotide of embodiment A66, wherein the first intracellular signaling domain comprises a CD86 intracellular signaling domain.

A68. The polynucleotide of embodiment A67, wherein the transmembrane domain comprises a SLAMF7 transmembrane domain.

A69. The polynucleotide of any one of embodiments A53 to A68, wherein the promoter is a constitutive promoter.

A70. The polynucleotide of embodiment A69, wherein the constitutive promoter is a EF1α promoter, a CAG promoter, or a T7 promoter.

A71. The polynucleotide of any one of embodiments A53 to A68, wherein the promoter is a myeloid-specific promoter.

A72. The polynucleotide of embodiment A71, wherein the myeloid-specific promoter is a native macrophage promoter or a native monocyte promoter.

A73. The polynucleotide of embodiment A71, wherein the myeloid-specific promoter is a synthetic promoter.

A74. The polynucleotide of embodiment A73, wherein the synthetic promoter is selected from the group consisting of synthetic promoter-146 (SP146), synthetic promoter-107 (SP107), and synthetic promoter-60 (SP60).

A75. The polynucleotide of embodiment A74, wherein the synthetic promoter is SP146.

A76. The polynucleotide of any one of embodiments A53 to A75, wherein the antigen recognition moiety comprises a scFv antigen recognition moiety.

A77. The polynucleotide of embodiment A76, wherein the scFv antigen recognition moiety recognizes a tumor antigen or fragment thereof.

A78. The polynucleotide of embodiment A77, wherein the tumor antigen is selected from your group consisting of HER2/neu, PSMA, Claudin18, CD20, CD5, mesothelin, GD2, and CD19.

A79. A vector comprising the polynucleotide of any one of embodiments A1 to A78.

A80. The vector of embodiment A79, wherein the vector comprises DNA or RNA.

A81. The vector of embodiment A79 or A80, wherein the vector is a plasmid vector.

A82. The vector of embodiment A79 or A80, wherein the vector is a viral vector.

A83. The vector of embodiment A82, wherein the viral vector is selected from the group consisting of an adenoviral vector, a lentiviral vector, and a retroviral vector.

A84. The vector of embodiment A82, wherein the viral vector is a lentiviral vector.

A85. A polypeptide comprising:
(a) an antigen recognition moiety polypeptide;
(b) a CD8 hinge domain polypeptide;
(c) a transmembrane domain polypeptide, wherein the transmembrane domain is selected from the group consisting of
   (i) a CD8 transmembrane domain;
   (ii) a SLAMF7 transmembrane domain;
   (iii) a CD86 transmembrane domain;
   (iv) a DECTIN-1 transmembrane domain; and
   (v) a CSF1R transmembrane domain;
(d) a first intracellular signaling domain polypeptide, wherein the first intracellular signaling domain is selected from the group consisting of:
   (i) a CD86 intracellular signaling domain;
   (ii) a Lox1c intracellular signaling domain;
   (iii) a 2B4 intracellular signaling domain;
   (iv) a DECTIN-1 intracellular signaling domain; and
   (v) a CD40 intracellular signaling domain; and
(e) a second intracellular domain polypeptide comprising a CD3ζ intracellular signaling domain.

A86. The polypeptide of embodiment A85, wherein the hinge domain has an amino acid sequence of SEQ ID NO:1.

A87. The polypeptide of embodiment A86, wherein the transmembrane domain polypeptide comprises a CD8 transmembrane domain polypeptide.

A88. The polypeptide of embodiment A87, wherein the CD8 transmembrane domain polypeptide has an amino acid sequence of SEQ ID NO:11.

A89. The polypeptide of embodiment A87 or A88, wherein the first intracellular signaling domain polypeptide comprises a CD86 intracellular signaling domain polypeptide.

A90. The polypeptide of embodiment A89, wherein the CD86 intracellular signaling domain polypeptide has an amino acid sequence of SEQ ID NO:40.

A91. The polypeptide of embodiment A87 or A88, wherein the first intracellular signaling domain polypeptide comprises a Lox1c intracellular signaling domain polypeptide.

A92. The polypeptide of embodiment A91, wherein the Lox1c intracellular signaling domain polypeptide has an amino acid sequence of SEQ ID NO:41.

A93. The polypeptide of embodiment A86, wherein the transmembrane domain polypeptide comprises a SLAMF7 transmembrane domain polypeptide.

A94. The polypeptide of embodiment A93, wherein the SLAMF7 transmembrane domain polypeptide has an amino acid sequence of SEQ ID NO:12.

A95. The polypeptide of embodiment A93 or A94, wherein the first intracellular signaling domain polypeptide comprises a CD86 intracellular signaling domain polypeptide.

A96. The polypeptide of embodiment A95, wherein the CD86 intracellular signaling domain polypeptide has an amino acid sequence of SEQ ID NO:40.

A97. The polypeptide of embodiment A86, wherein the transmembrane domain polypeptide comprises a CD86 transmembrane domain polypeptide.

A98. The polypeptide of embodiment A97, wherein the CD86 transmembrane domain polypeptide has an amino acid sequence of SEQ ID NO:13.

A99. The polypeptide of embodiment A97 or A98, wherein the first intracellular signaling domain polypeptide comprises a 2B4 intracellular signaling domain polypeptide.

A100. The polypeptide of embodiment A99, wherein the 2B4 intracellular signaling domain polypeptide has an amino acid sequence of SEQ ID NO:42.

A101. The polypeptide of embodiment A86, wherein the transmembrane domain polypeptide comprises a DECTIN-1 transmembrane domain polypeptide.

A102. The polypeptide of embodiment A101, wherein the DECTIN-1 transmembrane domain polypeptide has an amino acid sequence of SEQ ID NO: 14.

A103. The polypeptide of embodiment A101 or A102, wherein the first intracellular signaling domain polypeptide comprises a DECTIN-1 intracellular signaling domain polypeptide.

A104. The polypeptide of embodiment A103, wherein the DECTIN-1 intracellular signaling domain polypeptide has an amino acid sequence of SEQ ID NO:43.

A105. The polypeptide of embodiment A85, wherein the hinge domain polypeptide comprises SEQ ID NO:4.

A106. The polypeptide of embodiment A105, wherein the transmembrane domain polypeptide comprises a SLAMF7 transmembrane domain.

A107. The polypeptide of embodiment A106, the SLAMF7 transmembrane domain polypeptide has an amino acid sequence of SEQ ID NO: 12.

A108. The polypeptide of embodiment A106 or A107, wherein the first intracellular signaling domain polypeptide comprises a CD86 intracellular signaling domain polypeptide.

A109. The polypeptide of embodiment A108, wherein the CD86 intracellular signaling domain polypeptide has an amino acid sequence of SEQ ID NO:40.

A110. The polypeptide of any one of embodiments to A85 to A109, wherein the antigen recognition moiety polypeptide comprises a scFv antigen recognition moiety polypeptide.

A111. The polypeptide of embodiment A110, wherein the scFv antigen recognition moiety polypeptide recognizes a tumor antigen or fragment thereof.

A112. The polynucleotide of embodiment A111, wherein the tumor antigen is selected from your group consisting of HER2/neu, PSMA, Claudin18, CD20, CD5, mesothelin, GD2, and CD19.

A113. A polypeptide having an amino acid sequence comprising or consisting of SEQ ID NO: 97.

A114. A polypeptide having an amino acid sequence comprising or consisting of SEQ ID NO: 98.

A115. A polypeptide having an amino acid sequence comprising or consisting of SEQ ID NO: 99.

A116. A polypeptide having an amino acid sequence comprising or consisting of SEQ ID NO: 100.

A117. A polypeptide having an amino acid sequence comprising or consisting of SEQ ID NO: 101.

A118. A polypeptide having an amino acid sequence comprising or consisting of SEQ ID NO: 102.

A119. A polypeptide having an amino acid sequence comprising or consisting of SEQ ID NO: 103.

A120. A polypeptide having an amino acid sequence comprising or consisting of SEQ ID NO: 104.

A121. A polypeptide having an amino acid sequence comprising or consisting of SEQ ID NO: 105.

A122. A polypeptide having an amino acid sequence comprising or consisting of SEQ ID NO: 106.

A123. A polypeptide having an amino acid sequence comprising or consisting of SEQ ID NO: 107.

A124. A polypeptide having an amino acid sequence comprising or consisting of SEQ ID NO: 108.

A125. A polypeptide having an amino acid sequence comprising or consisting of SEQ ID NO: 110.

A126. A polypeptide having an amino acid sequence comprising or consisting of SEQ ID NO: 111.

A127. A polypeptide having an amino acid sequence comprising or consisting of SEQ ID NO: 112.

A128. A polypeptide having an amino acid sequence comprising or consisting of SEQ ID NO: 113.

A129. A polypeptide having an amino acid sequence comprising or consisting of SEQ ID NO: 114.

A130. A polypeptide having an amino acid sequence comprising or consisting of SEQ ID NO: 115.

A131. A polypeptide having an amino acid sequence comprising or consisting of SEQ ID NO: 116.

A132. A polypeptide having an amino acid sequence comprising or consisting of SEQ ID NO: 117.

A133. A polypeptide having an amino acid sequence comprising or consisting of SEQ ID NO: 118.

A134. A polypeptide having an amino acid sequence comprising or consisting of SEQ ID NO: 119.

A135. A polypeptide having an amino acid sequence comprising or consisting of SEQ ID NO: 120.

A136. A polypeptide having an amino acid sequence comprising or consisting of SEQ ID NO: 121.

A137. A polypeptide having an amino acid sequence comprising or consisting of SEQ ID NO: 125.

A138. A polypeptide having an amino acid sequence comprising or consisting of SEQ ID NO: 126.

A139. A polypeptide having an amino acid sequence comprising or consisting of SEQ ID NO: 127.

A140. A polypeptide having an amino acid sequence comprising or consisting of SEQ ID NO: 128.

A141. A polypeptide having an amino acid sequence comprising or consisting of SEQ ID NO: 129.

A142. A polypeptide having an amino acid sequence comprising or consisting of SEQ ID NO: 130.

A143. A polypeptide having an amino acid sequence comprising or consisting of SEQ ID NO: 131.

A144. A polypeptide having an amino acid sequence comprising or consisting of SEQ ID NO: 132.

A145. A modified cell, wherein the modified cell comprises the polynucleotide of any one of embodiments A1 to A78, the vector of any one of embodiments A79 to A84, or the polypeptide of any one of embodiments A85 to A144, and wherein the modified cell is a monocyte, a macrophage, or an induced pluripotent stem cell (iPSC).

A146. The modified cell of embodiment A145, wherein the modified cell is an iPSC.

A147. The modified cell of embodiment A145, wherein the modified cell is a macrophage.

A148. The modified cell of embodiment A145, wherein the modified cell is a monocyte.

A149. The modified cell of embodiment A147 or A148, wherein the modified cell is derived from an iPSC.

A150. The modified cell of any one of embodiments A145 to A149, wherein the modified cell is a human cell.

A151. The modified cell of any one of embodiments A145 to A150, comprising a genetic disruption of:
(a) a signal regulatory protein alpha (SIRPA) gene;
(b) a cytokine inducible SH2 containing protein (CISH) gene; and/or
(c) a sialic acid-binding Ig-like lectin 10 (SIGLEC10) gene.

A152. The modified cell of embodiment A151, wherein the modified cell comprises genetic disruption of SIRPA.

A153. The modified cell of embodiment A151 or A152, wherein the modified cell comprises genetic disruption of CISH.

A154. The modified cell of any one of embodiments A151 to A153, wherein the modified cell comprises genetic disruption of SIGLEC10.

A155. The modified cell of any one of embodiments A147 to A154, wherein the modified cell comprises an agent capable of preventing or reducing interaction between CD47 and SIRPα.

A156. The modified cell of embodiment A155, wherein the agent comprises an anti-CD47 antibody.

A157. The modified cell of embodiment A155, wherein the agent comprises an anti-SIRPα antibody.

A158. The modified cell of any one of embodiments A147 to A157, wherein the modified cell comprises an agent capable of preventing or reducing interaction between CD24 and SIGLEC10.

A159. The modified cell of embodiment A158, wherein the agent comprises an anti-CD24 antibody.

A160. The modified cell of embodiment A158, wherein the agent comprises an anti-SIGLEC10 antibody.

A161. A pharmaceutical composition comprising the modified cell of any one of embodiments A147 to A150.

A162. A method of treating a cancer, comprising administering the pharmaceutical composition of embodiment A161.

A163. A method of engineering a modified cell, comprising introducing into the cell the polynucleotide of any one of embodiments A1 to A78, the vector of any one of embodiments A79 to A84, or the polypeptide of any one of embodiments A85 to A144.
wherein the cell is a monocyte, a macrophage, or an induced pluripotent stem cell (iPSC).

A164. The method of embodiment A163, wherein the cell is an iPSC.

A165. The method of embodiment A163, wherein the cell is a macrophage.

A166. The method of embodiment A163, wherein the cell is a monocyte.

A167. The method of embodiment A165 or A166, wherein the cell is derived from an iPSC.

A168. The method of any one of embodiments A163 to A167, wherein the cell is a human cell.

A169. The method of any one of embodiments A163 to A168, wherein the cell comprises a genetic disruption of:
(a) a signal regulatory protein alpha (SIRPA) gene;
(b) a cytokine inducible SH2 containing protein (CISH) gene; and/or
(c) a sialic acid-binding Ig-like lectin 10 (SIGLEC10) gene.

A170. The method of embodiment A169, wherein the modified cell comprises genetic disruption of SIRPA.

A171. The method of embodiment A169 or A170, wherein the modified cell comprises genetic disruption of CISH.

A172. The method of any one of embodiments A169 to A171, wherein the modified cell comprises genetic disruption of SIGLEC10.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations can be within the scope of the following claims.

### 7. EXAMPLES

The examples in this section are offered by way of illustration, and not by way of limitation. The following examples are presented as exemplary embodiments of the invention. They should not be construed as limiting the broad scope of the invention.

### Example 1: Exemplary Assays for Measuring Macrophage Activity

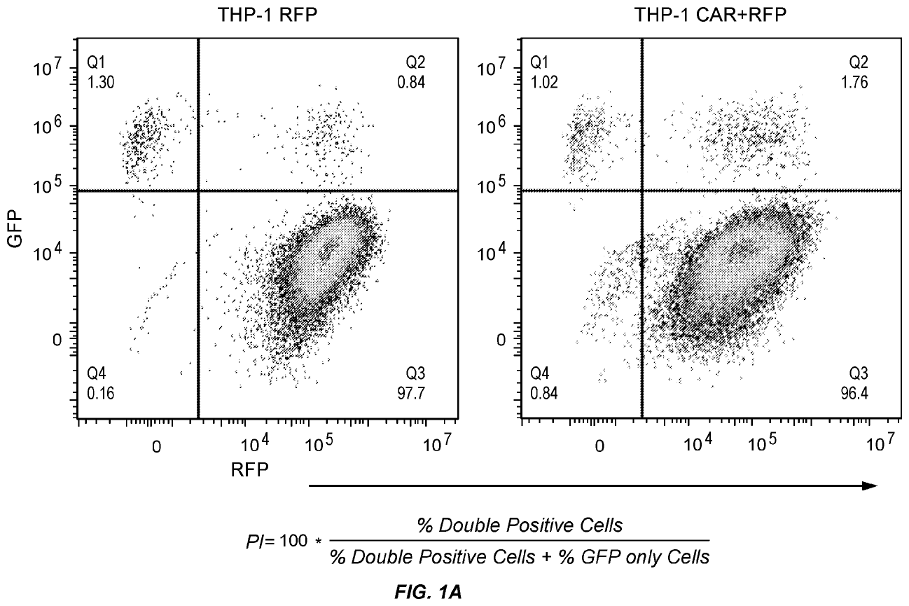

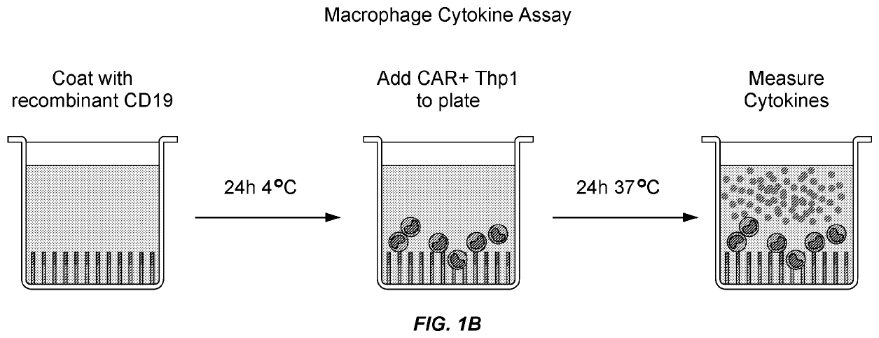

This examples demonstrates that monocytes/macrophages expressing a CAR can be evaluated for macrophage activity using an exemplary phagocytosis assay (FIG. 1A) and/or an exemplary cytokine secretion assay (FIG. 1B).

Briefly, THP-1 monocytes were transduced with lentivirus encoding (1) a CAR, (2) red fluorescence protein (RFP), for detection, and (3) a puromycin resistance gene, for selection. Following transduction, CAR positive cells were enriched by culturing with puromycin. After 2-3 weeks, when the population was 95% RFP positive, macrophage activity was evaluated.

To evaluate phagocytosis, 60,000 stably transduced THP-1 monocytes were seeded per well in a 96-well adherent plate and differentiated into macrophages with 1 nM phorbol 12-myristate-13-acetate (PMA) for 48 hours. After washing PMA out with complete media, 6,000 CD19⁺ target cells were added to each well. The plate was centrifuged for 30 seconds at 300×g and incubated for 4 hours at 37 °C. Supernatant was harvested, then adherent cells were released from the plate using pre-warmed 0.25% Trypsin for 8 minutes and added to the supernatant. Cells were fixed using 37 °C BD Cytofix for 10 minutes, followed by resuspension in staining buffer. Phagocytosis Index (PI) was calculated as follows:

$$PI = 100 \times = \frac{\% Double Positive Cells}{\% Double Positive Cells + \% GFP only Cells} .$$

To evaluate cytokine secretion, tissue culture treated 96-well plates were coated overnight at 4 °C with a recombinant target antigen that corresponded with the CAR antigen recognition moiety. For example, to evaluate cytokine secretion a CAR expressing an scFv that recognized CD19, the tissue culture plates were coated with CD19. The wells were then washed to remove excess antigen before 200,000 stably transduced THP-1 cells were seeded per well and incubated for a further 24 h at 37 °C. The 96-well plate was then centrifuged to pellet the cells and the supernatants was removed for cytokine analysis. Cytokine levels were measured by ELISA assay. To calculate the cytokine index, each raw data value was normalized to the top cytokine producing construct for each tested cytokine in each experiment. The following formula was used: (test CAR/top CAR)×100. The normalized values for each cytokine in the panel were added together to create the index.

Accordingly, monocytes/macrophages expressing a CAR can be evaluated for macrophage activity using an exemplary phagocytosis assay (FIG. 1A) and/or an exemplary cytokine secretion assay (FIG. 1B).

### Example 2: Screening Promoters for Myeloid Specific CAR Expression

This example demonstrates that CAR expression can be enhanced in monocytes/macrophages by use of certain promoters, such as a myeloid specific promoter.

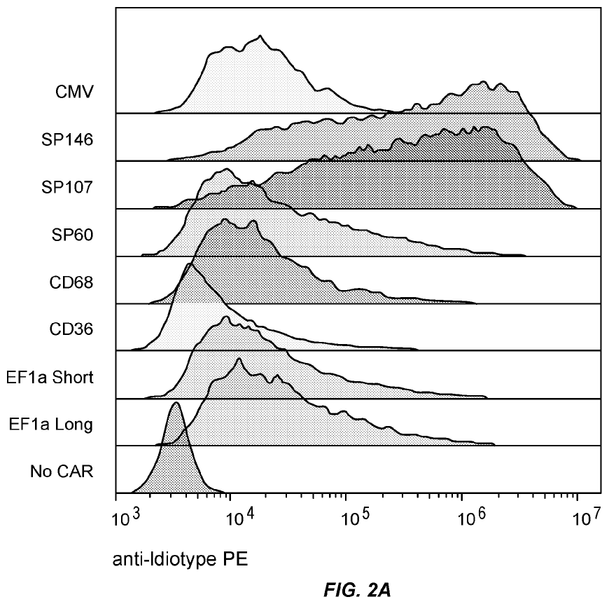

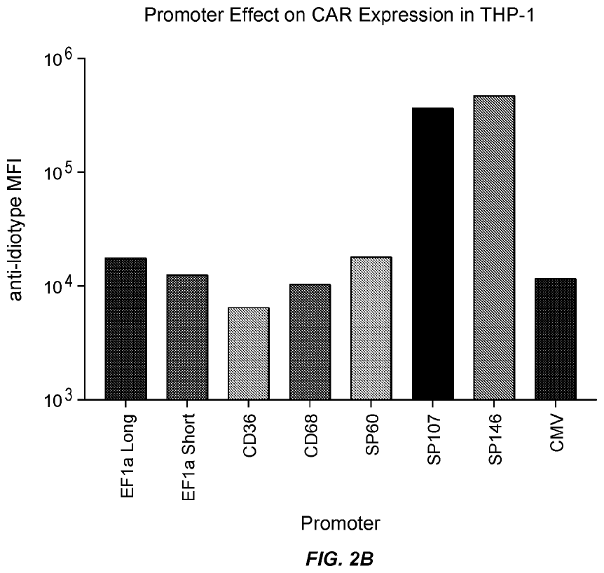

Briefly, a 1^{st} generation CD3ζ CAR was placed under the control of several promoters expected to have enhanced expression in myeloid cells. These CARs were engineered into THP1 cells and stable clones were selected, as described in Example 1. CAR expression was measured by flow cytometry using an anti-idiotype PE antibody (FIG. 2A) against the CAR scFv.

Quantification of the anti-idiotype mean fluorescence intensity (MFI) indicated that CARs under the control of the macrophage-specific synthetic promoters SP107 and SP146 exhibited robust expression, relative to the constitutively active promoters EF1α (long or short) and CMV (FIG. 2B). Expression of the CARs under the control of the macrophage-specific synthetic promoters SP107 and SP146 was also enhanced relative to CARs under the control of proteins that are highly expressed in macrophages, such as promoters from CD36 or CD68 (FIG. 2B).

Taken together, this example demonstrates that promoters that enhance the expression of CARs in myeloid cells can be readily identified, and can be used to express CARs in myeloid cells.

### Example 3: Screening Transmembrane Domains for Enhanced CAR Activity

This example demonstrates that macrophage activity can be enhanced using transmembrane domains from receptors associated with macrophage activity.

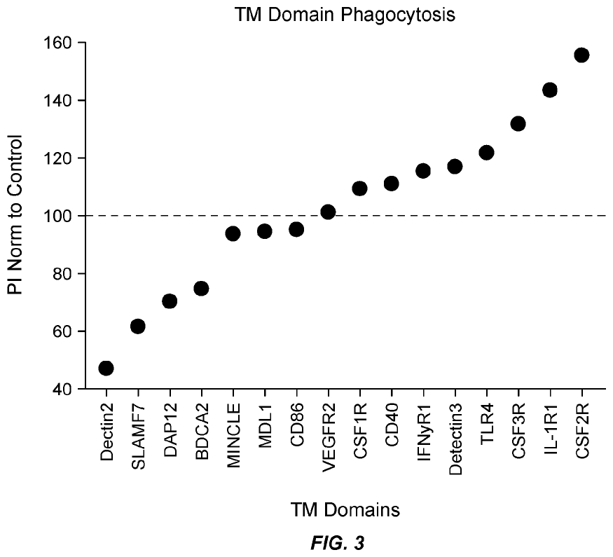

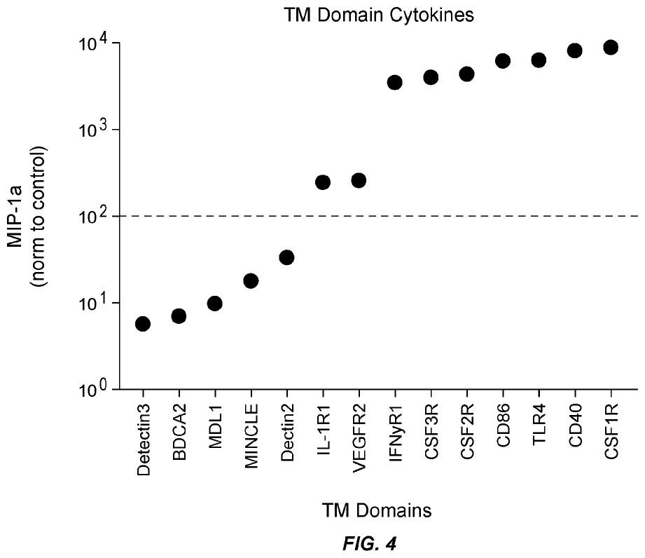

Briefly, the CD8 transmembrane (TM) of a 1^{st} generation CD3ζ CAR was substituted with a series of TM domains from receptors associated with macrophage activity. The effect of these CARs on phagocytosis (FIG. 3) and cytokine production (FIG. 4) was measured in stably transfected THP1 cells, as described in Example 1, and normalized to the parent construct.

The results demonstrated that several of the screened transmembrane domains were able to increase MIP-1α cytokine secretion, relative to the CD8 transmembrane domain, with some achieving nearly 100-fold enhanced secretion levels (FIG. 4). In addition, many of the screened transmembrane domains were also able to increase phagocytosis, relative to the CD8 transmembrane domain, with some achieving about 10% to about 60% increase in phagocytosis (FIG. 3). Interestingly, the CD86 transmembrane domain exhibited a robust improvement in cytokine secretion, and a slight decrease in phagocytosis (FIGS. 3 and 4). Still others, such as the SLAMF7 transmembrane domain, exhibited a significant decrease in phagocytosis (FIG. 3).

Thus, this example demonstrates that certain transmembrane domains can enhance macrophage activity, either by increasing cytokine secretion and/or phagocytosis.

### Example 4: Screening Intracellular Domains for Enhanced CAR Activity

This example demonstrates that macrophage activity can be enhanced by complementing an ITAM containing intracellular domain, such as a CD3ζ intracellular domain, with an additional intracellular domain from a non-lymphoid intracellular signaling domain.

Briefly, the CD3ζ intracellular signaling domain of a 1st generation CD3ζ CAR was complimented with a series of additional IC domains from receptors associated with macrophage activity. The effect of these CARs on phagocytosis and cytokine production was measured in stably transfected THP1 cells, as described in Example 1, and normalized to the parent construct.

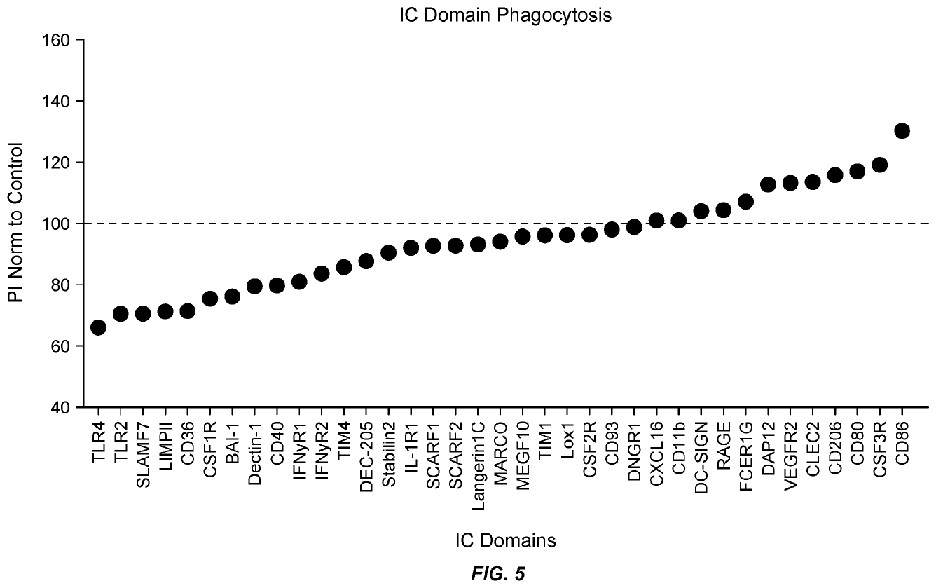

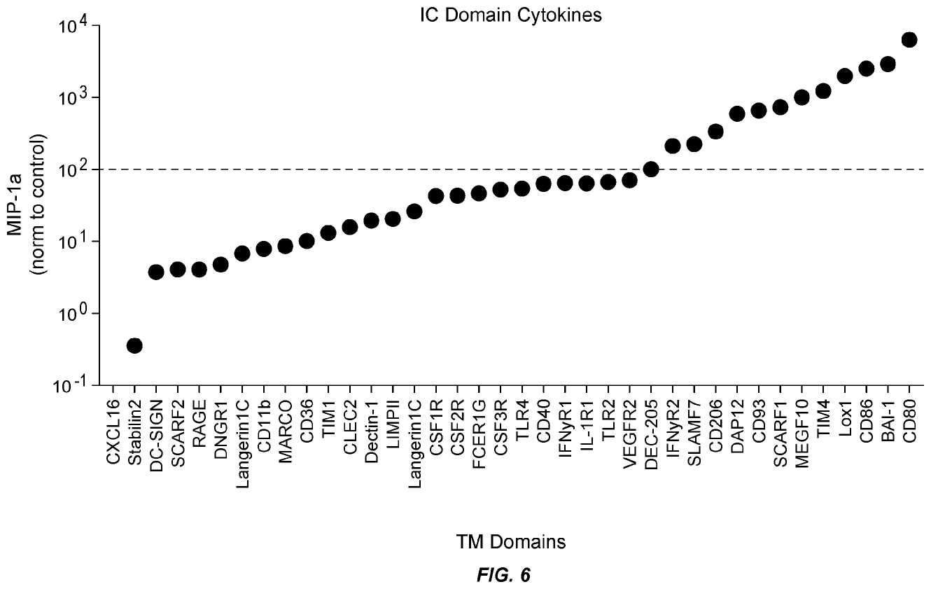

The results demonstrated that several of the screened non-lymphoid intracellular signaling domains were able to increase MIP-1× cytokine secretion when combined with the CD3ζ intracellular signaling domain, relative to the CD3ζ intracellular signaling domain alone, with some achieving 50- to 100-fold enhanced secretion levels (FIG. 6). In addition, several of screened the non-lymphoid intracellular signaling domains were also able to increase phagocytosis when combined with the CD3, intracellular signaling domain, relative to the CD3ζ intracellular signaling domain alone, with some achieving about 10% to about 40% increase in phagocytosis (FIG. 5). Many of the screened non-lymphoid intracellular signaling domains with enhanced cytokine secretion, either had no effect or exhibited decreased phagocytosis, e.g. BAI-1, Lox1, TIM4, MEGF10, CD93, SLAMF7, and IFNγR2 (FIG. 5 and FIG. 6). Further, some of the screened non-lymphoid intracellular signaling domains with enhanced phagocytosis, either had no effect or exhibited decreased cytokine secretion, e.g., CSF3R, VEGFR2, and FCER1G (FIG. 5 and FIG. 6).

Taken together, these data demonstrate that certain intracellular signaling domains can enhance macrophage activity, such as by increasing cytokine secretion and/or phagocytosis.

### Example 5: Macrophage CAR Activity

This example demonstrates that exemplary CARs having an anti-CD19 scFv antigen recognition moiety can be expressed in a monocyte, which is differentiated into a macrophage, and macrophage activity can be evaluated by measuring phagocytosis and/or cytokine section.

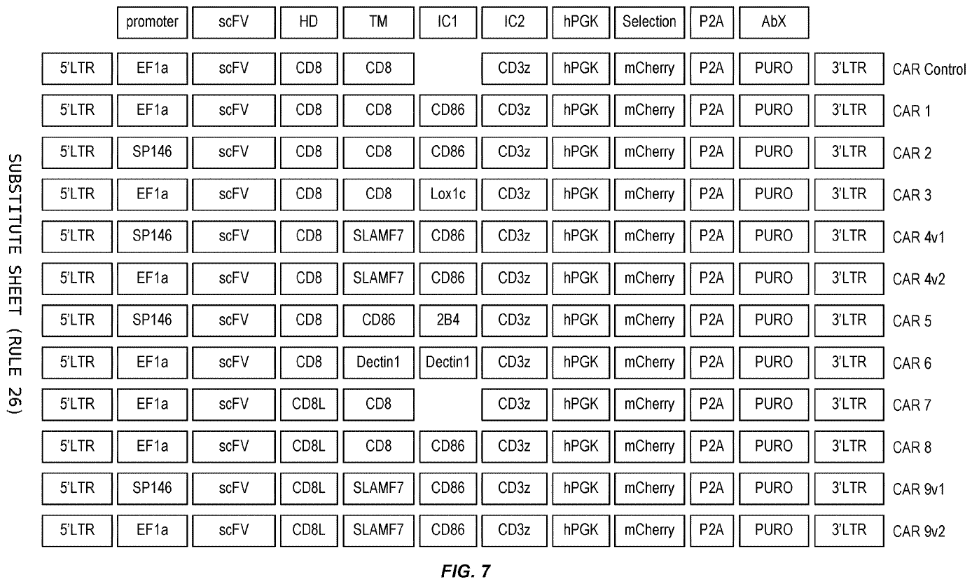

Briefly, THP-1 monocytes were transduced with lentivirus encoding (1) a CAR, (2) red fluorescence protein (RFP), for detection, and (3) a puromycin resistance gene, for selection. Exemplary CARs are illustrated in FIG. 7. Following transduction, CAR positive cells were enriched by culturing with puromycin. After 2-3 weeks, when the population was 95% RFP positive, CAR activity was evaluated as described above in Example 1. Cytokines measured included CXCL10, CCL2, CCL22, CCL3, IL-10, IL-6, TNF&, and CCL20, and a composite score was determined by calculating the sum of all the cytokines, normalized to the secretion levels from a control CAR, which is a first generation CD3ζ CAR. The results from measuring phagocytic index (PI), and/or cytokine secretion are represented in Table 10.

TABLE 10
Macrophage activity for exemplary CARs

| | PI | |
|---|---|---|
| | Normalized | |
| | to control | Cytokine |
| CAR | CAR | Index |
| Control CAR (EF1α/CD8-HD/ | 100 | C1 = 60.06; |
| CD8-TM/CD3ζ-IC) | | C2 = 27.07; |
| | | C3 = 32.73 |
| CAR1 | 130.4 | 208.24 |
| (EF1α/CD8-HD/CD8-TM/CD86- | | (C1 = 60.06) |
| IC1/CD3ζ-IC2) | | |
| CAR2 | 88.50 | 8407.78 |
| (SP146/CD8-HD/CD8-TM/CD86- | | (C3 = 32.73) |
| IC1/CD3ζ-IC2) | | |
| CAR3 | 96.46 | 334.97 |
| (EF1α/CD8-HD/CD8-TM/LOX1C- | | (C2 = 27.07) |
| IC1/CD3ζ-IC2) | | |
| CAR4 | 87.46 | 11954.74 |
| (SP146/CD8-HD/SLAMF7-TM/CD86- | | (C3 = 32.73) |
| IC1/CD3ζ-IC2) | | |
| CAR5 | 93.19 | 13219.89 |
| (SP146/CD8-HD/CD8-TM/2B4- | | (C3 = 32.73) |
| IC1/CD3ζ-IC2) | | |
| CAR6 | 77.82 | 135.48 |
| (EF1α/CD8-HD/DECTIN1-TM/ | | (C2 = 27.07) |
| DECTIN1-IC1/CD3ζ-IC2) | | |
| CAR10 | 117.28 | 225.94 |
| (EF1α/CD8-HD/CD8-TM/CD80- | | (C1 = 60.06) |
| IC1/CD3ζ-IC2) | | |
| CAR11 | 97.33 | 12436.63 |
| (SP146/CD8-HD/SLAMF7-TM/CD80- | | (C3 = 32.73) |
| IC1/CD3ζ-IC2) | | |
| CAR12 | 123.47 | 36.33 |
| (SP146/CD8-HD/CD8-TM/CD80- | | (C3 = 32.73) |
| IC1/CD3ζ-IC2) | | |
| CAR13 | 88.49 | 897.20 |
| (EF1α/CD8L-HD/CD8-TM/CD3ζ-IC) | | (C2 = 27.07) |

Abbreviations:
HD = Hinge Domain;
TM = Transmembrane Domain;
IC = Intracellular Domain

As shown in Table 10, three sets of experiments were performed. The cytokine index for the control CAR for all three sets is listed. For each of the test CARs, the cytokine index of the control CAR used in that set of experiment is indicated below the cytokine index of the test CAR.

As shown in Table 10, the addition of a transmembrane domain and/or intracellular domain derived from a receptor with macrophage activity was able to enhance the activity of the macrophage, as measured by phagocytosis index and/or a cytokine secretion composite score. Notably, for some constructs, the phagocytosis index decreased, whereas the cytokine secretion was significantly increased.

Taken together, this example demonstrates that CARs designed to activate macrophages can be successfully expressed in a monocyte/macrophage, and enhance macrophage activity by increasing phagocytosis and/or cytokine secretion.

### Example 6: Genetic Disruption of iPSCs

This example demonstrates that iPSCs can be genetically disrupted using CRISPR/MAD7, and successfully differentiated into a monocyte.

Briefly, iPSCs were contacted with MAD7 and sgRNA targeting exon 2 of SIRPA (5′-CGACCUCUCUGAUCCCUGUG-3′) that targets the genetic sequence 5′-TGACCTCCCTGATCCCTGTG-3′. Homozygous clones were identified by sequencing. Exemplary clones indicated an insertion (C>CT) within the target exon (Table 11).

TABLE 11
SIRPα KO clone sequencing data
VARIANT SUMMARY

| | | | | SIRPα KO | | SIRPα KO | |
|---|---|---|---|---|---|---|---|
| | | TC1133 | | Clone 1 | | Clone 2 | |
| Target | Variant | Reads | % | Reads | % | Reads | % |
| SIRPα | Del | 0 | 0.00% | 0 | 0.00% | 0 | 0.00% |
| Exon 2 | | | | | | | |
| SIRPα | Ins | 0 | 0.00% | 59233 | 98.24% | 102329 | 97.49% |
| Exon 2 | | | | | | | |
| SIRPα | Coverage | 70412 | NA | 60297 | NA | 104968 | NA |
| Exon 2 | | | | | | | |

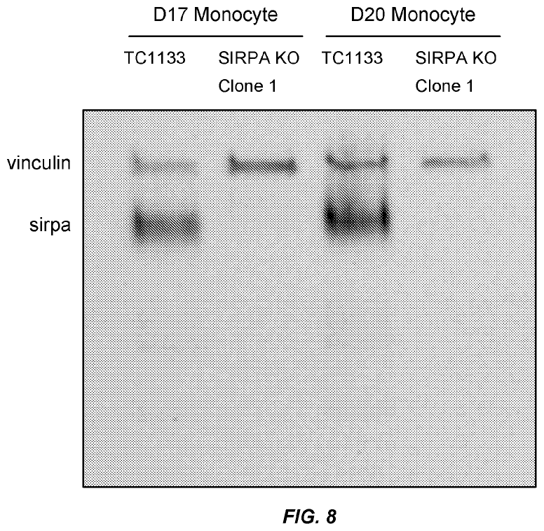

SIRPA KO iPSCs were subsequently differentiated to monocytes. WT (TC1133) and SIRPA KO monocytes were harvested at Day 17 (left) and Day 20 (right) of the differentiation protocol, and lysed for protein. Western blotting confirmed the absence of SIRPA expression in SIRPA KO clone 1 on Day 17 and Day 20 (FIG. 8). Vinculin served as the housekeeping gene, and TC1133 cells served as wild-type control cells that expressed SIRPA.

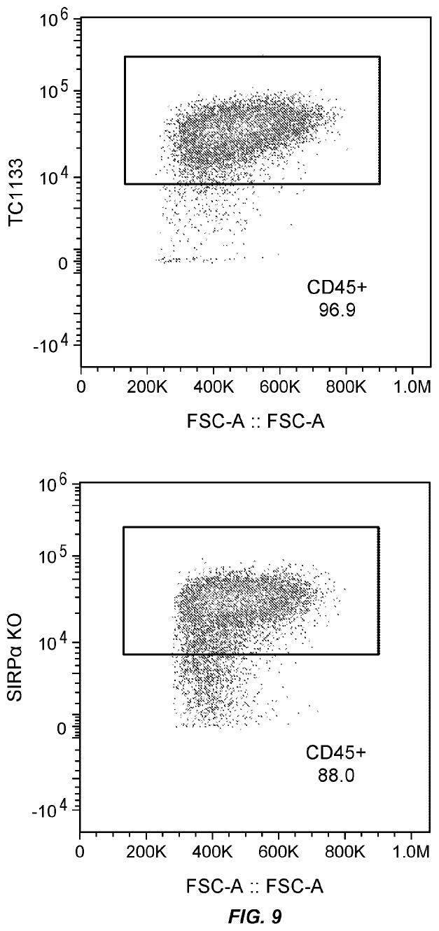

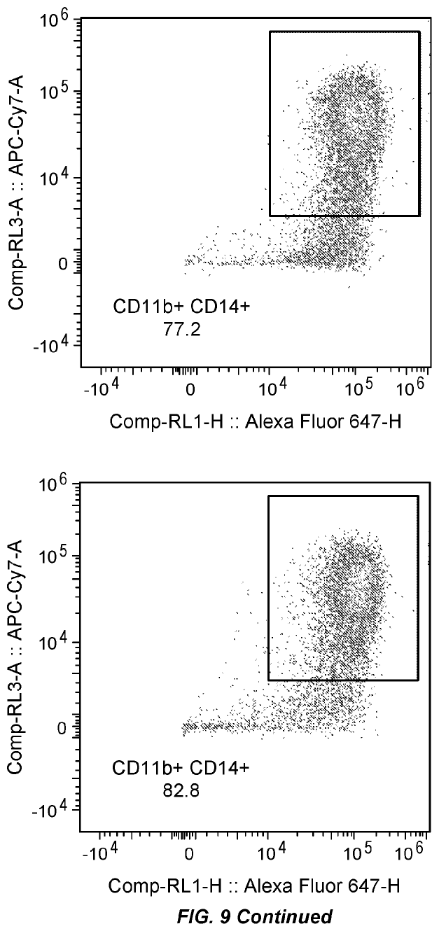

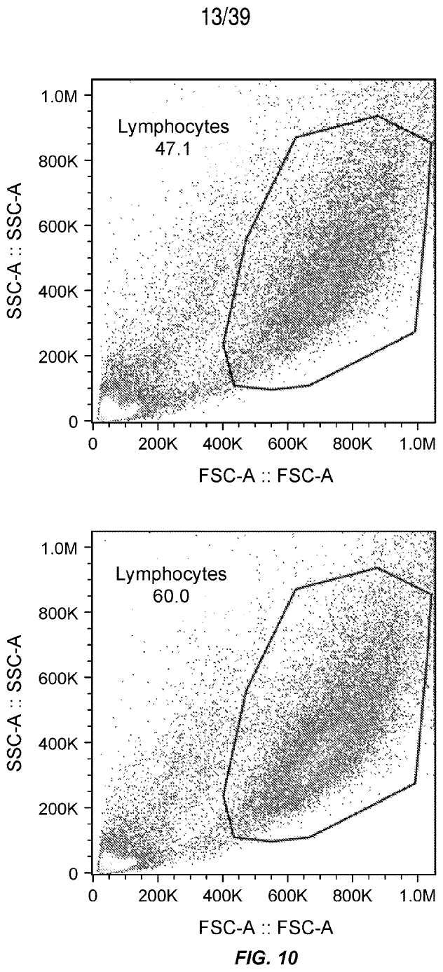

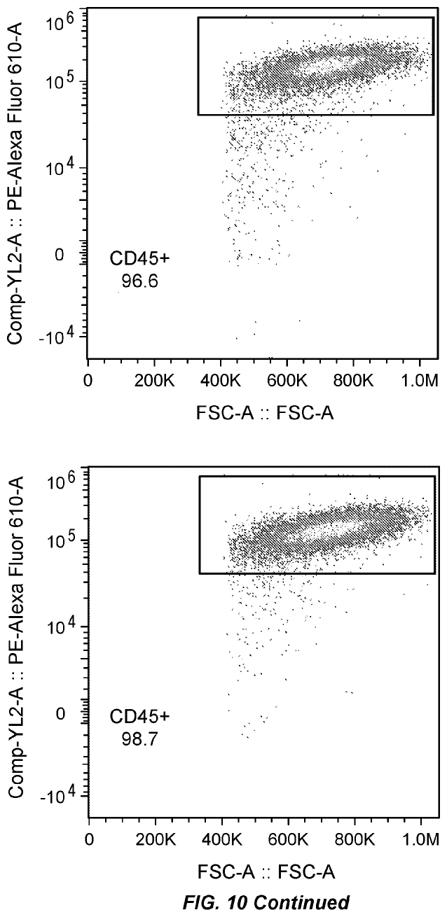

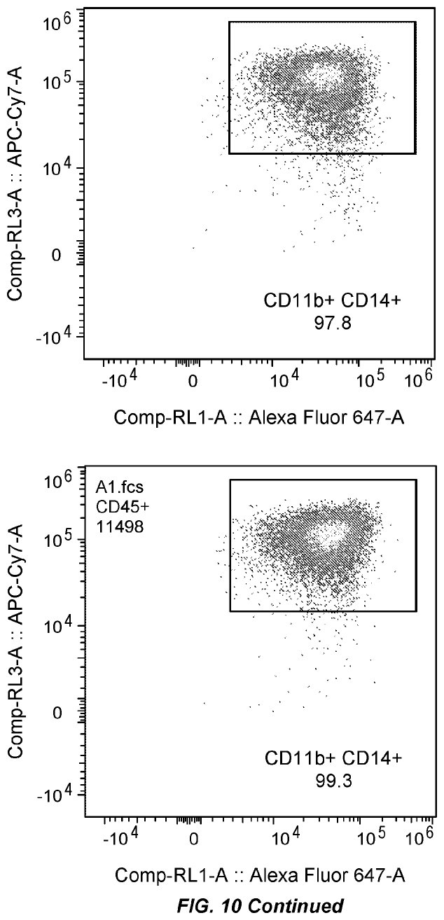

In addition, cells were analyzed by flow cytometry on day Day 17 and Day 20 to confirm monocyte purity. Briefly, live singlets were gated on CD45+, and then CD11b⁺CD14+. CD11b⁺CD14+ confirms monocyte purity. As shown in FIG. 9 and Table 12, SIRPA KO monocytes exhibited similar purity levels as wild-type monocytes. Similar results were observed on Day 20 (FIG. 10 and Table 13).

TABLE 12
Day 17 Monocytes

| | CD45+ (%) | CD11b+ CD14+ |
|---|---|---|
| TC1133 monocytes | 96.9 | 77.2 |
| SIRPA KO monocytes | 88.0 | 82.8 |

TABLE 13
Day 20 Monocytes

| | CD45+ (%) | CD11b+ CD14+ |
|---|---|---|
| TC1133 monocytes | 96.6 | 97.8 |
| SIRPA KO monocytes | 98.7 | 99.3 |

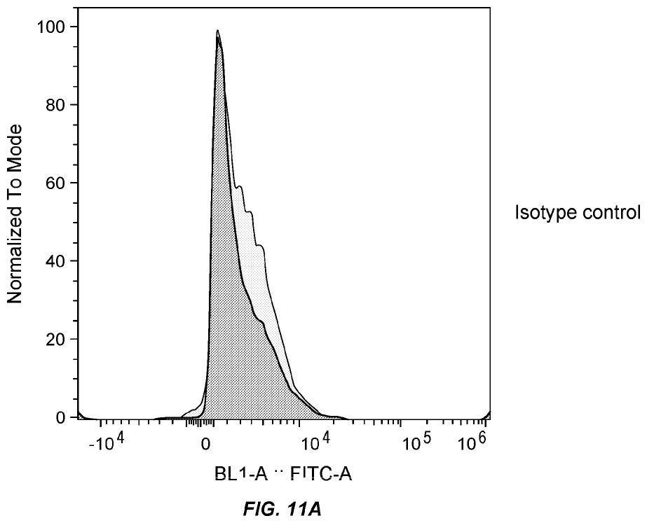

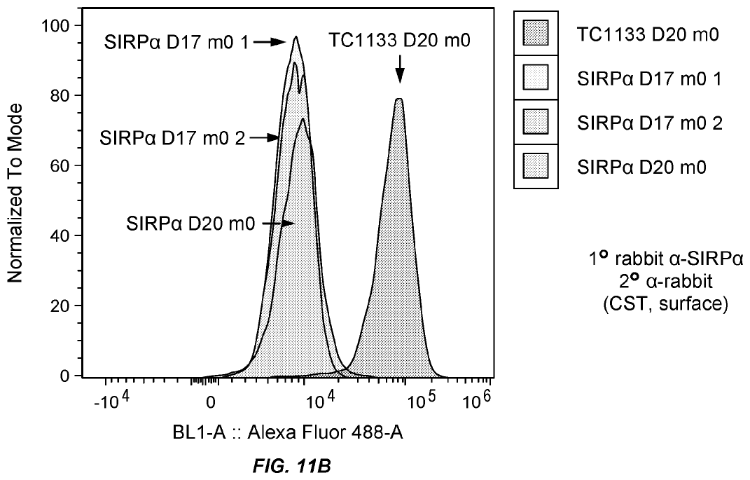

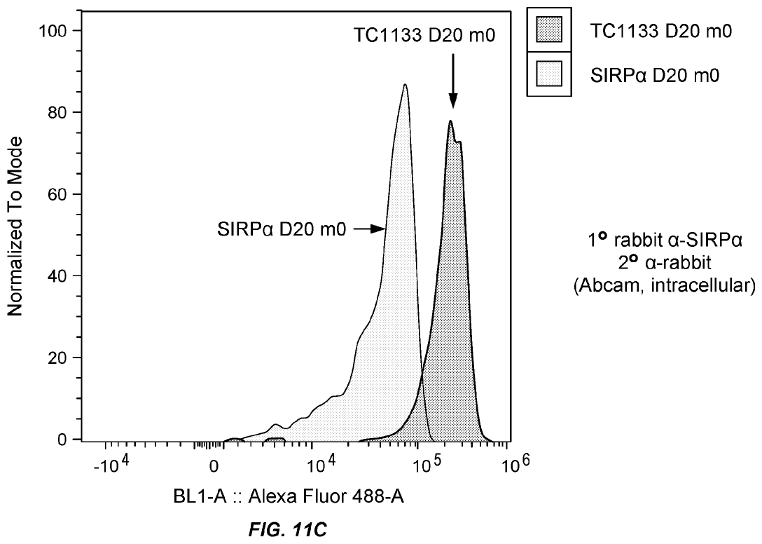

The D17 and D20 iPSC-derived monocytes were further differentiated to iPSC-derived macrophages by treating the monocytes with 100 ng/ml macrophage colony-stimulating factor (M-CSF; also known as colony stimulating factor 1 “CSF1”) for 10 days. Flow cytometry confirmed loss of SIRPα expression in SIRPA KO iPSC-derived macrophages using an antibody that detected cell surface expression of SIRPα (FIG. 11B; Cell Signaling Technologies, CST) or intracellular expression (FIG. 11C; Abcam). Isotype controls were used as a negative control to determine background caused by nonspecific antibody binding (FIG. 11A).

Taken together, this example demonstrates that iPSCs can be genetically modified using CRISPR/MAD7, and successfully differentiated into a monocyte or a macrophage.

### Example 7: SIRPA KO Monocytes have Increased Killing

This example demonstrates that SIRPA KO monocytes have increased killing relative to monocytes expressing SIRPA.

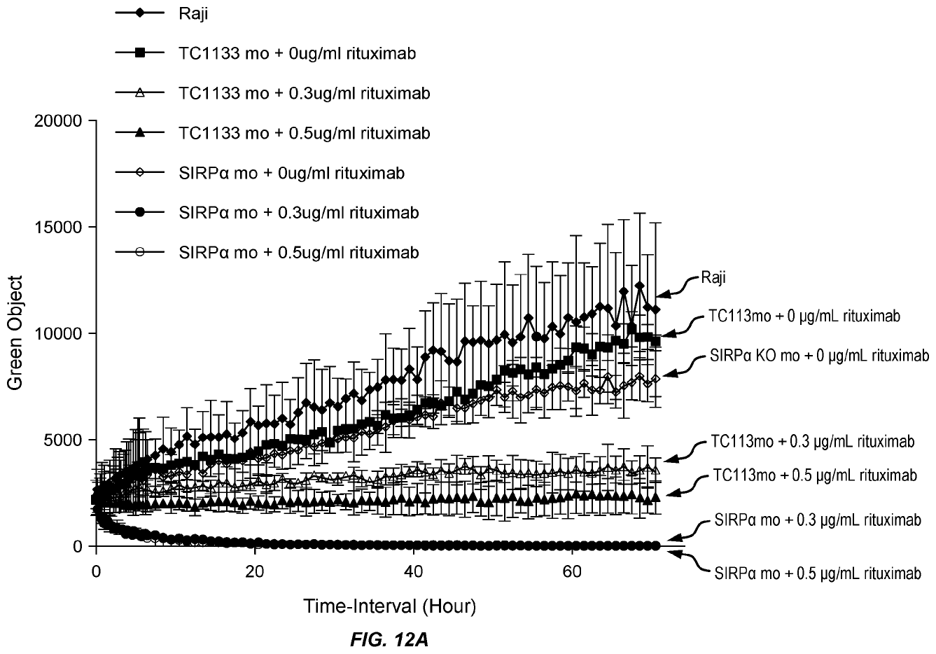

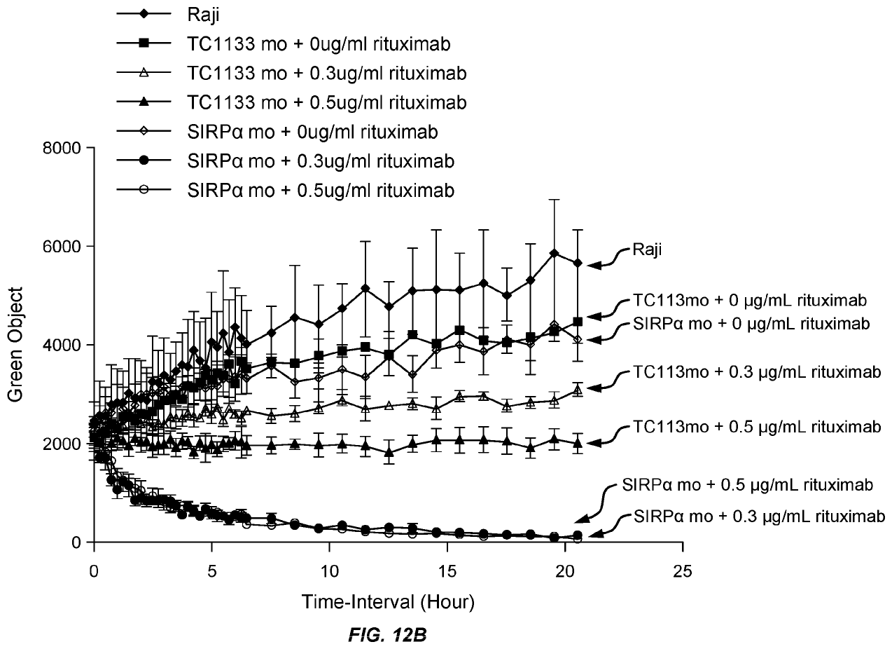

Briefly, SIRPA KO monocyte (“SIRPα mo” or “SIRPα KO mo”) derived from SIRPA KO iPSCs or wild-type control monocytes derived from the TC-113 iPSC-cell line (“TC1133 mo”) were combined with Raji target cells at a 10:1 effector to target ratio. Anti-CD20 (rituximab) antibody was added (0.3 μg/mL or 0.5 μg/mL) and the amount of target cell survival was measured over time (FIG. 12A and FIG. 12B). The results demonstrated that SIRPA KO monocytes have increased killing of Raji cells in comparison to wild-type control monocytes with the addition of 0.3 μg/mL or 0.5 μg/mL rituximab.

In the absence of rituximab, the results demonstrated that SIRPA KO monocytes exhibited a similar amount of target cell killing as the wild-type control monocytes (FIG. 12A and FIG. 12B). Further, both the SIRPA KO monocytes and wild-type TC1133 monocytes exhibited minimal inhibition of the target cell growth in the absence of rituximab, which indicated that the cells exhibited little non-specific killing of the target cells (FIG. 12A and FIG. 12B).

Taken together, this example demonstrates that SIRPA KO monocytes derived from SIRPA KO iPSCs have increased killing relative to monocytes expressing SIRPA derived from wild-type iPSCs.

### Example 8: SIRPA KO Macrophages have Increased Killing

This example demonstrates that SIRPA KO macrophages have increased killing relative to monocytes expressing SIRPA.

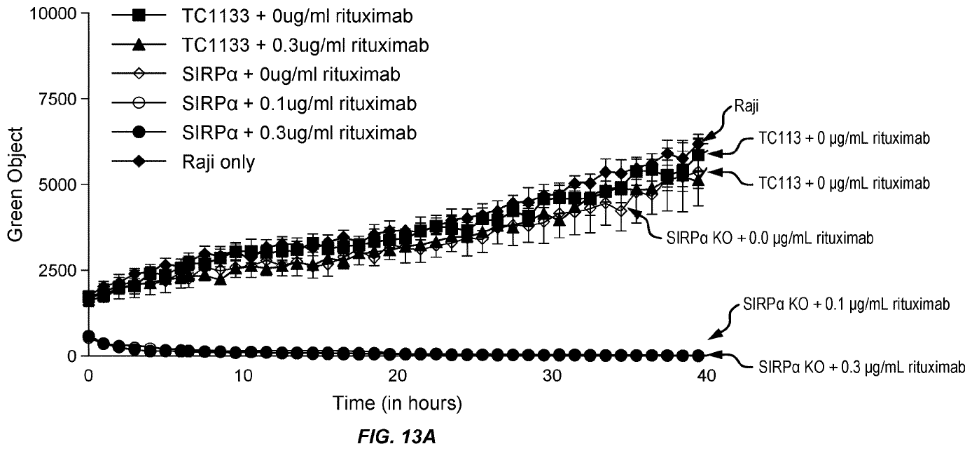

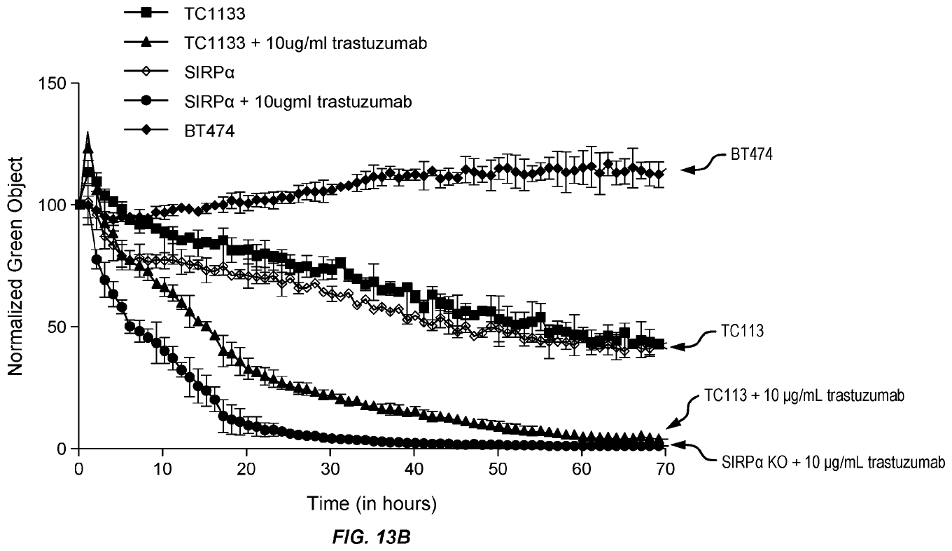

Briefly, SIRPA KO macrophages (“SIRPα” or “SIRPα KO”) derived from SIRPA KO iPSCs or wild-type control macrophages derived from the TC-113 iPSC-cell line (“TC1133”) were combined with Raji target cells at a 3:1 effector to target ratio. Anti-CD20 (rituximab) antibody was added (0.1 μg/mL or 0.3 μg/mL) and the amount of target cell survival was measured over time (FIG. 13A). The results demonstrated that SIRPA KO macrophages derived from SIRPA KO iPSCs have increased killing of Raji cells in comparison to wild-type control macrophages derived from the TC-113 iPSC-cell line upon the addition of 0.1 μg/mL or 0.3 μg/mL rituximab.

In the absence of rituximab, the results demonstrated that SIRPA KO macrophages derived from SIRPA KO iPSCs exhibited a similar amount of target cell killing in the Raji cell model as the wild-type control macrophages (FIG. 13A). Further, both the SIRPA KO macrophages and wild-type TC1133 macrophages exhibited minimal inhibition of the target cell growth in the absence of rituximab in the Raji cell model, which indicated that the cells exhibited little non-specific killing of the target cells (FIG. 13A).

Similar results were obtained in a solid tumor model using BT474 breast cancer cells. Briefly, 100,000 SIRPA KO macrophages (“SIRPα” or “SIRPα KO”) derived from SIRPA KO iPSCs or 100,000 wild-type control macrophages derived from the TC-113 iPSC-cell line (“TC1133”) were combined with 20,000 BT474 target cells at a 5:1 effector cell to target cell ratio. Anti-HER2/neu (trastuzumab) antibody was added (10 μg/mL) and the amount of target cell survival was measured over time (FIG. 13B). The results demonstrated that SIRPA KO macrophages derived from SIRPA KO iPSCs have increased killing of BT474 cells in comparison to wild-type control macrophages derived from the TC-113 iPSC-cell line upon the addition of 10 μg/mL trastuzumab.

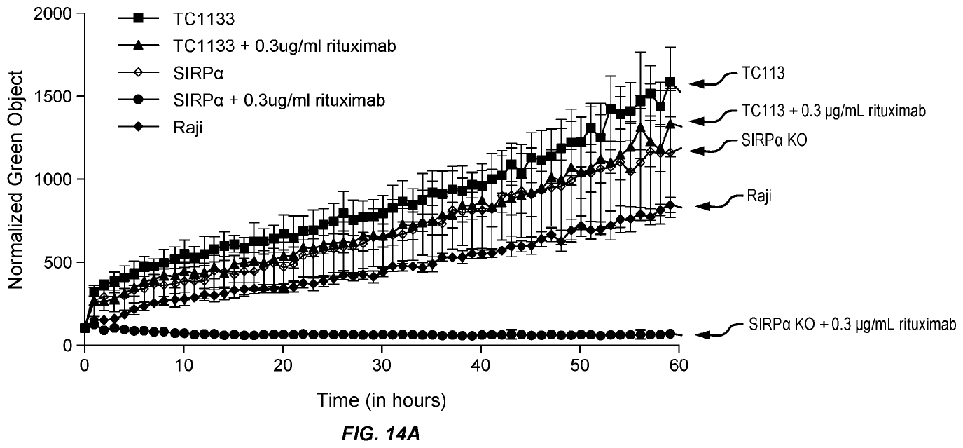

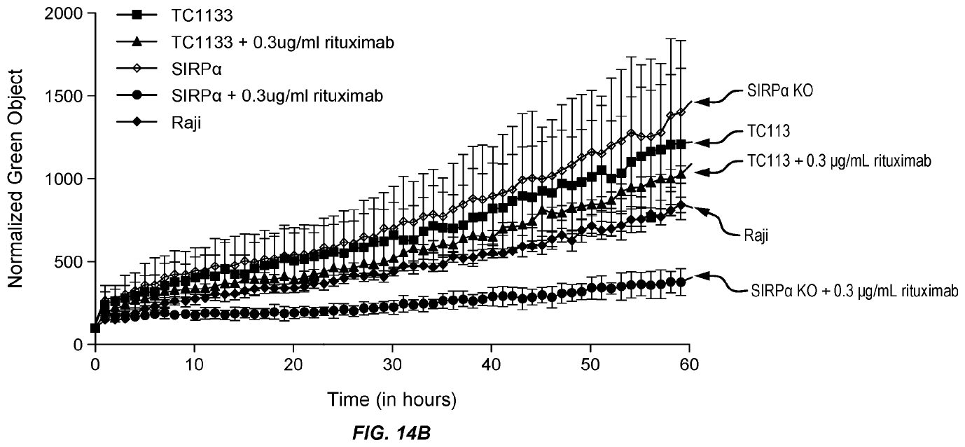

To determine whether SIRPA KO macrophages derived from SIRPA KO iPSCs could exhibit similar efficacy results at lower effector to target ratios, SIRPA KO macrophages derived from SIRPA KO iPSCs or wild-type control macrophages derived from the TC-113 iPSC-cell line (“TC1133”) were combined with Raji target cells at a 3:1 effector to target ratio (FIG. 14A) or a 1:1 effector to target ratio (FIG. 14B). The results demonstrated that that SIRPA KO macrophages derived from SIRPA KO iPSCs have increased killing of Raji cells in comparison to wild-type control macrophages derived from the TC-113 iPSC-cell line upon the addition of 0.3 μg/mL rituximab at both 3:1 and 1:1 effector to target ratios, and showed little non-specific killing of the target cells.

Taken together, this example demonstrates that SIRPA KO macrophages derived from SIRPA KO iPSCs have increased killing relative to macrophages expressing SIRPA derived from wild-type iPSCs. Further, this example demonstrates that SIRPA KO macrophages exhibit efficient target cell killing at lower effector to target ratio.

### Example 9: IMACs Pre-Complexed with Rituximab Kill Raji Cells In Vivo

This example demonstrates that macrophages derived from iPSCs (iMACs) precomplexed with a therapeutic antibody kill target cells in vivo.

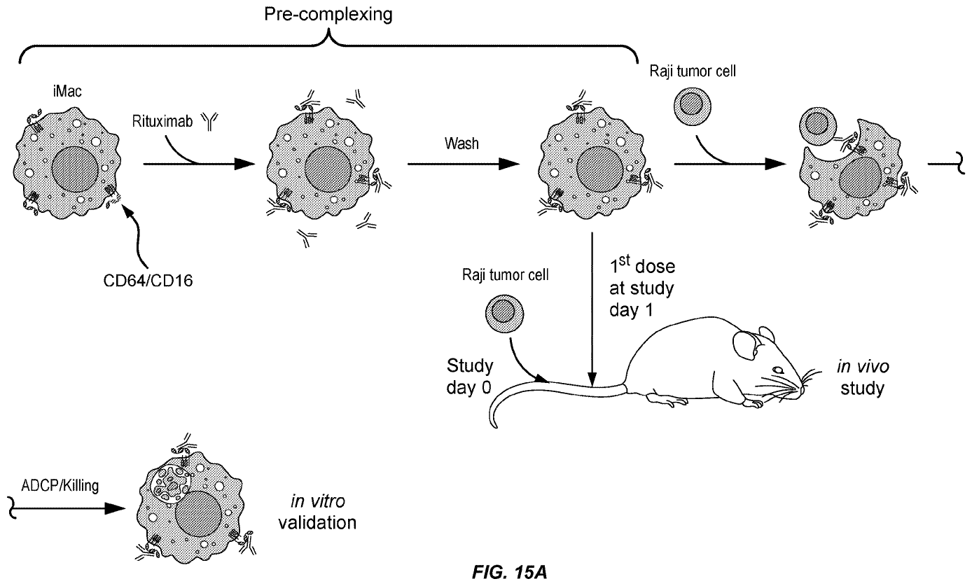

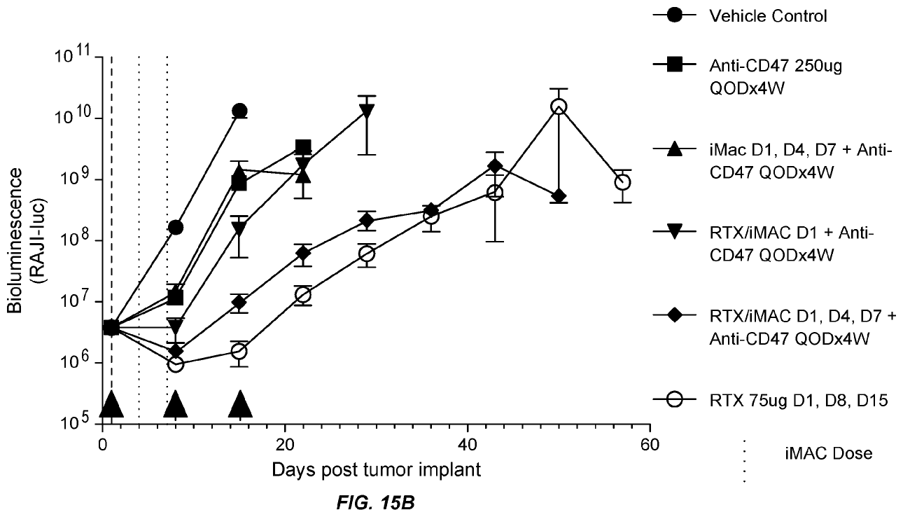

To determine whether macrophages derived from iPSCs are able to synergize with targeting the CD47/SIRPA signaling pathway, six experimental groups of mice were evaluated using the protocols describe in Table 14. Briefly, in one experimental group iMac cells were administered on days 1 and 4, along with treatment of anti-CD47 to disrupt the SIRPA/CD47 signaling pathway (Group 3). Two other experimental groups (Groups 4 and 5) involved iMAC cells precomplexed with rituximab prior to administration, followed by anti-CD47 administration. A schema of pre-complexing is provided in FIG. 15A. Vehicle control (Group 1), anti-CD47 alone (Group 2), and rituximab (RTX) alone (Group 6) were used as controls. The rituximab alone group was administered at a higher dose (75 μg/dose) relative to the predicted dose of rituximab in the precomplexed iMAC cells (~5 μg), and administration occurred for a longer period of time (administered on days 1, 8, and 15) relative to the precomplexed iMAC cells (day 1 only or days 1, 4, and 7).

TABLE 14
In vivo Study Protocols for iMACs

| Treatment Group | Protocol |
|---|---|
| Group 1: Control (n = 5) | Vehicle Control (Days 1, 4, 7) |
| Group 2: Anti-CD47 | Anti-CD47: QOD × 4 weeks; 250 μg/dose (IP) |
| (n = 5) | |
| Group 3: iMAC + | iMAC:1 × 10⁷ cells/mouse administered (IV) on days 1 |
| Anti-CD47 (n = 5) | and 4; 2.3 × 10⁶ cells/mouse administered (IV) on day 7; |
| | Anti-CD47: QOD × 4 weeks; 250 μg/dose (IP) |
| Group 4: RTX/iMAC + | RTX/iMAC: 1 × 10⁷ cells/mouse precomplexed with |
| Anti-CD47 (n = 5) | RTX*, administered together (IV) as single dose on day |
| | 1; |
| | Anti-CD47: QOD × 4 weeks; 250 μg/dose (IP) |
| Group 5: RTX/iMAC + | RTX/iMAC: 1 × 10⁷ cells/mouse precomplexed with |
| Anti-CD47 (n = 5) | RTX*, administered together (IV) on days 1 and 4; |
| | 2.3 × 10⁶ cells/mouse (IV), administered on day 7 |
| | Anti-CD47: QOD × 4 weeks; 250 μg/dose (IP) |
| Group 6: RTX (n = 5) | RTX: 75 μg/dose (IP), administered on days 1, 8, and 15 |

RTX = rituximab;
QOD = every other day;
*predicted RTX injected in precomplexed samples = 5 μg

The results demonstrated that iMACs pre-complexed with rituximab kill Raji cells in vivo. Mice treated with iMACs pre-complexed with rituximab for three days showed improved effector activity (Group 5), relative to Mice treated with iMACs pre-complexed with rituximab for one day (Group 4) (FIG. 15B, diamond and upside-down triangle, respectively). Similarly, both groups of mice treated with iMACs pre-complexed with rituximab exhibited improved effector activity relative to mice treated with anti-CD47 alone (Group 2) or mice treated with iMAC and anti-CD47, but not precomplexed with rituximab (Group 3) (FIG. 15B, square and triangle, respectively). Moreover, the mice treated with iMACs pre-complexed with rituximab for three days showed similar effector activity as mice treated with prolonged high dose rituximab (Group 6), even though the rituximab dose was estimated to be approximately 15 fold lower than the rituximab alone group and treatment occurred for a shorter duration (FIG. 15B, open circle).

Taken together, these results demonstrate that macrophages derived from iPSCs are able to synergize with targeting the CD47/SIRPA signaling pathway to kill Raji cells in vivo.

### Example 10: CAR Knock-In Screening

This example demonstrates that knock-in of CARs in macrophages derived from iPSCs (iMACs) exhibit tumor killing.

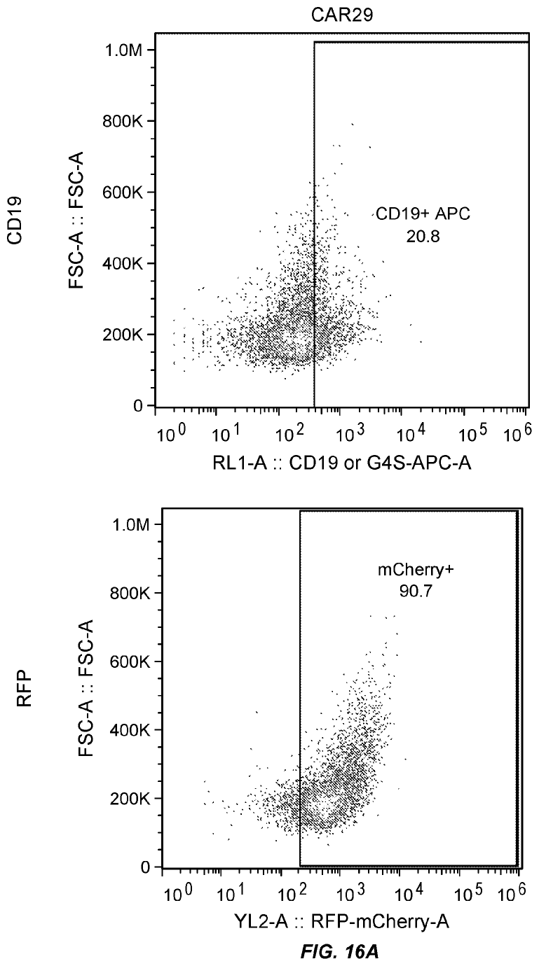

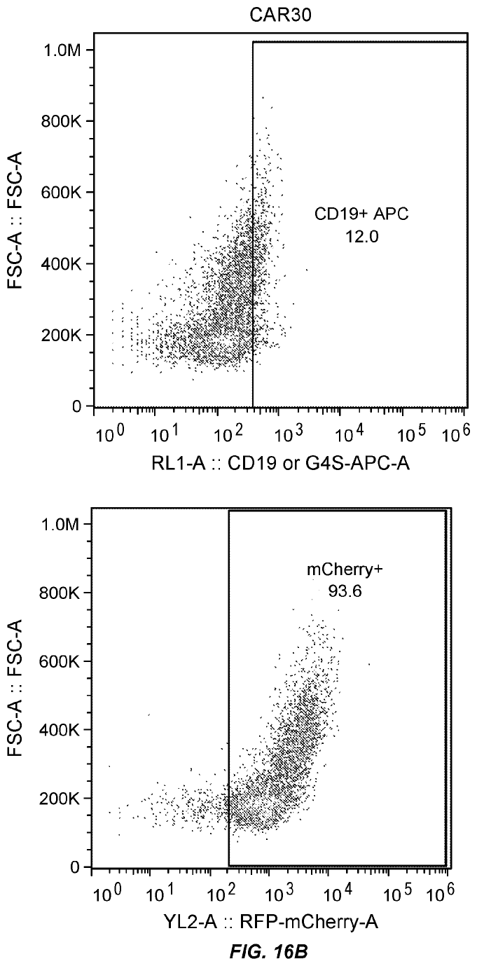

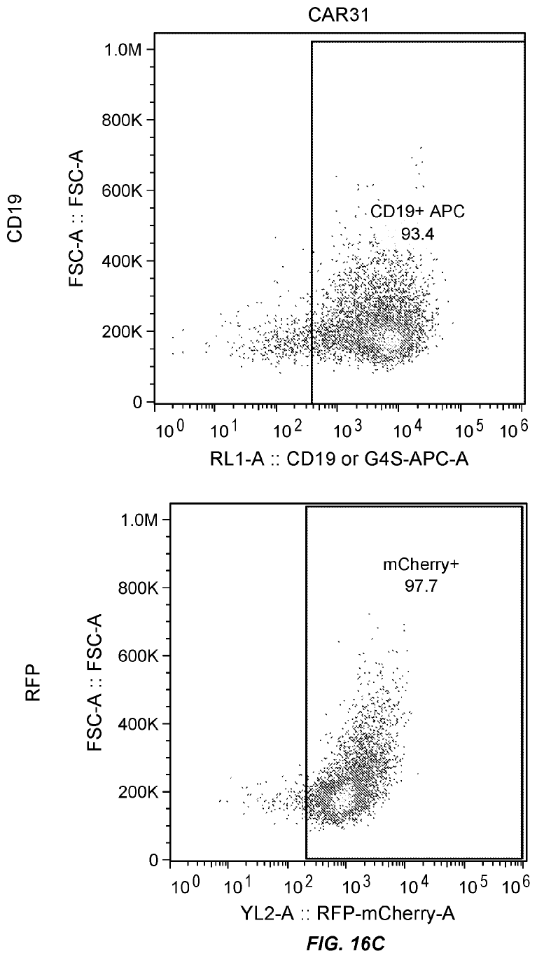

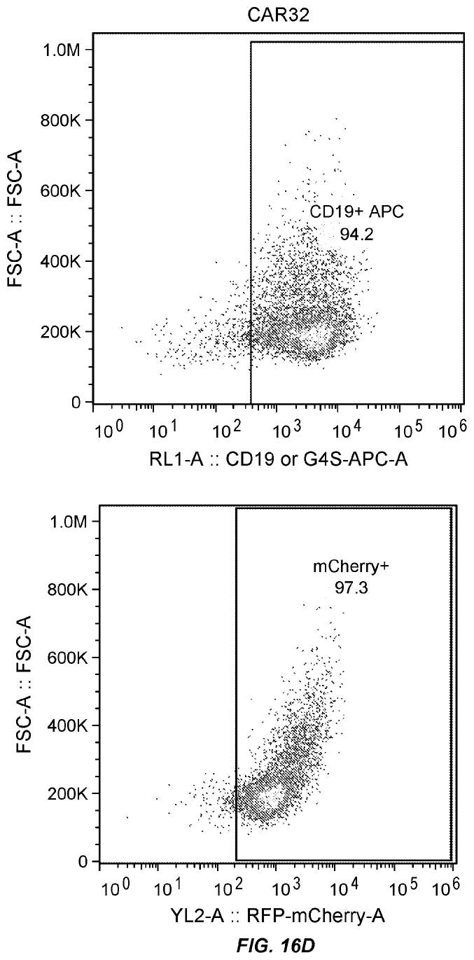

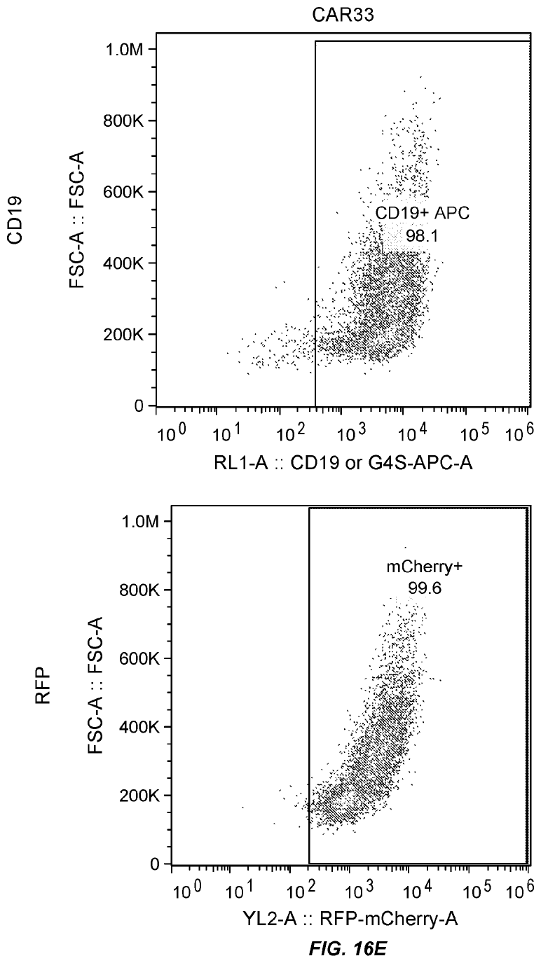

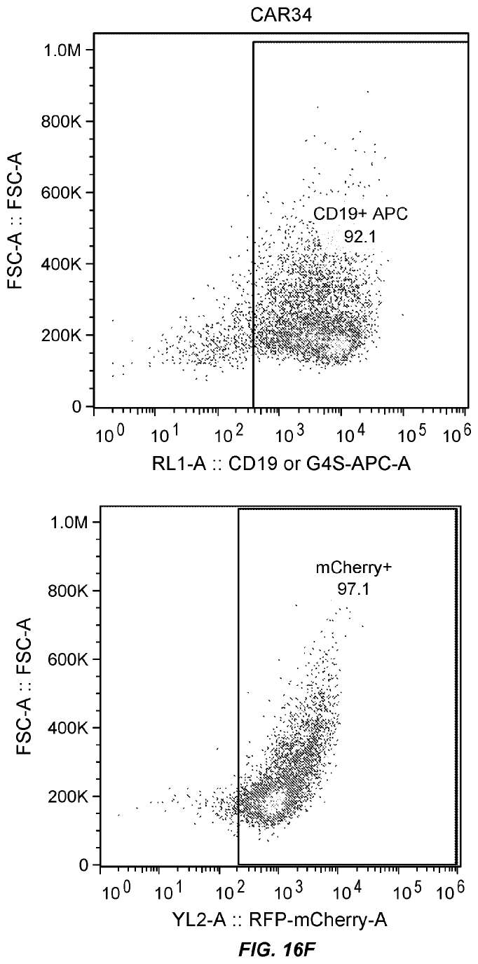

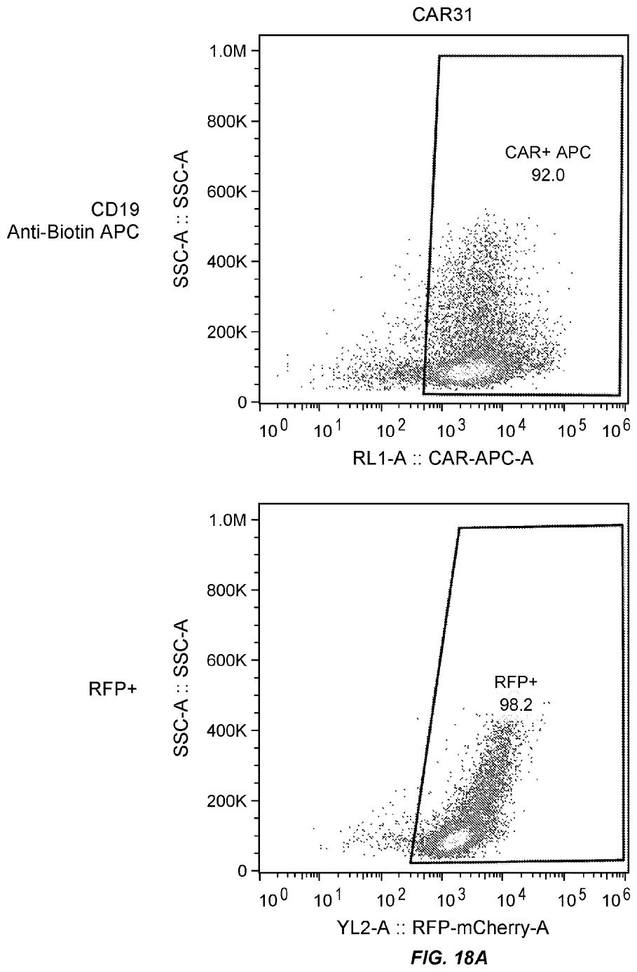

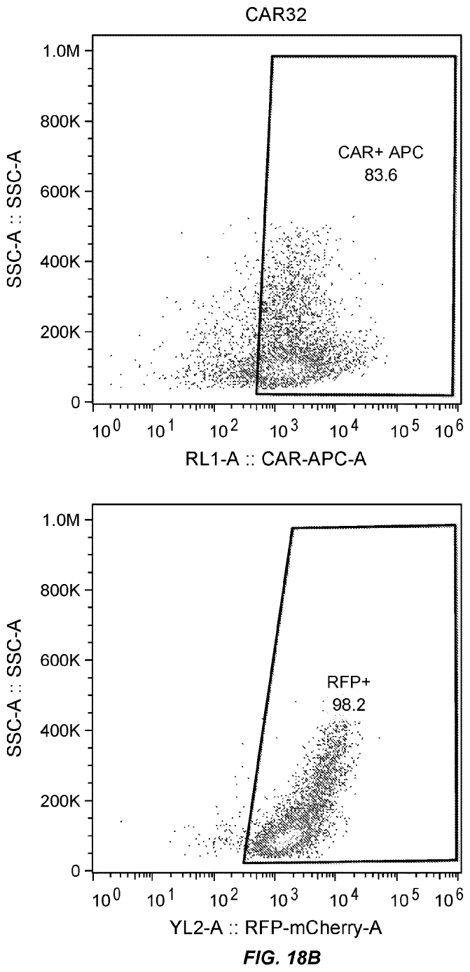

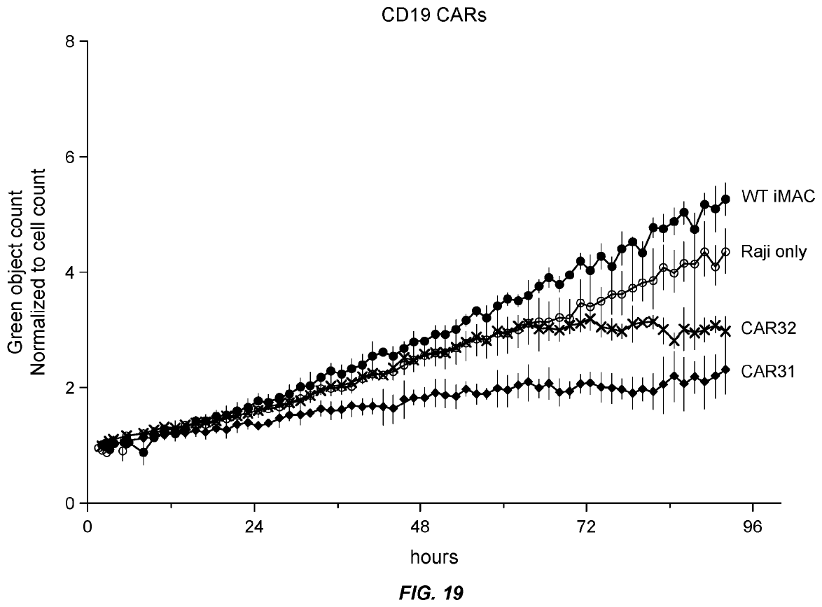

Briefly, anti-CD19 CAR encoding constructs with a red fluorescent protein (RFP) downstream of the CAR were knocked into the AAVS1 safe harbor loci in iPSC. The iPSC pools were enriched by flow sorting CAR positive cells and then differentiated into iMACs. As shown in FIG. 16A-FIG. 16F, knock-in of the CAR resulted in high expression (e.g., >90%) of RFP in the iMAC cells (see also, Table 15). In addition, the anti-CD19 expression was highly expressed in CAR31-CAR34. Similar results were observed in a separate independent experiment for exemplary CAR31 and CAR32 (FIG. 18A-FIG. 18B; see also Table 16)

TABLE 15
CAR expression in iMACs following knock-in

| | CD19 (%) | RFP (%) |
|---|---|---|
| CAR29 | 20.8 | 90.7 |
| CAR30 | 12.0 | 93.6 |
| CAR31 | 93.4 | 97.7 |
| CAR32 | 94.2 | 97.3 |
| CAR33 | 98.1 | 99.6 |
| CAR34 | 92.1 | 97.1 |

TABLE 16
CAR expression in iMACs following knock-in

| | CD19 (%) | RFP (%) |
|---|---|---|
| CAR31 | 92.0 | 98.2 |
| CAR32 | 83.6 | 98.2 |

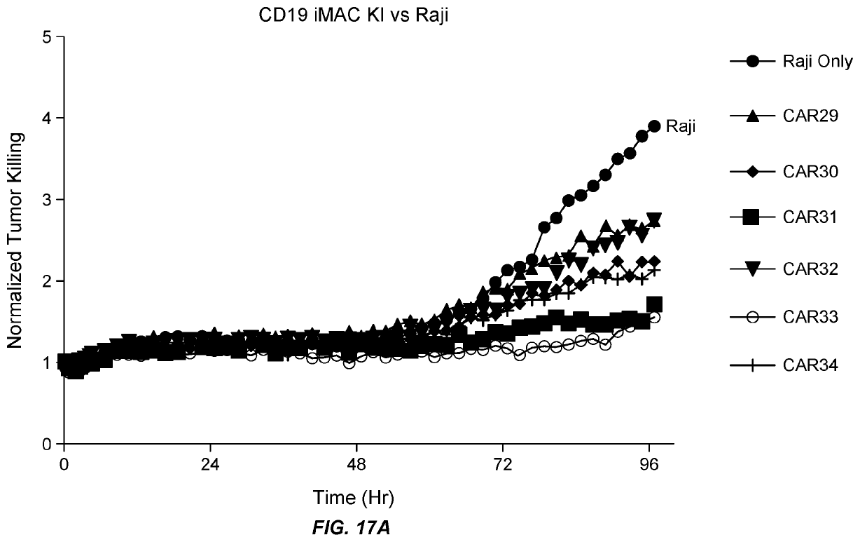

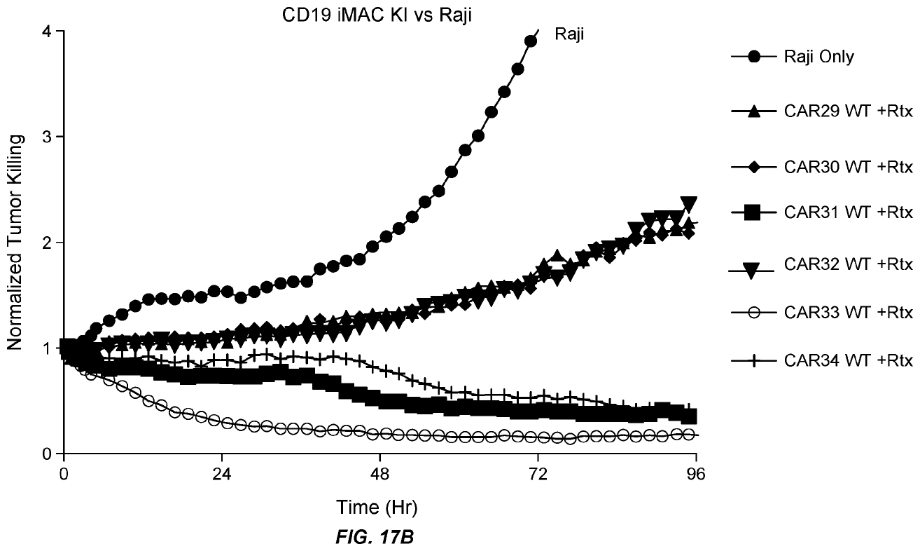

CAR function was tested against CD19+Raji cells over 96 hours, at a. 5:1 ET ratio, with or without 1 ug/mL of Rituximab (anti-CD20 (FIG. 17A and FIG. 17B, respectfully). Of note, CAR31, CAR33, and CAR34, which did not contain CD8 transmembrane domains, functioned better in this assay. Similar results were observed in a separate independent experiment for exemplary CAR31 and CAR32, which slowed down tumor growth, relative to wild-type iMAC cells without a CAR construct (FIG. 18A-FIG. 18B)

Taken together, these results demonstrate that CARs can be successfully knocked into iPSCs and expressed in iMAC cells, and that the iMAC cells expressing CARs described herein are functional.

### Example 11: IMAC CAR in SIRPA KO Macrophages

This example demonstrates that CARs can be expressed in genetically modified iMAC cells using CARs encoded by mRNA.

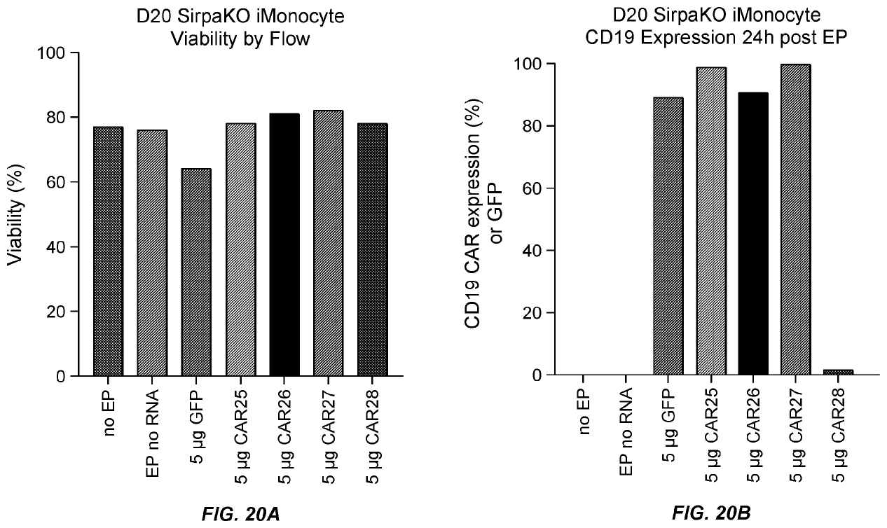

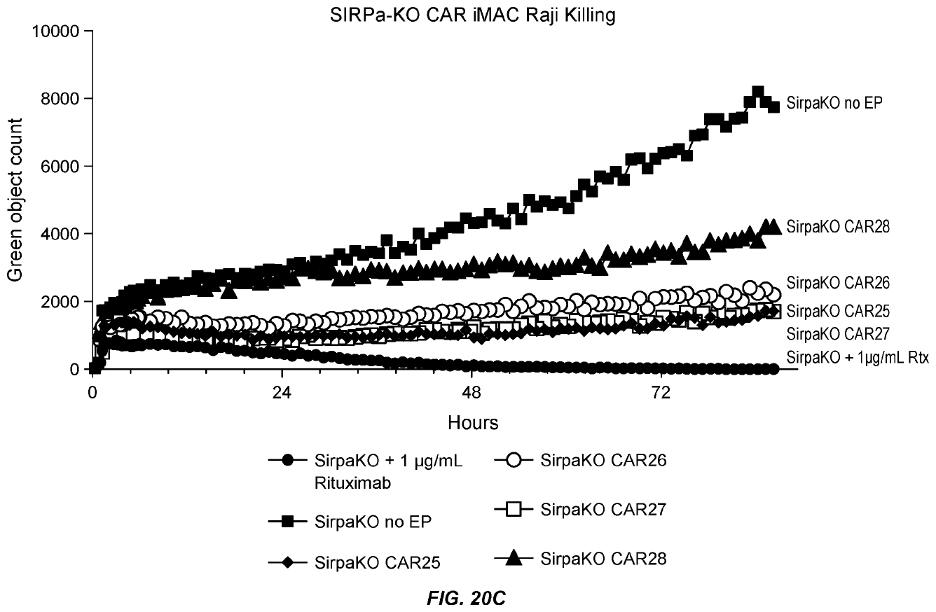

Briefly, anti-CD19 CAR encoding mRNA were electroporated into SIRPA-knockout iMACs using a MAXcyte. 24 h post electroporation cell viability (FIG. 20A) and CAR expression by flow cytometry (FIG. 20B). CAR function was tested against CD19+Raji cells over 96 hours at a. 5:1 E:T ratio (FIG. 20C). The results demonstrated that SIRPA knockout cells expressing CAR (e.g., CAR 25, CAR26, CAR27, or CAR28) exhibited tumor cell killing activity.

These results demonstrate that CARs can be successfully expressed using mRNA, and inhibit the growth of cancer cells.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A polynucleotide encoding a chimeric antigen receptor (CAR), wherein the CAR comprises an antigen recognition moiety, a hinge domain, a transmembrane domain, and an intracellular domain comprising:
(a) a non-lymphoid intracellular signaling domain; and
(b) a CD3ζ intracellular signaling domain, and
wherein the polynucleotide is operatively linked to a promoter.

2. The polynucleotide of claim 1, wherein the promoter is a myeloid-specific promoter.

3. The polynucleotide of claim 2, wherein the myeloid-specific promoter is a native macrophage or a native monocyte promoter.

4. The polynucleotide of claim 2, wherein the myeloid-specific promoter is a synthetic promoter.

5. The polynucleotide of claim 4, wherein the synthetic promoter is selected from the group consisting of synthetic promoter-146 (SP146), synthetic promoter-107 (SP107), and synthetic promoter-60 (SP60).

6. The polynucleotide of claim 1, wherein the promoter is selected from the group consisting of a EF1α, CD36, CD68, SP60, SP107, SP146, CAG, PGK, T7 and CMV promoter.

7. The polynucleotide of any one of claims 1 to 6, wherein the antigen recognition moiety comprises a scFv antigen recognition moiety.

8. The polynucleotide of claim 7, wherein the scFv antigen recognition moiety recognizes a tumor antigen or fragment thereof.

9. The polynucleotide of claim 8, wherein the tumor antigen is selected from the group consisting of HER2/neu, PSMA, Claudin18, CD20, CD5, mesothelin, GD2, and CD19.

10. The polynucleotide of any one of claims 1 to 9, wherein the hinge domain comprises a CD8 hinge domain.

11. The polynucleotide of any one of claims 1 to 10, wherein the transmembrane domain enhances activity of a CAR in a macrophage relative to the CAR having a transmembrane domain consisting of a CD8 transmembrane domain.

12. The polynucleotide of claim 11, wherein the increase in activity comprises an increase in secretion of a pro-inflammatory cytokine.

13. The polynucleotide of claim 12, wherein the increase in secretion of the pro-inflammatory cytokine is about 10-fold to about 100-fold.

14. The polynucleotide of claim 13, wherein the increase in secretion of the pro-inflammatory cytokine is about 25-fold.

15. The polynucleotide of claim 13, wherein the increase in secretion of the pro-inflammatory cytokine is about 50-fold.

16. The polynucleotide of claim 13, wherein the increase in secretion of the pro-inflammatory cytokine is about 75-fold.

17. The polynucleotide of claim 13, wherein the increase in secretion of the pro-inflammatory cytokine is about 100-fold.

18. The polynucleotide of claim 13, wherein the increase in secretion of the pro-inflammatory cytokine is more than about 100-fold.

19. The polynucleotide of claim 12, wherein the pro-inflammatory cytokine is selected from the group consisting of CXCL10, CCL1, CCL2, CCL22, MIP-1α/CCL3, IL-10, IL-1β, IFNγ, IL-6, TNFA, and CCL20, or a combination thereof.

20. The polynucleotide of claim 12, wherein the pro-inflammatory cytokine comprises MIP-1α/CCL3.

21. The polynucleotide of claim 19, wherein the increase in secretion of the pro-inflammatory cytokine comprises a composite score of two of more cytokines.

22. The polynucleotide of any one of claims 12 to 21, wherein the secretion of the pro-inflammatory cytokine is measured by ELISA after culturing the macrophage for 24 hours in the presence of a target antigen.

23. The polynucleotide of any one of claims 11 to 22, wherein the increase in activity comprises an increase in macrophage phagocytosis.

24. The polynucleotide of claim 23, wherein the increase in macrophage phagocytosis is about 10% to about 50%.

25. The polynucleotide of claim 24, wherein the increase is about 10%.

26. The polynucleotide of claim 24, wherein the increase is about 20%.

27. The polynucleotide of claim 24, wherein the increase is about 30%.

28. The polynucleotide of claim 24, wherein the increase is about 40%.

29. The polynucleotide of claim 24, wherein the increase is more than about 40%.

30. The polynucleotide of any one of claims 23 to 29, wherein phagocytosis is measured by flow cytometry after about four hours of co-incubation of a macrophage expressing the CAR and a target cell expressing a target antigen.

31. The polynucleotide of any one of claims 1 to 30, wherein the transmembrane domain is selected from the group consisting of IL-1R1, VEGFR2, IFNγR1, CSF3R, CSF2R, SLAMF7, DECTIN-1, DECTIN-3, TLR4, CSF1R, CD8, CD28, MINCLE, MDL1, BDCA2, DAP12, CD11b, CD86, and CD40.

32. The polynucleotide of any one of claims 1 to 31, wherein the intracellular domain increases activity of a CAR in a macrophage relative to the CAR having an intracellular domain consisting of a CD3ζ intracellular signaling domain.

33. The polynucleotide of claim 32, wherein the increase in activity comprises an increase in secretion of a pro-inflammatory cytokine.

34. The polynucleotide of claim 33, wherein the increase in secretion of the pro-inflammatory cytokine is about 10-fold to about 100-fold.

35. The polynucleotide of claim 33, wherein the increase in secretion of the pro-inflammatory cytokine is about 25-fold.

36. The polynucleotide of claim 33, wherein the increase in secretion of the pro-inflammatory cytokine is about 50-fold.

37. The polynucleotide of claim 33, wherein the increase in secretion of the pro-inflammatory cytokine is about 75-fold.

38. The polynucleotide of claim 33, wherein the increase in secretion of the pro-inflammatory cytokine is about 100-fold.

39. The polynucleotide of claim 33, wherein the increase in secretion of the pro-inflammatory cytokine is more than about 100-fold.

40. The polynucleotide of claim 33, wherein the pro-inflammatory cytokine is selected from the group consisting of CXCL10, CCL1, CCL2, CCL22, MIP-1α/CCL3, IL-10, IL-1β, IFNγ, IL-6, TNFA, and CCL20, or a combination thereof.

41. The polynucleotide of claim 33, wherein the pro-inflammatory cytokine comprises MIP-1α/CCL3.

42. The polynucleotide of claim 40, wherein the increase in secretion of the pro-inflammatory cytokine comprises a composite score of two of more pro-inflammatory cytokines.

43. The polynucleotide of any one of claims 33 to 42, wherein the secretion of the pro-inflammatory cytokine is measured by ELISA after culturing the macrophage for 24 hours in the presence of a target antigen.

44. The polynucleotide of any one of claims 32 to 43, wherein the increase in activity comprises an increase in macrophage phagocytosis.

45. The polynucleotide of claim 44, wherein the increase in macrophage phagocytosis is about 10% to about 50%.

46. The polynucleotide of claim 45, wherein the increase is about 10%.

47. The polynucleotide of claim 45, wherein the increase is about 20%.

48. The polynucleotide of claim 45, wherein the increase is about 30%.

49. The polynucleotide of claim 45, wherein the increase is about 40%.

50. The polynucleotide of claim 45, wherein the increase is more than about 40%.

51. The polynucleotide of any one of claims 44 to 50, wherein phagocytosis is measured by flow cytometry after about four hours of co-incubation of a macrophage expressing the CAR and a target cell expressing a target antigen.

52. The polynucleotide of any one of claims 1 to 51, wherein the one or more non-lymphoid intracellular signaling domains are selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, and CD80/B7-1.

53. A polynucleotide encoding a chimeric antigen receptor (CAR), wherein the CAR comprises
(a) an antigen recognition moiety;
(b) a CD8 hinge domain,
(c) a transmembrane domain, wherein the transmembrane domain is selected from the group consisting of
(i) a CD8 transmembrane domain;
(ii) a SLAMF7 transmembrane domain;
(iii) a CD86 transmembrane domain;
(iv) a DECTIN-1 transmembrane domain; and
(v) a CSF1R transmembrane domain;
(d) a first intracellular signaling domain, wherein the first intracellular signaling domain is selected from the group consisting of (i) a CD86 intracellular signaling domain;
(ii) a Lox1c intracellular signaling domain;
(iii) a 2B4 intracellular signaling domain;
(iv) a DECTIN-1 intracellular signaling domain; and
(v) a CD40 intracellular signaling domain; and
(e) a second intracellular domain comprising a CD3ζ intracellular signaling domain,
wherein the polynucleotide is operatively linked to a promoter.

54. The polynucleotide of claim 53, wherein the polynucleotide encodes for a hinge domain comprising an amino acid sequence of SEQ ID NO:1.

55. The polynucleotide of claim 54, wherein the transmembrane domain comprises a CD8 transmembrane domain.

56. The polynucleotide of claim 55, wherein the first intracellular signaling domain comprises a CD86 intracellular signaling domain.

57. The polynucleotide of claim 55, wherein the first intracellular signaling domain comprises a Lox1c intracellular signaling domain.

58. The polynucleotide of claim 54, wherein the transmembrane domain comprises a SLAMF7 transmembrane domain.

59. The polynucleotide of claim 58, wherein the first intracellular signaling domain comprises a CD86 intracellular signaling domain.

60. The polynucleotide of claim 54, wherein the transmembrane domain comprises a CD86 transmembrane domain.

61. The polynucleotide of claim 60, the first intracellular signaling domain comprises a 2B4 intracellular signaling domain.

62. The polynucleotide of claim 54, wherein the transmembrane domain comprises a DECTIN-1 transmembrane domain.

63. The polynucleotide of claim 54, wherein the transmembrane domain comprises a CSF1R transmembrane domain.

64. The polynucleotide of claim 62, wherein the first intracellular signaling domain comprises a DECTIN-1 intracellular signaling domain.

65. The polynucleotide of claim 55, wherein the first intracellular signaling domain comprises a CD40 intracellular signaling domain.

66. The polynucleotide of claim 53, wherein the polynucleotide encodes for a hinge domain comprising an amino acid sequence of SEQ ID NO:4.

67. The polynucleotide of claim 66, wherein the first intracellular signaling domain comprises a CD86 intracellular signaling domain.

68. The polynucleotide of claim 67, wherein the transmembrane domain comprises a SLAMF7 transmembrane domain.

69. The polynucleotide of any one of claims 53 to 68, wherein the promoter is a constitutive promoter.

70. The polynucleotide of claim 69, wherein the constitutive promoter is a EF1α promoter, a CAG promoter, or a T7 promoter.

71. The polynucleotide of any one of claims 53 to 68, wherein the promoter is a myeloid-specific promoter.

72. The polynucleotide of claim 71, wherein the myeloid-specific promoter is a native macrophage promoter or a native monocyte promoter.

73. The polynucleotide of claim 71, wherein the myeloid-specific promoter is a synthetic promoter.

74. The polynucleotide of claim 73, wherein the synthetic promoter is selected from the group consisting of synthetic promoter-146 (SP146), synthetic promoter-107 (SP107), and synthetic promoter-60 (SP60).

75. The polynucleotide of claim 74, wherein the synthetic promoter is SP146.

76. The polynucleotide of any one of claims 53 to 75, wherein the antigen recognition moiety comprises a scFv antigen recognition moiety.

77. The polynucleotide of claim 76, wherein the scFv antigen recognition moiety recognizes a tumor antigen or fragment thereof.

78. The polynucleotide of claim 77, wherein the tumor antigen is selected from your group consisting of HER2/neu, PSMA, Claudin18, CD20, CD5, mesothelin, GD2, and CD19.

79. A vector comprising the polynucleotide of any one of claims 1 to 78.

80. The vector of claim 79, wherein the vector comprises DNA or RNA.

81. The vector of claim 79 or 80, wherein the vector is a plasmid vector.

82. The vector of claim 79 or 80, wherein the vector is a viral vector.

83. The vector of claim 82, wherein the viral vector is selected from the group consisting of an adenoviral vector, a lentiviral vector, and a retroviral vector.

84. The vector of claim 82, wherein the viral vector is a lentiviral vector.

85. A polypeptide comprising:
(a) an antigen recognition moiety polypeptide;
(b) a CD8 hinge domain polypeptide;
(c) a transmembrane domain polypeptide, wherein the transmembrane domain is selected from the group consisting of
(i) a CD8 transmembrane domain;
(ii) a SLAMF7 transmembrane domain;
(iii) a CD86 transmembrane domain;
(iv) a DECTIN-1 transmembrane domain; and
(v) a CSF1R transmembrane domain;
(d) a first intracellular signaling domain polypeptide, wherein the first intracellular signaling domain is selected from the group consisting of:
(i) a CD86 intracellular signaling domain;
(ii) a Lox1c intracellular signaling domain,
(iii) a 2B4 intracellular signaling domain;
(iv) a DECTIN-1 intracellular signaling domain; and
(v) a CD40 intracellular signaling domain polypeptide; and
(e) a second intracellular domain polypeptide comprising a CD3ζ intracellular signaling domain.

86. The polypeptide of claim 85, wherein the hinge domain has an amino acid sequence of SEQ ID NO:1.

87. The polypeptide of claim 86, wherein the transmembrane domain polypeptide comprises a CD8 transmembrane domain polypeptide.

88. The polypeptide of claim 87, wherein the CD8 transmembrane domain polypeptide has an amino acid sequence of SEQ ID NO:11.

89. The polypeptide of claim 87 or 88, wherein the first intracellular signaling domain polypeptide comprises a CD86 intracellular signaling domain polypeptide.

90. The polypeptide of claim 89, wherein the CD86 intracellular signaling domain polypeptide has an amino acid sequence of SEQ ID NO:40.

91. The polypeptide of claim 87 or 88, wherein the first intracellular signaling domain polypeptide comprises a Lox1c intracellular signaling domain polypeptide.

92. The polypeptide of claim 91, wherein the Lox1c intracellular signaling domain polypeptide has an amino acid sequence of SEQ ID NO:41.

93. The polypeptide of claim 86, wherein the transmembrane domain polypeptide comprises a SLAMF7 transmembrane domain polypeptide.

94. The polypeptide of claim 93, wherein the SLAMF7 transmembrane domain polypeptide has an amino acid sequence of SEQ ID NO:12.

95. The polypeptide of claim 93 or 94, wherein the first intracellular signaling domain polypeptide comprises a CD86 intracellular signaling domain polypeptide.

96. The polypeptide of claim 95, wherein the CD86 intracellular signaling domain polypeptide has an amino acid sequence of SEQ ID NO:40.

97. The polypeptide of claim 86, wherein the transmembrane domain polypeptide comprises a CD86 transmembrane domain polypeptide.

98. The polypeptide of claim 97, wherein the CD86 transmembrane domain polypeptide has an amino acid sequence of SEQ ID NO:13.

99. The polypeptide of claim 97 or 98, wherein the first intracellular signaling domain polypeptide comprises a 2B4 intracellular signaling domain polypeptide.

100. The polypeptide of claim 99, wherein the 2B4 intracellular signaling domain polypeptide has an amino acid sequence of SEQ ID NO:42.

101. The polypeptide of claim 86, wherein the transmembrane domain polypeptide comprises a DECTIN-1 transmembrane domain polypeptide.

102. The polypeptide of claim 101, wherein the DECTIN-1 transmembrane domain polypeptide has an amino acid sequence of SEQ ID NO:14.

103. The polypeptide of claim 101 or 102, wherein the first intracellular signaling domain polypeptide comprises a DECTIN-1 intracellular signaling domain polypeptide.

104. The polypeptide of claim 103, wherein the DECTIN-1 intracellular signaling domain polypeptide has an amino acid sequence of SEQ ID NO:43.

105. The polypeptide of claim 85, wherein the hinge domain polypeptide comprises SEQ ID NO: 4.

106. The polypeptide of claim 105, wherein the transmembrane domain polypeptide comprises a SLAMF7 transmembrane domain.

107. The polypeptide of claim 106, the SLAMF7 transmembrane domain polypeptide has an amino acid sequence of SEQ ID NO:12.

108. The polypeptide of claim 106 or 107, wherein the first intracellular signaling domain polypeptide comprises a CD86 intracellular signaling domain polypeptide.

109. The polypeptide of claim 108, wherein the CD86 intracellular signaling domain polypeptide has an amino acid sequence of SEQ ID NO:40.

110. The polypeptide of any one of claims to 85 to 109, wherein the antigen recognition moiety polypeptide comprises a scFv antigen recognition moiety polypeptide.

111. The polypeptide of claim 110, wherein the scFv antigen recognition moiety polypeptide recognizes a tumor antigen or fragment thereof.

112. The polynucleotide of claim 111, wherein the tumor antigen is selected from your group consisting of HER2/neu, PSMA, Claudin18, CD20, CD5, mesothelin, GD2, and CD19.

113. A polypeptide having an amino acid sequence comprising or consisting of SEQ ID NO: 97.

114. A polypeptide having an amino acid sequence comprising or consisting of SEQ ID NO: 98.

115. A polypeptide having an amino acid sequence comprising or consisting of SEQ ID NO: 99.

116. A polypeptide having an amino acid sequence comprising or consisting of SEQ ID NO: 100.

117. A polypeptide having an amino acid sequence comprising or consisting of SEQ ID NO: 101.

118. A polypeptide having an amino acid sequence comprising or consisting of SEQ ID NO: 102.

119. A polypeptide having an amino acid sequence comprising or consisting of SEQ ID NO: 103.

120. A polypeptide having an amino acid sequence comprising or consisting of SEQ ID NO: 104.

121. A polypeptide having an amino acid sequence comprising or consisting of SEQ ID NO: 105.

122. A polypeptide having an amino acid sequence comprising or consisting of SEQ ID NO: 106.

123. A polypeptide having an amino acid sequence comprising or consisting of SEQ ID NO: 107.

124. A polypeptide having an amino acid sequence comprising or consisting of SEQ ID NO: 108.

125. A polypeptide having an amino acid sequence comprising or consisting of SEQ ID NO: 110.

126. A polypeptide having an amino acid sequence comprising or consisting of SEQ ID NO: 111.

127. A polypeptide having an amino acid sequence comprising or consisting of SEQ ID NO: 112.

128. A polypeptide having an amino acid sequence comprising or consisting of SEQ ID NO: 113.

129. A polypeptide having an amino acid sequence comprising or consisting of SEQ ID NO: 114.

130. A polypeptide having an amino acid sequence comprising or consisting of SEQ ID NO: 115.

131. A polypeptide having an amino acid sequence comprising or consisting of SEQ ID NO: 116.

132. A polypeptide having an amino acid sequence comprising or consisting of SEQ ID NO: 117.

133. A polypeptide having an amino acid sequence comprising or consisting of SEQ ID NO: 118.

134. A polypeptide having an amino acid sequence comprising or consisting of SEQ ID NO: 119.

135. A polypeptide having an amino acid sequence comprising or consisting of SEQ ID NO: 120.

136. A polypeptide having an amino acid sequence comprising or consisting of SEQ ID NO: 121.

137. A polypeptide having an amino acid sequence comprising or consisting of SEQ ID NO: 125.

138. A polypeptide having an amino acid sequence comprising or consisting of SEQ ID NO: 126.

139. A polypeptide having an amino acid sequence comprising or consisting of SEQ ID NO: 127.

140. A polypeptide having an amino acid sequence comprising or consisting of SEQ ID NO: 128.

141. A polypeptide having an amino acid sequence comprising or consisting of SEQ ID NO: 129.

142. A polypeptide having an amino acid sequence comprising or consisting of SEQ ID NO: 130.

143. A polypeptide having an amino acid sequence comprising or consisting of SEQ ID NO: 131.

144. A polypeptide having an amino acid sequence comprising or consisting of SEQ ID NO: 132.

145. A modified cell, wherein the modified cell comprises the polynucleotide of any one of claims 1 to 78, the vector of any one of claims 79 to 84, or the polypeptide of any one of claims 85 to 144, and
wherein the modified cell is a monocyte, a macrophage, or an induced pluripotent stem cell (iPSC).

146. The modified cell of claim 145, wherein the modified cell is an iPSC.

147. The modified cell of claim 145, wherein the modified cell is a macrophage.

148. The modified cell of claim 145, wherein the modified cell is a monocyte.

149. The modified cell of claim 147 or 148, wherein the modified cell is derived from an iPSC.

150. The modified cell of any one of claims 145 to 149, wherein the modified cell is a human cell.

151. The modified cell of any one of claims 145 to 150, comprising a genetic disruption of:
(a) a signal regulatory protein alpha (SIRPA) gene;
(b) a cytokine inducible SH2 containing protein (CISH) gene; and/or
(c) a sialic acid-binding Ig-like lectin 10 (SIGLEC10) gene.

152. The modified cell of claim 151, wherein the modified cell comprises genetic disruption of SIRPA.

153. The modified cell of claim 151 or 152, wherein the modified cell comprises genetic disruption of CISH.

154. The modified cell of any one of claims 151 to 153, wherein the modified cell comprises genetic disruption of SIGLEC10.

155. The modified cell of any one of claims 147 to 154, wherein the modified cell comprises an agent capable of preventing or reducing interaction between CD47 and SIRPα.

156. The modified cell of claim 155, wherein the agent comprises an anti-CD47 antibody.

157. The modified cell of claim 155, wherein the agent comprises an anti-SIRPα antibody.

158. The modified cell of any one of claims 147 to 157, wherein the modified cell comprises an agent capable of preventing or reducing interaction between CD24 and SIGLEC10.

159. The modified cell of claim 158, wherein the agent comprises an anti-CD24 antibody.

160. The modified cell of claim 158, wherein the agent comprises an anti-SIGLEC10 antibody.

161. A pharmaceutical composition comprising the modified cell of any one of claims 147 to 160.

162. A method of treating a cancer, comprising administering the pharmaceutical composition of claim 161.

163. A method of engineering a modified cell, comprising introducing into the cell the polynucleotide of any one of claims 1 to 78, the vector of any one of claims 79 to 84, or the polypeptide of any one of claims 85 to 144,
wherein the cell is a monocyte, a macrophage, or an induced pluripotent stem cell (iPSC).

164. The method of claim 163, wherein the cell is an iPSC.

165. The method of claim 163, wherein the cell is a macrophage.

166. The method of claim 163, wherein the cell is a monocyte.

167. The method of claim 165 or 166, wherein the cell is derived from an iPSC.

168. The method of any one of claims 163 to 167, wherein the cell is a human cell.

169. The method of any one of claims 163 to 168, wherein the cell comprises a genetic disruption of:
(a) a signal regulatory protein alpha (SIRPA) gene;
(b) a cytokine inducible SH2 containing protein (CISH) gene; and/or
(c) a sialic acid-binding Ig-like lectin 10 (SIGLEC10) gene.

170. The method of claim 169, wherein the modified cell comprises genetic disruption of SIRPA.

171. The method of claim 169 or 170, wherein the modified cell comprises genetic disruption of CISH.

172. The method of any one of claims 169 to 171, wherein the modified cell comprises genetic disruption of SIGLEC10.
